(19) United States
(12) Patent Application Publication
(10) Pub. No.: US 2010/0009973 A1
(43) Pub. Date: Jan. 14, 2010

(54) Integrase Inhibitors 3

(75) Inventors: David Ian Rhodes, Heidelberg Heights (AU); Katherine MacFarlane, Heidelberg Heights (AU); Eric Dale Jones, Bentleigh East (AU); William Issa, Nunawading (AU); John Joseph Deadman, Carlton (AU); Neil Choi, Lower Templestowe (AU)

Correspondence Address: DARBY & DARBY P.C., P.O. BOX 770, Church Street Station, New York, NY, 10008-0770, US

(73) Assignee: AVEXA LIMITED, RICHMOND (AU)

(21) Appl. No.: 12/298,502

(22) PCT Filed: Apr. 30, 2007
(86) PCT No.: PCT/AU07/00562

§ 371 Date: Jul. 28, 2009

(30) Foreign Application Priority Data
Apr. 28, 2006 (AU) 2006902229

(51) Int. Cl. C07D 409/04; C07D 417/04; C07D 405/04; C07D 413/14; C07D 417/14; A61K 31/443; A61K 31/4436; A61K 31/444; A61K 31/535; A61K 31/541; A61P 31/12

(52) U.S. Cl. 514/222.2; 546/280.4; 546/270.4; 546/256; 546/283.4; 544/124; 514/336; 514/342; 514/337; 514/333; 546/167; 514/314; 544/3; 514/231.5

(57) ABSTRACT

The present invention provides a method of treatment or prophylaxis of a viral infection in a subject comprising administering to said subject an effective amount of a compound of formula (I) or a pharmaceutically acceptable derivative, salt or prodrug thereof. Compounds of formula (I) are also provided.

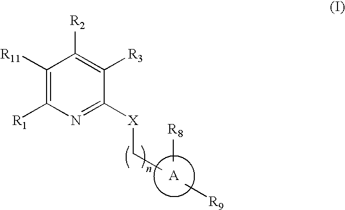

### FIELD OF THE INVENTION

The present invention relates to novel pyridine-based compounds for the treatment of HIV infection.

### BACKGROUND OF THE INVENTION

The retrovirus designated “human immunodeficiency virus” or “HIV” is the etiological agent of a complex disease that progressively destroys the immune system. This disease is known as acquired immune deficiency syndrome or AIDS. As at December 2004, an estimated 40 million people have been infected with HIV world wide and over 3 million deaths are occurring annually.

A feature of retrovirus replication includes the reverse transcription of the viral genome into proviral DNA and its integration into the host cell genome. These steps are required for HIV replication and are mediated by the virus encoded enzymes, reverse transcriptase and integrase respectively.

HIV infection follows a path of the virus particle binding to cell surface receptors and co-receptors resulting in fusion of the virus particle with the cell. The contents of the virus are released into the cytoplasm where reverse transcription of the HIV genome occurs. Through a series of steps a double stranded proviral DNA copy is produced. The proviral DNA is transported to the nucleus in a complex known as the pre integration complex (PIC) which contains integrase and other viral and possibly cellular proteins. Once inside the nucleus the proviral DNA is integrated into the host cell genome via the action of integrase. Once integrated, transcription and translation of the viral genome can occur resulting in the production of viral proteins and a new viral RNA genome. These proteins and genome assemble at the cell surface and, depending on cell type, possibly other intracellular membranous compartments. Assembled particles then bud out from the cell and during, or soon after, this process mature into infectious HIV particles through the action of the viral protease.

The integration of the proviral genome into the host cell genome requires the action of an integrase which carries out this process in at least three steps, possibly four. The first step involves the assembly of the viral genome into a stable nucleoprotein complex, secondly, processing of two nucleotides from the 3′ termini of the genome to give staggered ends with free 3′ OH residues and thirdly the transfer of these ends into the host cell genome. The final step involves the gap filling and repair of the insertion site in the host genome. There is still some conjecture over whether the integrase performs this final step or whether it is carried out by cellular repair enzymes.

Currently HIV infection can be treated with a number of inhibitors on the market which target reverse transcriptase, protease or entry into the cell. Treatment of HIV infection with these, or a combination of these, drugs is known to be an effective treatment for AIDS and similar diseases. Shortcomings with the current inhibitors include the rapid emergence and increase incidence of resistance and numerous side effects and hence there is a need for new classes of inhibitors.

### SUMMARY OF THE INVENTION

In a first aspect, the present invention provides a method of treatment or prophylaxis of a HIV infection in a subject comprising administering to said subject an effective amount of a compound of formula (I) or a pharmaceutically acceptable derivative, salt or prodrug thereof wherein:

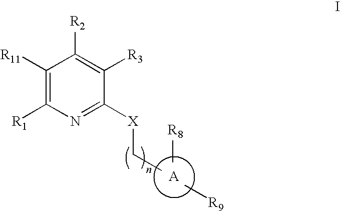

X is selected from —O—, —S—, —S(O)—, —S(O₂)— and NR₄;
R₄ is selected from H and C₁₋₃alkyl;
   n is 0 or 1;
   A is C₆aryl or heteroaryl;
   R₁ is selected from the group consisting of hydrogen, halo, C₆₋₁₀aryl, C₆₋₁₀arylC₁₋₃alkyl, —C₁₋₁₀alkyl-O—C₁₋₁₀alkyl, heterocyclyl, hetereoaryl, C₁₋₁₀alkyl, C₁₋₁₀alkoxy, C₂₋₁₀alkenyl, C₂₋₁₀alkynyl, C₃₋₁₀cycloalkyl, —NR₅R₆, —C₆arylNR₅R₆, —C₆aryl-SO₂—NR₅R₆, —C₆aryl-heterocyclyl, —C₆aryl-SO₂-heterocyclyl; -heteroaryl-R₁₀; -Z-C₁₋₆alkylene-SO₂—R₁₂, -Z-(C₂H₄O)ₚ—R₁₂,
   or R₁ and R₁₁ are joined together to form a C₃₋₄alkylene;
   R₂ is selected from the group consisting of hydrogen, C₆₋₁₀aryl, C₆₋₁₀arylC₁₋₃alkyl, heterocyclyl, hetereoaryl, C₁₋₁₀alkyl, C₂₋₁₀alkenyl, C₂₋₁₀alkynyl, C₃₋₁₀cycloalkyl and —NR₅R₆, -heteroaryl-C₆₋₁₀aryl, -heteroaryl-heteroaryl;
   R₃ is selected from the group consisting of hydrogen, cyano, halo, —NO₂, —C(O)NR₅R₆, —CH₂NR₅R₆, —C(O)R₇ and —CO₂R₇; 4.

Z is absent or is selected from the group consisting of NR₅, O, S, S(O), S(O₂);

p is 1 to 3;

R₅ and R₆ are each independently selected from the group consisting of hydrogen, C₁₋₁₀alkyl, C₃₋₆cycloaklyl, C₆₋₁₀arylC₁₋₃alkyl and C₆₋₁₀aryl;

R₇ is hydrogen or C₁₋₁₀alkyl

R₁₂ is hydrogen or C₁₋₁₀alkyl;

R₈ is zero to two substituents each independently selected from the group consisting of —OH, —SO₂NH₂, —OC(O)R₇, —CO₂R₇, C₁₋₁₀alkyl, C₁₋₁₀alkoxy, halo, —NO₂, and —NR₅R₆;
R₉ is selected from the group consisting of hydrogen, cyano, —SO₂NH₂, —R₁₀, and —C(O)R₁₀;

R₁₀ is selected from OH, —C₁₋₁₀alkyl, —OC₁₋₁₀alkyl, —OC₂₋₁₀alkenyl, and —Y-heteroaryl; and
Y is absent or is selected from —O— and —NR₄—
   R₁₁ is selected form the group consisting of hydrogen, C₁₋₁₀alkyl, C₁₋₁₀alkoxy; or R₁ and R₁₁ are joined together to form a C₃₋₄alkylene.

In a second aspect, there is provided the use of a compound of Formula I or a pharmaceutically acceptable derivative, salt or prodrug thereof in the preparation of a medicament for the treatment or prophylaxis of a HIV infection in a subject.

In a third aspect, the present invention provides a compound of Formula I or a pharmaceutically acceptable derivative, salt or prodrug thereof wherein:

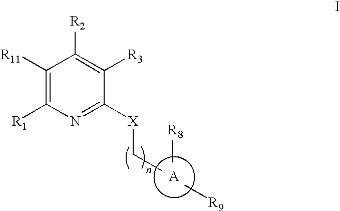

X is selected from —O—, —S—, —S(O)—, —S(O₂)— and NR₄;

R₄ is selected from H and C₁₋₃alkyl;

n is 0 or 1;
A is C₆aryl or heteroaryl;
R₁ is selected from the group consisting of hydrogen, halo, C₆₋₁₀aryl, C₆₋₁₀arylC₁₋₃alkyl, —C₁₋₁₀alkyl-O—C₁₋₁₀alkyl, heterocyclyl, hetereoaryl, C₁₋₁₀alkyl, C₁₋₁₀alkoxy, C₂₋₁₀alkenyl, C₂₋₁₀alkynyl, C₃₋₁₀cycloalkyl, —NR₅R₆, —C₆arylNR₅R₆, —C₆aryl-SO₂—NR₅R₆, —C₆aryl-heterocyclyl, —C₆aryl-SO₂-heterocyclyl; -heteroaryl-R₁₀; -Z-C₁₋₆alkylene-SO₂—R₁₂, -Z-(C₂H₄₀)ₚ—R₁₂,
or R₁ and R₁₁ are joined together to form a C₃₋₄alkylene;
R₂ is selected from the group consisting of hydrogen, C₆₋₁₀aryl, C₆₋₁₀arylC₁₋₃alkyl, heterocyclyl, hetereoaryl, C₁₋₁₀alkyl, C₂₋₁₀alkenyl, C₂₋₁₀alkynyl, C₃₋₁₀cycloalkyl and —NR₅R₆, -heteroaryl-C₆₋₁₀aryl, -heteroaryl-heteroaryl;
R₃ is selected from the group consisting of hydrogen, cyano, halo, —NO₂, —C(O)NR₅R₆, —CH₂NR₅R₆, —C(O)R₇ and —CO₂R₇;

Z is absent or is selected from the group consisting of NR₅, O, S, S(O), S(O₂);

p is 1 to 3;

R₅ and R₆ are each independently selected from the group consisting of hydrogen, C₁₋₁₀alkyl, C₃₋₆cycloaklyl, C₆₋₁₀arylC₁₋₃alkyl and C₆₋₁₀aryl;

R₇ is hydrogen or C₁₋₁₀alkyl

R₁₂ is hydrogen or C₁₋₁₀alkyl;

R₈ is zero to two substituents each independently selected from the group consisting of —OH, —SO₂NH₂, —OC(O)R₇, —CO₂R₇, C₁₋₁₀alkyl, C₁₋₁₀alkoxy, halo, —NO₂, and —NR₅R₆;
R₉ is selected from the group consisting of hydrogen, cyano, —SO₂NH₂, —R₁₀, and —C(O)R₁₀;

R₁₀ is selected from OH, —C₁₋₁₀alkyl, —OC₁₋₁₀alkyl, —OC₂₋₁₀alkenyl, and —Y-heteroaryl; and
Y is absent or is selected from —O— and —NR₄—
   R₁₁ is selected form the group consisting of hydrogen, C₁₋₁₀alkyl, C₁₋₁₀alkoxy; or R₁ and R₁₁ are joined together to form a C₃₋₄alkylene.

In a fourth aspect, the present invention provides a pharmaceutical composition comprising a compound according to the third aspect and a pharmaceutically acceptable carrier, diluent or excipient.

### DETAILED DESCRIPTION OF THE INVENTION

The compounds of the present invention display anti-viral activity. The present inventors have found that the compounds inhibit HIV replication in infected cells and have also shown that the compounds inhibit the activity of HIV integrase in vitro.

Accordingly, in a first aspect, the present invention provides a method of treatment or prophylaxis of a viral infection in a subject comprising administering to said subject an effective amount of a compound of formula I or a pharmaceutically acceptable derivative, salt or prodrug thereof wherein:

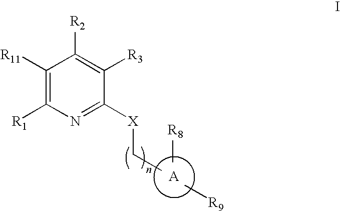

X is selected from —O—, —S—, —S(O)—, —S(O₂)— and NR₄;

R₄ is selected from H and C₁₋₃alkyl;

n is 0 or 1;
A is C₆aryl or heteroaryl;
R₁ is selected from the group consisting of hydrogen, halo, C₆₋₁₀aryl, C₆₋₁₀arylC₁₋₃alkyl, —C₁₋₁₀alkyl-O—C₁₋₁₀alkyl, heterocyclyl, hetereoaryl, C₁₋₁₀alkyl, C₁₋₁₀alkoxy, C₂₋₁₀alkenyl, C₂₋₁₀alkynyl, C₃₋₁₀cycloalkyl, —NR₅R₆, —C₆arylNR₅R₆, —C₆aryl-SO₂—NR₅R₆, —C₆aryl-heterocyclyl, —C₆aryl-SO₂-heterocyclyl; -heteroaryl-R₁₀; -Z-C₁₋₆alkylene-SO₂—R₁₂, -Z-(C₂H₄O)ₚ—R₁₂,
or R₁ and R₁₁ are joined together to form a C₃₋₄alkylene;
R₂ is selected from the group consisting of hydrogen, C₆₋₁₀aryl, C₆₋₁₀arylC₁₋₃alkyl, heterocyclyl, hetereoaryl, C₁₋₁₀alkyl, C₂₋₁₀alkenyl, C₂₋₁₀alkynyl, C₃₋₁₀cycloalkyl and —NR₅R₆, -heteroaryl-C₆₋₁₀aryl, -heteroaryl-heteroaryl;
R₃ is selected from the group consisting of hydrogen, cyano, halo, —NO₂, —C(O)NR₅R₆, —CH₂NR₅R₆, —C(O)R₇ and —CO₂R₇;

Z is absent or is selected from the group consisting of NR₅, O, S, S(O), S(O₂);

p is 1 to 3;

R₅ and R₆ are each independently selected from the group consisting of hydrogen, C₁₋₁₀alkyl, C₃₋₆cycloaklyl, C₆₋₁₀arylC₁₋₃alkyl and C₆₋₁₀aryl;

R₇ is hydrogen or C₁₋₁₀alkyl

R₁₂ is hydrogen or C₁₋₁₀alkyl;

R₈ is zero to two substituents each independently selected from the group consisting of —OH, —SO₂NH₂, —OC(O)R₇, —CO₂R₇, C₁₋₁₀alkyl, C₁₋₁₀alkoxy, halo, —NO₂, and —NR₅R₆;
R₉ is selected from the group consisting of hydrogen, cyano, —SO₂NH₂, —R₁₀, and —C(O)R₁₀;

R₁₀ is selected from OH, —C₁₋₁₀alkyl, —OC₁₋₁₀alkyl, —OC₂₋₁₀alkenyl, and —Y-heteroaryl; and
Y is absent or is selected from —O— and —NR₄—
   R₁₁ is selected form the group consisting of hydrogen, C₁₋₁₀alkyl, C₁₋₁₀alkoxy; or R₁ and R₁₁ are joined together to form a C₃₋₄alkylene.

Preferably, R₁ is selected from the group consisting of C₆₋₁₀aryl and heteroaryl.

Preferably, R₂ is selected from the group consisting of C₆₋₁₀aryl and heteroaryl.

Preferably, n is 1.

Preferably, R₁₁ is hydrogen.

Preferably, A is phenyl. More preferably, A is 1,4-substituted phenyl.

In another preferred from, A is pyrdinyl, preferably 1,4-substituted pyridinyl.

In a yet further preferred form, A is heteroaryl selected from the group consisting of pyrrolidinyl, furanyl, and thiophene. Preferably, the heteroaryl is 2,5-substituted. Examples of compounds of this type which would be contemplated as within the scope of the present invention include:

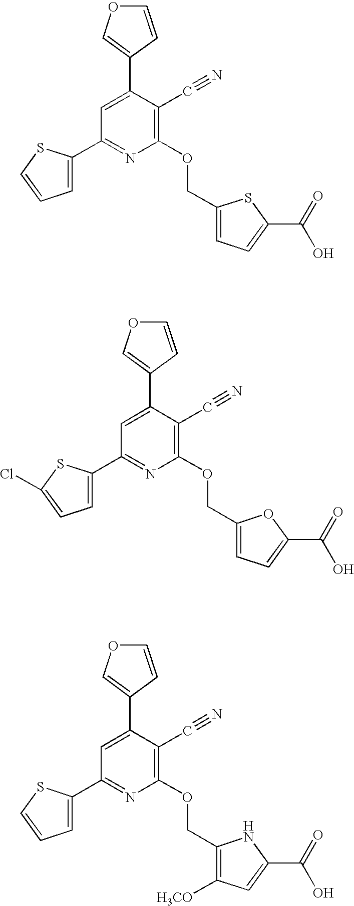

Examples of compounds in which R₁ and R₁₁ are joined together to form a C₃₋₄alkylene would include:

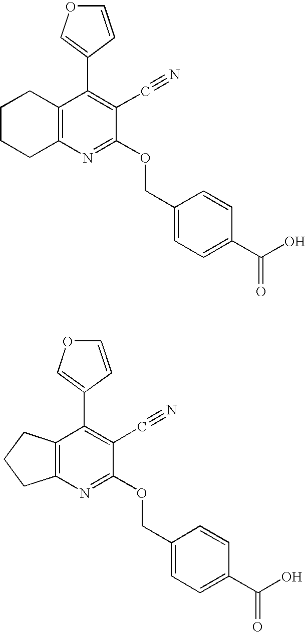

Examples of compounds where R₁ is —C₆aryl-SO₂-heterocyclyl would include:

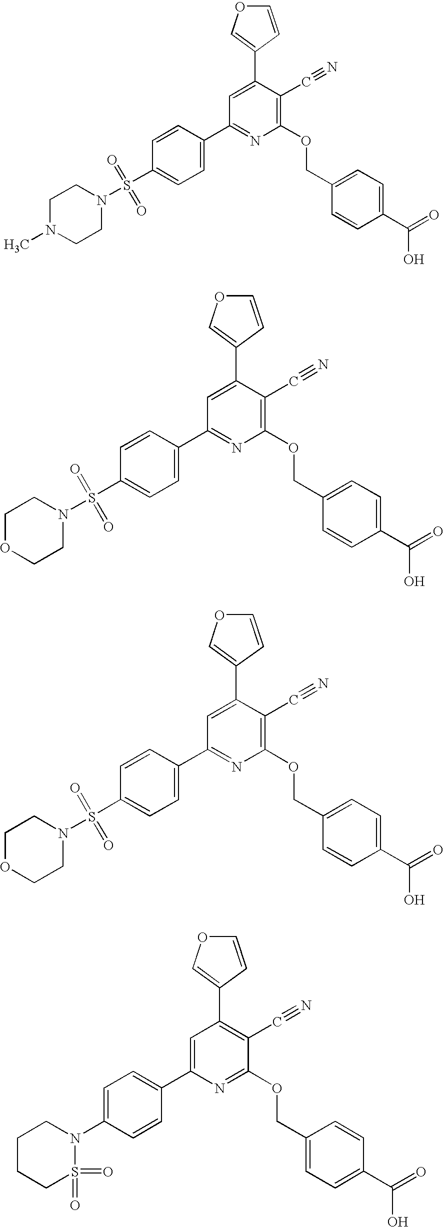

Preferably, each C₁₋₁₀alkyl group is C₁₋₆alkyl, more preferably C₁₋₃alkyl.

Preferably, each C₂₋₁₀alkenyl group is preferably C₂₋₆alkenyl, more preferably C₂₋₃alkenyl and even more preferably allyl.

Preferably, the compound of formula I is:

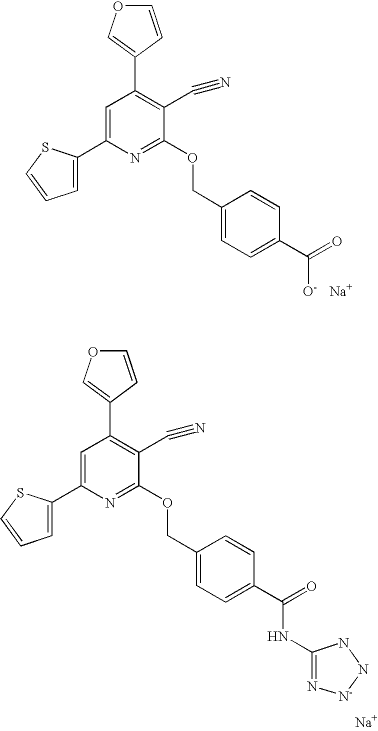

In a second aspect, there is provided the use of a compound of Formula I or a pharmaceutically acceptable derivative, salt or prodrug thereof in the preparation of a medicament for the treatment or prophylaxis of a HIV infection in a subject.

In a third aspect, the present invention provides a compound of Formula I or a pharmaceutically acceptable derivative, salt or prodrug thereof wherein:

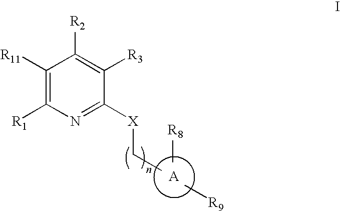

X is selected from —O—, —S—, —S(O)—, —S(O₂)— and NR₄;
R₄ is selected from H and C₁₋₃alkyl;
   n is 0 or 1;
   A is C₆aryl or heteroaryl;
   R₁ is selected from the group consisting of hydrogen, halo, C₆₋₁₀aryl, C₆₋₁₀arylC₁₋₃alkyl, —C₁₋₁₀alkyl-O—C₁₋₁₀alkyl, heterocyclyl, hetereoaryl, C₁₋₁₀alkyl, C₁₋₁₀alkoxy, C₂₋₁₀alkenyl, C₂₋₁₀alkynyl, C₃₋₁₀cycloalkyl, —NR₅R₆, —C₆arylNR₅R₆, —C₆aryl-SO₂—NR₅R₆, —C₆aryl-heterocyclyl, —C₆aryl-SO₂-heterocyclyl; -heteroaryl-R₁₀; -Z-C₁₋₆alkylene-SO₂—R₁₂, -Z-(C₂H₄₀)ₚ—R₁₂,
   or R₁ and R₁₁ are joined together to form a C₃₋₄alkylene;
   R₂ is selected from the group consisting of hydrogen, C₆₋₁₀aryl, C₆₋₁₀arylC₁₋₃alkyl, heterocyclyl, hetereoaryl, C₁₋₁₀alkyl, C₂₋₁₀alkenyl, C₂₋₁₀alkynyl, C₃₋₁₀cycloalkyl and —NR₅R₆, -heteroaryl-C₆₋₁₀aryl, -heteroaryl-heteroaryl;

R₃ is selected from the group consisting of hydrogen, cyano, halo, —NO₂, —C(O)NR₅R₆, —CH₂NR₅R₆, —C(O)R₇ and —CO₂R₇;

Z is absent or is selected from the group consisting of NR₅, O, S, S(O), S(O₂);

p is 1 to 3;

R₅ and R₆ are each independently selected from the group consisting of hydrogen, C₁₋₁₀alkyl, C₃₋₆cycloaklyl, C₆₋₁₀arylC₁₋₃alkyl and C₆₋₁₀aryl;

R₇ is hydrogen or C₁₋₁₀alkyl

R₁₂ is hydrogen or C₁₋₁₀alkyl;

R₈ is zero to two substituents each independently selected from the group consisting of —OH, —SO₂NH₂, —OC(O)R₇, —CO₂R₇, C₁₋₁₀alkyl, C₁₋₁₀alkoxy, halo, —NO₂, and —NR₅R₆;
R₉ is selected from the group consisting of hydrogen, cyano, —SO₂NH₂, —R₁₀, and —C(O)R₁₀;

R₁₀ is selected from OH, —C1-10alkyl, —OC₁₋₁₀alkyl, —OC₂₋₁₀alkenyl, and —Y-heteroaryl; and
Y is absent or is selected from —O— and —NR₄—
   R₁₁ is selected form the group consisting of hydrogen, C₁₋₁₀alkyl, C₁₋₁₀alkoxy; or R₁ and R₁₁ are joined together to form a C₃₋₄alkylene.

Preferably, R₁ is selected from the group consisting of C₆₋₁₀aryl and heteroaryl.

Preferably, R₂ is selected from the group consisting of C₆₋₁₀aryl and heteroaryl.

Preferably, n is 1.

Preferably, R₁₁ is hydrogen.

Preferably, A is phenyl. More preferably, A is 1,4-substituted phenyl.

In another preferred from, A is pyrdinyl, preferably 1,4-substituted pyridinyl.

In a yet further preferred form, A is heteroaryl selected from the group consisting of pyrrolidinyl, furanyl, and thiophene. Preferably, the heteroaryl is 2,5-substituted. Examples of compounds of this type which would be contemplated as within the scope of the present invention include:

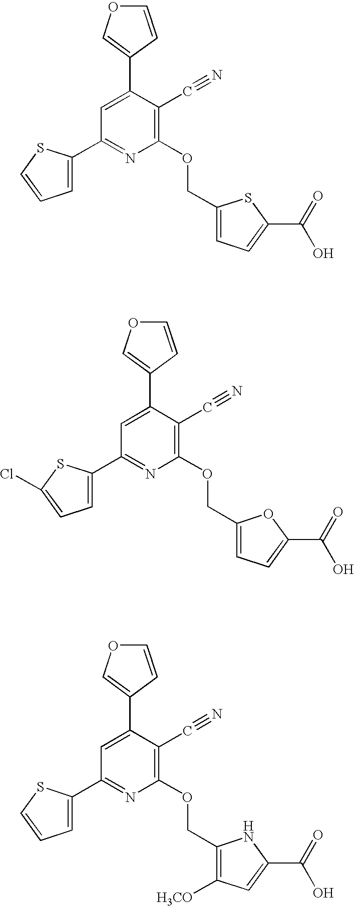

Examples of compounds in which R₁ and R₁₁ are joined together to form a C₃₋₄alkylene would include:

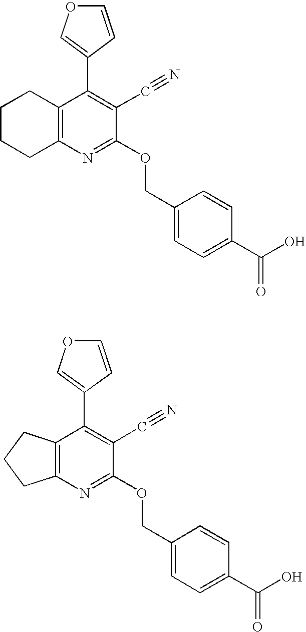

Examples of compounds where R₁ is —C₆aryl-SO₂-heterocyclyl would include:

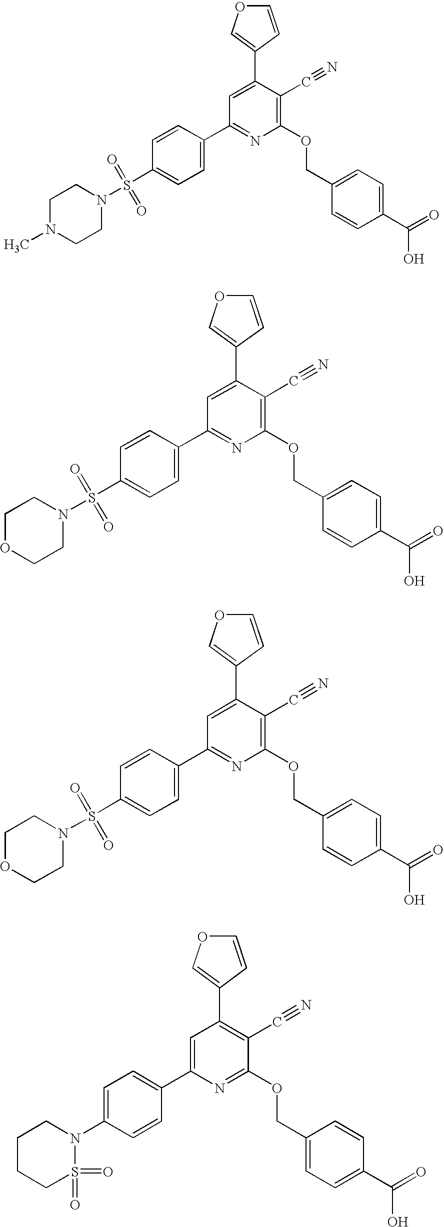

Preferably, each C₁₋₁₀alkyl group is C₁₋₆alkyl, more preferably C₁₋₃alkyl.

Preferably, each C₂₋₁₀alkenyl group is preferably C₂₋₆alkenyl, more preferably C₂₋₃alkenyl and even more preferably allyl.

Preferably, the compound of formula I is:

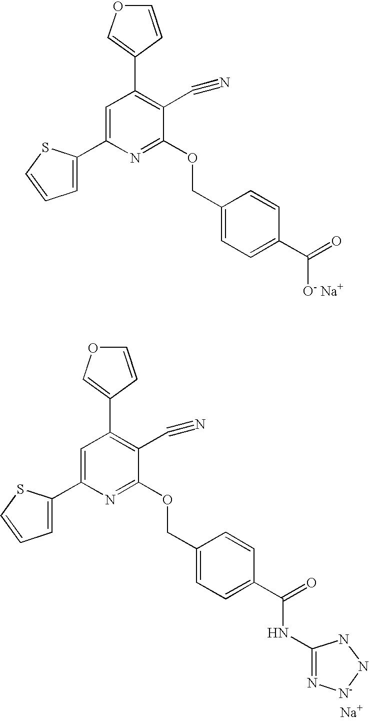

In a fourth aspect, the present invention provides pharmaceutical composition comprising a compound according to the third aspect and a pharmaceutically acceptable carrier, diluent or excipient.

As used herein, the term “halo” or “halogen” refers to fluorine (fluoro), chlorine (chloro), bromine (bromo) or iodine (iodo).

As used herein, the term “alkyl” either used alone or in compound terms such as NH(alkyl) or N(alkyl)₂, refers to monovalent straight chain or branched hydrocarbon groups. For example, suitable alkyl groups include, but are not limited to methyl, ethyl, propyl, isopropyl, n-butyl, sec-butyl, tert-butyl, pentyl, 2-methylbutyl, 3-methylbutyl, n-hexyl, 2-, 3- or 4-methylpentyl, 2-ethylbutyl, n-hexyl or 2-, 3-, 4- or 5-methylpentyl.

As used herein, the term “alkenyl” refers to straight chain or branched hydrocarbon groups having one or more double bonds between carbon atoms. Suitable alkenyl groups include, but are not limited to ethenyl, propenyl, isopropenyl, butenyl, pentenyl and hexenyl.

The term “alkynyl” as used herein, refers to straight chain or branched hydrocarbon groups containing one or more triple bonds. Suitable alkynyl groups include, but are not limited to ethynyl, propynyl, butynyl, pentynyl and hexenyl.

The term “cycloalkyl” as used herein, refers to cyclic hydrocarbon groups. Suitable cycloalkyl groups include, but are not limited to cyclopropyl, cyclobutyl, cyclopentyl and cyclohexyl.

The term “aryl” as used herein, refers to a C₆-C₁₀ aromatic hydrocarbon group, for example phenyl or naphthyl.

The term “arylalkyl” includes, for example, benzyl.

The term “heterocycle” when used alone or in compound words includes monocyclic, polycyclic, fused or conjugated hydrocarbon residues, preferably C₃₋₆, wherein one or more carbon atoms (and where appropriate, hydrogen atoms attached thereto) are replaced by a heteroatom so as to provide a non-aromatic residue. Suitable heteroatoms include O, N and S, S(O) and S(O₂). Where two or more carbon atoms are replaced, this may be by two or more of the same heteroatom or by different heteroatoms. Suitable examples of heterocyclic groups may include pyrrolidinyl, piperidyl, piperazinyl, morpholino, quinolinyl, isoquinolinyl, thiomorpholino, dioxanyl, tetrahydrofuranyl, tetrahydropyranyl, tetrahydropyrrolyl, lactams, sultams etc.

Preferred sultams include:

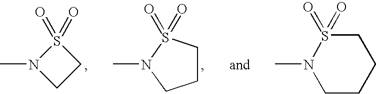

The term “heteroaryl” includes a 5- or 6-membered heteroaromatic ring containing one or more heteroatoms selected from O, N and S. Suitable examples of heteroaryl groups include tetrazolyl, 1,2,3-triazolyl, 1,2,4-triazolyl, imidazolyl, pyrazolyl, pyridinyl, pyrimidinyl, oxazolyl, oxadiazolyl etc. The heteroaromatic ring may be fused to another 5- or 6-membered aromatic ring to form a bicyclic aromatic system eg benzofuran.

Each alkyl, alkenyl, alkynyl, cycloalkyl, aryl, heterocyclyl, or heteroaryl group may be optionally substituted with C₁-C₃alkyl, C₆aryl, alkylaryl, OH, OC₁-C₃alkyl, halo, CN, NO₂, CO₂H, CO₂C₁-C₃alkyl, CONH₂, CONH(C₁-C₃alkyl), CON(C₁-C₃alkyl)₂, trifluoromethyl, NH₂, NH(C₁-C₃alkyl) or N(C₁-C₃alkyl)₂. For example, an optionally substituted aryl group may be 4-methylphenyl or 4-hydroxyphenyl group, and an optionally substituted alkyl group may be 2-hydroxyethyl, trifluoromethyl, or difluoromethyl. Each aryl may optionally be fused with a dioxolane ring. Any of the above substituents may additionally be substituted by optional substituents.

Optional substituents also includes suitable nitrogen protecting groups (see “Protective Groups in Organic Synthesis” Theodora Greene and Peter Wuts, third edition, Wiley hiterscience, 1999).

The salts of the compound of formula I are preferably pharmaceutically acceptable, but it will be appreciated that non-pharmaceutically acceptable salts also fall within the scope of the present invention, since these are useful as intermediates in the preparation of pharmaceutically acceptable salts.

The term “pharmaceutically acceptable derivative” may include any pharmaceutically acceptable salt, hydrate or prodrug, or any other compound which upon administration to a subject, is capable of providing (directly or indirectly) a compound of formula I or an antibacterially active metabolite or residue thereof.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

This invention also encompasses prodrugs of compounds of formula I. This invention also encompasses methods of treating or preventing disorders in a subject that can be treated or prevented by the inhibition of AIDS and other disorders that can be treated by inhibition of the integrase enzyme by administering prodrugs of compounds of the formula (I). Compounds of formula I having free amino, amido, hydroxy or carboxylic groups can be converted into prodrugs.

Prodrugs include compounds wherein an amino acid residue, or a polypeptide chain of two or more (eg, two, three or four) amino acid residues which are covalently joined through peptide bonds to free amino, hydroxy and carboxylic acid groups of compounds of formula I. The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of formula I through the carbonyl carbon prodrug sidechain. Prodrugs also include phosphate derivatives of compounds of formula I (such as acids, salts of acids, or esters) joined through a phosphorus-oxygen bond to a free hydroxyl of compounds of formula I.

It will also be recognised that the compounds of formula I may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

In a fourth aspect, the present invention provides a pharmaceutical composition comprising a compound according to the third aspect and a pharmaceutically acceptable carrier, diluent or excipient.

The compositions of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compounds of the present invention may be administered by any suitable means, for example, parenterally, such as by subcutaneous, intravenous, intramuscular, or intracisternal injection or infusion techniques (e.g., as sterile injectable aqueous or non-aqueous solutions or suspensions).

Pharmaceutical formulations include those for oral, rectal, nasal, topical (including buccal and sub-lingual), vaginal or parenteral (including intramuscular, sub-cutaneous and intravenous) administration or in a form suitable for administration by inhalation or insufflation. The compounds of the invention, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, in the form of suppositories for rectal administration; or in the form of sterile injectable solutions for parenteral (including subcutaneous) use.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The subjects treated in the above method are mammals, including, but not limited to, cows, sheep, goats, horses, dogs, cats, guinea pigs, rats or other bovine, ovine, equine, canine, feline, rodent or murine species, and preferably a human being, male or female.

The term “effective amount” means the amount of the subject composition that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical composition and method of the present invention may further comprise other therapeutically active compounds which are usually applied in the treatment of the above mentioned pathological conditions. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

When other therapeutic agents are employed in combination with the compounds of the present invention they may be used for example in amounts as noted in the Physician Desk Reference (PDR) or as otherwise determined by one of ordinary skill in the art.

In the treatment or prevention of conditions which require HIV inhibition or HIV integrase enzyme inhibition an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In order that the nature of the present invention may be more clearly understood preferred forms thereof will now be described by reference to the following non-limiting Examples.

### EXAMPLES

### Methods

### HPLC Conditions

All HPLC measurements were performed on a Waters 2690 Alliance System.

### Method 1:

### Column:

Waters C18 5 uM Symmetry Column (Part # WAT046980) at 30° C., flow rate 0.7 mL/min, spectra measured at 254 nM

### Buffers:

Buffer A: 100% water, Buffer B: 100% acetonitrile, Buffer C: 2% aqueous formic acid
Gradient: (linear gradient curve 6)

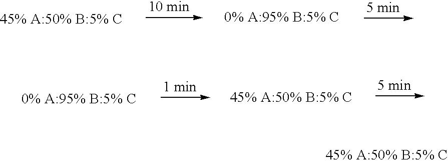

### Method 2:

### Column:

Merck C18 Chromolith Column (Part # 1.02129.0001) at 30° C., flow rate 4 mL/min, spectra measured at 254 nM

### Buffers:

Buffer A: 100% water, Buffer B: 100% acetonitrile, Buffer C: 2% aqueous TFA
Gradient: (linear gradient curve 6)

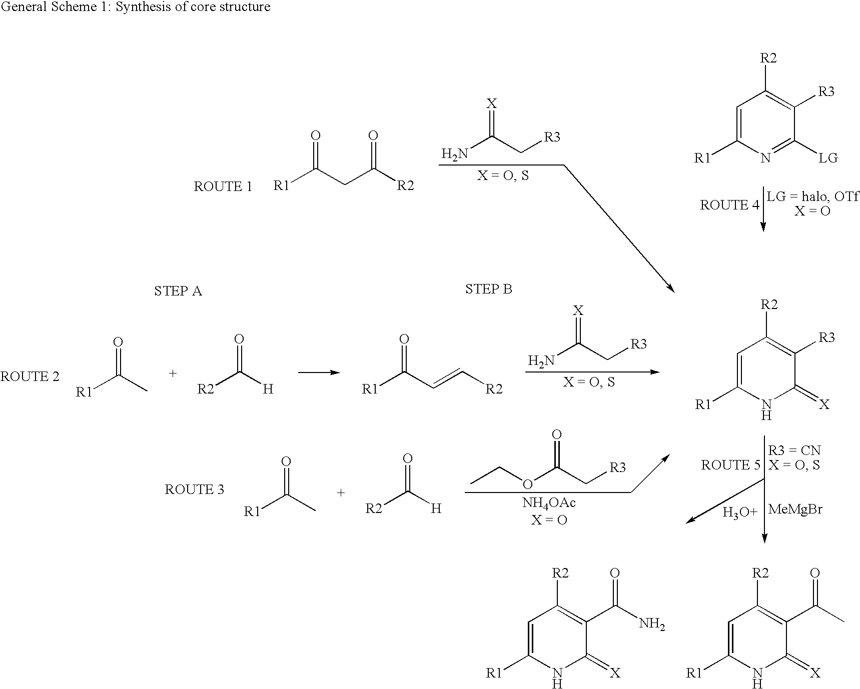

### Example 1

### Preparation of 4,6-Diphenyl-2-thioxo-1,2-dihydro-3-pyridinecarbonitrile (Route 1)

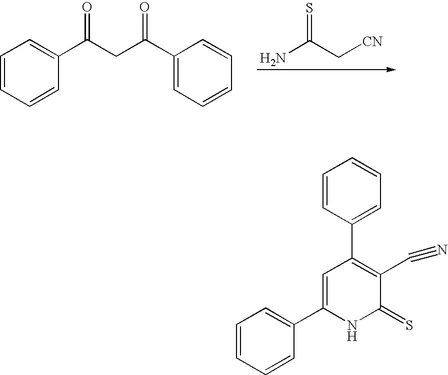

A suspension of cyanothioamide (1.0 g, 9.98 mmol) and dibenzoyl methane (2.24 mg, 9.98 mmol) in dry ethanol (20 mL) was treated with triethylamine (catalytic, 500 μL) then refluxed for 2 hours. Reaction mixture was allowed to cool to room temperature to give a yellow precipitate which was filtered to afford 4,6-diphenyl-2-thioxo-1,2-dihydro-3-pyridinecarbonitrile as a yellow solid (1.02 g, 35%).

¹H NMR (300 MHz, D⁶DMSO) δ 7.11 (1H, s, pyridylH), 7.56 (6H, m, ArH), 7.73 (2H, m, ArH), 7.86 (2H, d, J=7.2 Hz, ArH);

MS (ESI⁺) m/z 289 (M+1); MS (ESI⁻) m/z 287 (M−1).

### Example 2

### Preparation of 4-Furan-2-yl-6-thiophen-2-yl-1H-pyridin-2-one and 4-furan-2-yl-6-thiophen-2-yl-2-thioxo-1,2-dihydro-pyridine-3-carbonitrile (Route 2)

### 2.1 Preparation of (E)-3-Furan-2-yl-1-thiophen-2-yl-propenone

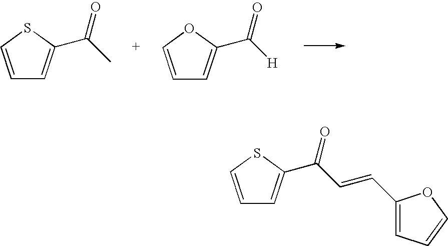

Aqueous sodium hydroxide (2.0 M, 30 mL) was added dropwise to a solution of 2-acetyl thiophene (10 g, 8.65 mL, 79.3 mmol) and 2-furan-carboxaldehyde (6.92 g, 72.0 mmol) in ethanol (50 mL). After stirring overnight at room temperature the mixture was diluted by addition of (500 mL) and extracted with ethyl acetate (250 mL). The organic phase was dried (Na₂SO₄), filtered and allowed to stand overnight at 0° C. The resulting crystals were filtered and washed with hexane (25 mL) and ethanol (10 mL) to afford (E)-3-furan-2-yl-1-thiophen-2-yl-propenone (10.3 g, 70%).

MS (ESI⁺) m/z 205 (M+1)

### 2.2: Preparation of 4-Furan-2-yl-6-thiophen-2-yl-1H-pyridin-2-one

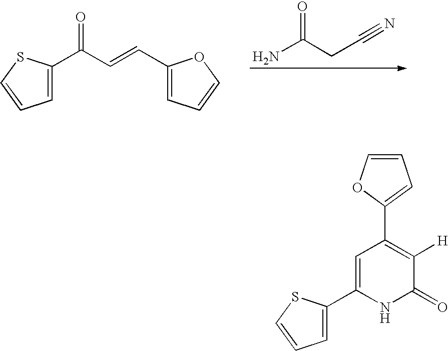

All fumes from this reaction were vented through a bleach trap:

A steady stream of nitrogen was bubbled through a solution of (E)-3-furan-2-yl-1-thiophen-2-yl-propenone (1.0 g, 4.90 mmol) and 2-cyano-thioacetamide (453 mg, 5.39 mmol) in DMSO (14 mL). The mixture was cooled to 0° C. before portion wise addition of potassium tert-butoxide (1.65 g, 14.7 mmol) over 20 minutes. The reaction was warmed to 90° C. and stirred vigorously for 3 hr, still bubbling N₂ through. The reaction was cooled to room temperature and slowly transferred into 4 M aqueous hydrochloric acid (65 mL) cooled in an ice bath (N.B. liberation of HCN)— keeping the temperature below 20° C. This solution was stirred until the evolution of gas ceased (approx. 10 min) and filtered, washing the precipitate with water and ethanol to give pure product (983 mg, 83% yield), as a pale brown solid.

¹H NMR (300 MHz, CDCl₃) δ 7.87, m, 1H, Ar—H, 7.84, m, 1H, Ar—H, 7.67, dd, 1H, J 1.2, 5.1 Hz, Ar—H, 7.31, m, 2H, Ar—H, 7.17, dd, 1H, J 3.9, 5.1 Hz, Ar—H, 6.68, m, 2H, Ar—H.

MS (ESI⁺) m/z 244 (M+1)

### 2.3: Preparation of 4-Furan-2-yl-6-thiophen-2-yl-2-thioxo-1,2-dihydro-pyridine-3-carbonitrile

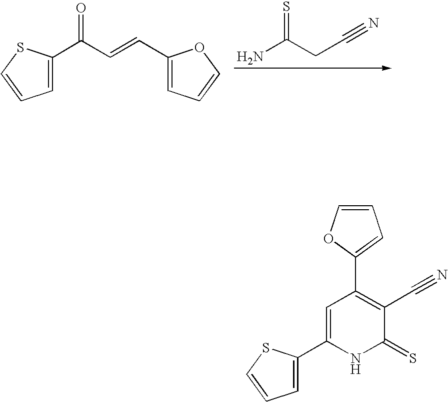

All fumes from this reaction were vented through a bleach trap:

A steady stream of oxygen was bubbled through a solution of (E)-3-furan-2-yl-1-thiophen-2-yl-propenone (1.0 g, 4.90 mmol) and 2-cyano-thioacetamide (540 mg, 5.39 μmmol) in DMSO (14 mL). The mixture was cooled to 0° C. before portionwise addition of potassium tert-butoxide (1.65 g, 14.7 mmol) over 15 minutes. The reaction was warmed to 50° C. and stirred vigorously, still bubbling O₂ through. On completion, the reaction was cooled to room temperature and slowly transferred into 4M HCl (65 mL) cooled in an ice bath (N.B. liberation of HCN)—keeping the temperature below 20° C. This solution was stirred until the evolution of gas ceased (approx. ½ hr) and filtered, washing the precipitate with water and ethanol. The precipitate was triturated with ether and filtered. The precipitate was triturated with hot glacial acetic acid and filtered on cooling to give pure product (617 mg, 44% yield), as a mixture of monomer and dimer, as an orange solid.

¹H NMR (CDCl₃) δ 7.70, s, 1H, Ar—H, 7.58, m, 1H, Ar—H, 7.52, dd, 2H, J 1.2, 3.9 Hz, Ar—H, 7.24, dd, 1H, J 1.2, 5.1 Hz, Ar—H, 6.91, dd, 1H, J 3.6, 5.1 Hz, Ar—H, 6.55, dd, 1H, J 1.8, 3.6 Hz, Ar—H.

MS (ESI⁺) m/z 567 (dimer M+1)

### Example 3

### Preparation of 4-Furan-3-yl-2-oxo-6-thiophen-2-yl-1,2-dihydro-pyridine-3-carbonitrile (Route 3)

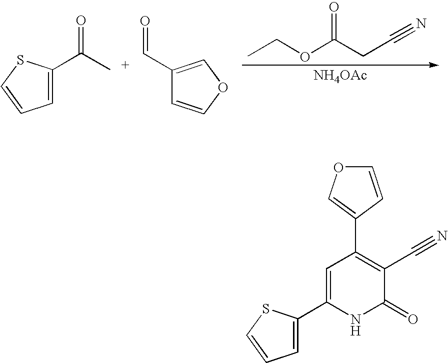

3-Furaldehyde (4.8 g, 50.0 mmol), 2-acetylthiophene (8.26 g, 65.5 mmol), ethyl cyanoacetate (5.66 g, 50.0 mmol) and ammonium acetate (37.19 g, 482.5 mmol) were placed into flask and dissolved in absolute ethanol (50 mL). The reaction mixture was stirred at room temperature for 3 d where a yellow solid formed. The reaction was filtered and the yellow solid was washed with water then with ethanol and then suction dried for 1 h to give the product as a fluffy yellow solid, 6.1 g (46%).

¹H NMR (D⁶DMSO, 300 MHz) δ 12.67 (bs, 1H, hetero-H), 8.58 (s, 1H, H-2 of furan), 8.05 (dd, J=3.9 Hz, 1.2 Hz, 1H, H-5 of thiophene), 7.93 (t, J=1.5 Hz, 1H, H-4 of furan), 7.88 (app d, J=4.5 Hz, 1H, H-5 of furan), 7.26 (dd, J=4.8 Hz, 3.6 Hz, 1H, H-4 of thiophene), 7.22 (dd, J=1.8 Hz, 0.9 Hz, 1H, H-3 of thiophene).

HPLC_{method2} 98.92%/2.18 min.

MS (ESI⁺) m/z 291 (M+23).

### Example 4

### Preparation of trifluoro-methanesulfonic acid 3-cyano-4-furan-3-yl-6-thiophen-2-yl-pyridin-2-yl ester and 2-Bromo-4-furan-2-yl-6-thiophen-2-yl-nicotinonitrile (Route 4)

### 4.1: Preparation of Trifluoro-methanesulfonic acid 3-cyano-4-furan-3-yl-6-thiophen-2-yl-pyridin-2-yl ester

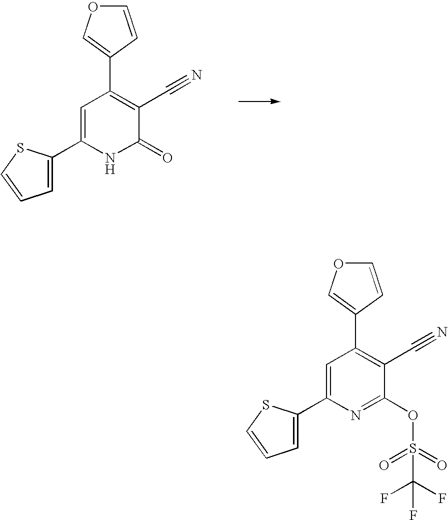

4-Furan-3-yl-2-oxo-6-thiophen-2-yl-1,2-dihydro-pyridine-3-carbonitrile (2.00 g, 7.45 mol) was placed into reaction flask along with dry pyridine (20 mL), stirred at 0° C. under N₂. Triflic anhydride (2.5 mL, 14.9 mmol) was added to the suspension reaction dropwise. Some fuming on addition of triflic anhydride. The initial bright yellow colour darkened after a few minutes stirring, the suspension slowly converted to a dark brown solution over a period of 15 min at 0° C. After which the bath was removed and the reaction mixture was stirred whilst warmed to room temperature and stirred at this temperature for 0.5 h, then evaporated under reduced pressure to give a black/brown solid which was taken up in chloroform. The chloroform solution was loaded onto a silica column and eluted with chloroform. Fractions containing product were collected and evaporated under reduced pressure to give an off-white solid, 1.753 g, 58.7%.

¹H NMR (CDCl₃, 300 MHz) δ 8.33 (s, 1H, H-2 of furan), 7.83 (d, J=3.6 Hz, 1H, H-5 of thiophene), 7.69 (s, 1H, pyridyl H). 7.63 (m, 2H, H-4 and H-5 of furan), 7.21 (app t, 1H, H-4 of thiophene), 6.97 (s, 1H, H-2 of thiophene).

HPLC_{method2} 97.5%/2.86 min.

MS (ESI⁺) m/z 401 (M+1).

### 4.2: Preparation of 2-Bromo-4-furan-2-yl-6-thiophen-2-yl-nicotinonitrile

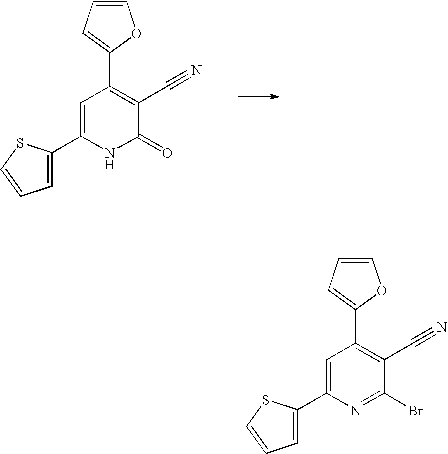

4-Furan-2-yl-2-oxo-6-thiophen-2-yl-1,2-dihydro-pyridine-3-carbonitrile (2.51 g, 9.34 mmol), phosphorous pentoxide (3.18 g, 22.4 mmol), tetrabutylammonium bromide (3.39 g, 10.8 mmol) in dry toluene (120 mL) were heated to reflux for 18 h. The mixture as allowed to cool to room temperature and concentrated n vacuo. Residue was subjected to flash chromatography (chloroform) to afford 2-bromo-4-furan-2-yl-6-thiophen-2-yl-nicotinonitrile (1.2 g, 39%) as a yellow solid.

¹H (300 MHz, CDCl₃) δ 6.66 (dd, J=3.7, 1.8 Hz, 1H, H4-furan), 7.17 (dd, J=5.0, 3.7 Hz, 1H, H4-thiophene), 7.56 (dd, J=5.0, 0.9 Hz, 1H, H3-thiophene), 7.68 (7.70 (dd, J=3.7, 0.9 Hz, 1H, H5-thiophene or H5-furan), 7.81 (dd, J=3.7, 0.9 Hz, 1H, H5-thiophene or H5-furan), 8.01 (s, 1H, H5-pyridine).

HPLC_{method 2} 99.1%/2.27 min

MS (ESP) m/z 333 (M[Br⁸¹]+1), 331 (M[Br⁷⁹]+1)

### Example 5

### Preparation of 3-Acetyl-4-furan-2-yl-6-thiophen-2-yl-1H-pyridin-2-one 4-Isopropyl-2-oxo-6-thiophen-2-yl-1,2-dihydro-pyridine-3-carboxylic acid amide (Route 5)

### 5.1: Preparation of 3-Acetyl-4-furan-2-yl-6-thiophen-2-yl-1H-pyridin-2-one

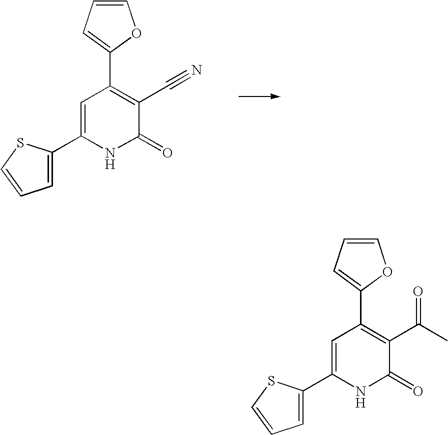

4-Furan-2-yl-2-oxo-6-thiophen-2-yl-1,2-dihydro-pyridine-3-carbonitrile was treated with methylmagnesium bromide to afford 3-acetyl-4-furan-2-yl-6-thiophen-2-yl-1H-pyridin-2-one

### 5.2: Preparation of 4-Isopropyl-2-oxo-6-thiophen-2-yl-1,2-dihydro-pyridine-3-carboxylic acid amide

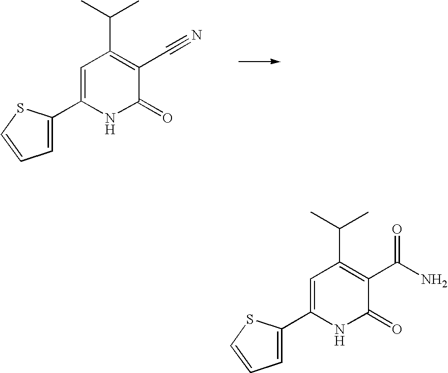

4-Isopropyl-2-oxo-6-thiophen-2-yl-1,2-dihydro-pyridine-3-carbonitrile was treated with 85% aqueous sulphuric acid at 100° C. for 20 min to afford 4-isopropyl-2-oxo-6-thiophen-2-yl-1,2-dihydro-pyridine-3-carboxylic acid amide.

### Synthesis of Substituted Acetophenones

### Example 6

### Preparation of 1-[4-(Morpholine-4-sulfonyl)-phenyl]-ethanone

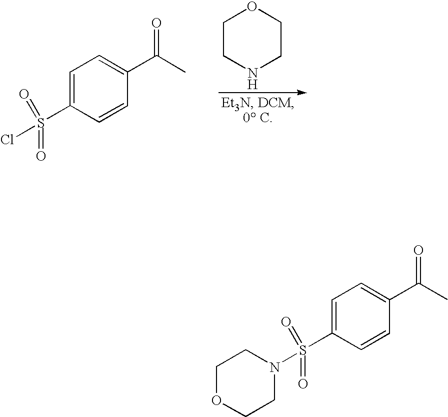

A solution of 4-acetylbenzene sulfonyl chloride (500 mg, 2.3 mmol) in dry dichloromethane (10 mL) was cooled to 0° C. then treated with a solution of morpholine (220 μL, 2.51 mmol, in 5 mL dichloromethane) dropwise. The reaction mixture was then treated with a solution of triethylamine (478 μL, 3.4 mmol in 5 mL dichloromethane) dropwise while maintaining the temperature at 0° C. After the addition was complete the reaction was maintained at 0° C. for 2 h then allowed to warm to room temperature and stirred under an atmosphere of nitrogen overnight. Following this the reaction mixture was concentrated to dryness under reduced pressure. The resultant solid was then diluted with water (20 mL), filtered and washed with a further 50 mL of water then dried to yield the titled 1-(4-(4-morpholinylsulfonyl)phenyl)ethenone as a white solid, (595 mg, 96%).

¹H NMR (300 MHz, DMSO) δ 2.66 (3H, s, CH₃), 2.91 (4H, m, morpholineH), 3.63 (4H, m, morpholineH), 7.88 (2H, d, J=8.7 Hz, ArH), 8.19 (2H, d, J=8.7 Hz, ArH).

MS (ESI⁺) m/z 270 (M+1).

HPLC_{polar(merck)} 99%/0.95 min.

### Synthesis of Side Chains

### Example 7

### Preparation of 4-Bromomethyl-3-iodo-benzoic acid methyl ester

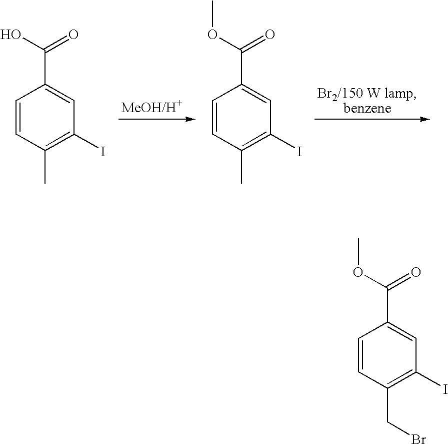

The procedure described in U.S. Pat. No. 4,499,299 was adapted.

### Example 8

### Preparation of 4-Bromomethyl-3-sulfamoyl-benzoic acid methyl ester

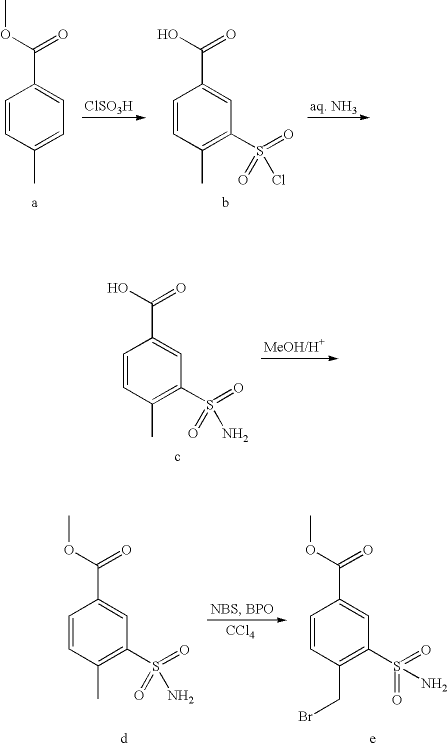

Methyl 4-methylbenzoate (20 g) was heated with chlorosulfonic acid (21 g) at 140° C. for 5 h. The reaction mixture was poured slowly into ice-water and the precipitate was collected, washed by water and dried to give sulfonyl chloride b (9.9 g, 35%).

25% aqueous ammonia (8 mL) was added dropwise into sulfonyl chloride b (2 g) in diethyl ether (40 mL) in ice-bath. The mixture was kept cold for 2 h, then filtered, washed by water and dried to give sulfonylamide c (1.6 g, 90%)

Compound c was esterified as described in U.S. Pat. No. 4,499,299 to give compound d.

Compound d (1.6 g), N-bromosuccinimide (1.77 g) and 0.03 eq of benzoyl peroxide were mixed with carbon tetrachloride (50 mL) and refluxed for 12 h. Flash chromatography (hexane/ethyl aceatate 3:1) afforded compound e (800 mg, 36%)

### Example 9

### Preparation of 2-Amino-4-(toluene-4-sulfonyloxymethyl)-benzoic acid methyl ester

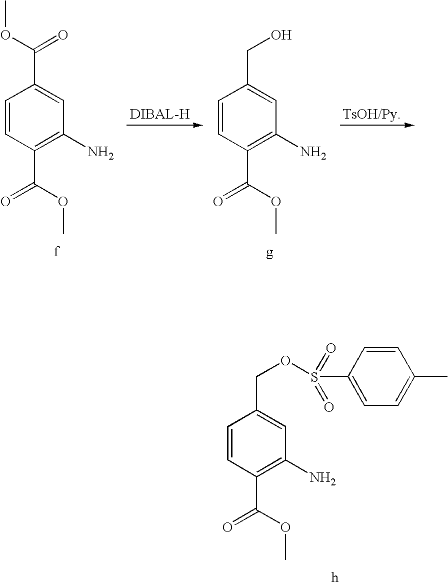

The reactant f was dissolved in tertahydrofuran/diethyl ether (2:1) and treated with DIBAL-H at −78° C. before warming to 0° C. and stirred for further 4 h. The mixture was stirred at room temperature overnight, followed by routine workup to afford the desired product g (33%).

Compound g was refluxed with p-toluenesulfonic acid (1.0 equivalent) in toluene for 2 h to produce the desired compound h (74%)

### Example 10

### Preparation of 5-Bromomethyl-thiophene-2-carboxylic acid methyl ester

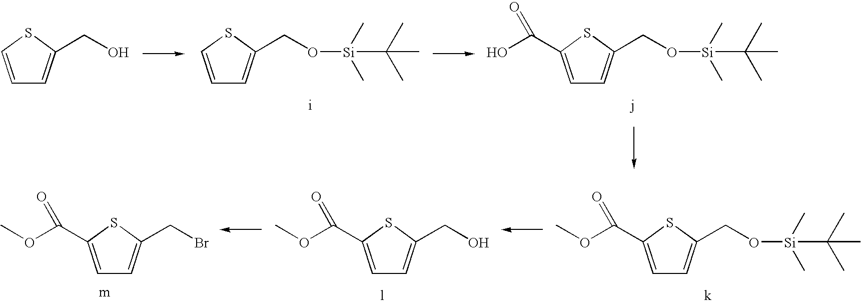

A solution of 2-thiophenemethanol (5 mL, 52.8 mmol) in dichloromethane (100 mL) was treated with t-butyl-dimethyl silyl chloride (11.94 g, 79.2 mmol) followed by diisopropyl ethylamine (18.4 mL, 105.6 mmol) dropwise. The reaction mixture was stirred overnight at room temperature under an atmosphere of nitrogen. Following this the reaction was diluted with a further 50 mL of dichloromethane and the combined organics washed consecutively with water, 0.1M aqueous hydrochloric acid and finally water (3×100 mL each). The combined organics were then dried (MgSO₄) and concentrated under reduced pressure then columned (10% ethyl acetate/petrol) to yield the desired t-butyl-dimethyl-(thiophen-2-ylmethoxy)-silane (i) as a red oil (11.7 g, 97%).

A solution of i (2.0 g, 8.7 mmol) in dry tetrahydrofuran (50 mL) at −40° C. was treated with n-Butyl lithium (1.6M in hexane, 6.6 mL, 10.5 mmol) dropwise and maintained at −40° C. for 1.5 h under a nitrogen atmosphere. Following this CO_{2(g)} (excess) was bubbled through the reaction mixture for 1 hour while allowing the reaction to warm to 0° C. Finally the reaction was quenched by the addition of aqueous ammonium chloride (40 mL), and then extracted into ethyl acetate. The combined organics were then washed with water, dried (MgSO₄) and concentrated under reduced pressure to yield the desired 5-(t-butyl-dimethyl-silanyloxymethyl)-thiophene-2-carboxylic acid (j) as an oil (1.49 g, 62%).

MS (ESI⁻) m/z 271 (M−1).

A solution of j (200 mg, 0.73 mmol) in dichloromethane (30 mL) was treated with diazomethane gas (excess, generated by the basic decomposition of Diazald®). When the reaction was deemed complete the dichloromethane was removed under reduced pressure to afford the desired 5-(t-butyl-dimethyl-silanyloxymethyl)-thiophene-2-carboxylic acid methyl ester (k) as an oil (210 mg, 100%).

A solution of k, (236 mg, 0.82 mmol), in tetrahydrofuran (10 mL) was cooled to 0° C. then treated with tetrabutyl ammonium fluoride (1M in tetrahydrofuran, 1.64 mL, 1.64 mmol) and maintained at 0° C. for 20 min. The reaction was then allowed to warm to room temperature and stirred for a further 2 h. Following this the reaction mixture was poured into brine, (50 mL), and extracted using ethyl acetate (3×50 mL). The combined organics were then dried, (MgSO₄) and concentrated under reduced pressure to yield the desired 5-hydroxymethyl-thiophene-2-carboxylic acid methyl ester (l) as an oil (120 mg, 84%).

To a flask charged with dry dichloromethane (50 mL) was added in order triphenyl phosphine (228 mg, 0.87 mmol), imidazole (59.3 mg, 0.87 mmol) and bromine (44.6 μL, 0.87 mmol). The reaction mixture was then treated with a solution of l (100 mg, 0.58 mmol, in dichloromethane (5 mL)), dropwise and stirred at room temperature under nitrogen for 4 h. When the reaction was deemed complete the mixture was concentrated to dryness under reduced pressure and subjected to flash chromatography (20% ethyl acetate/petrol) to afford the desired 5-bromomethyl-thiophene-2-carboxylic acid methyl ester (m) as colourless crystals, (115 mg, 84%).

¹H NMR (300 MHz, CDCl₃) δ 3.89 (3H, s, CH₃), 4.67 (2H, s, CH₂), 7.10 (1H, m, thienylH), 7.64 (1H, d, J=3.6 Hz, thienylH).

HPLC_{method2} 92%/1.14 min.

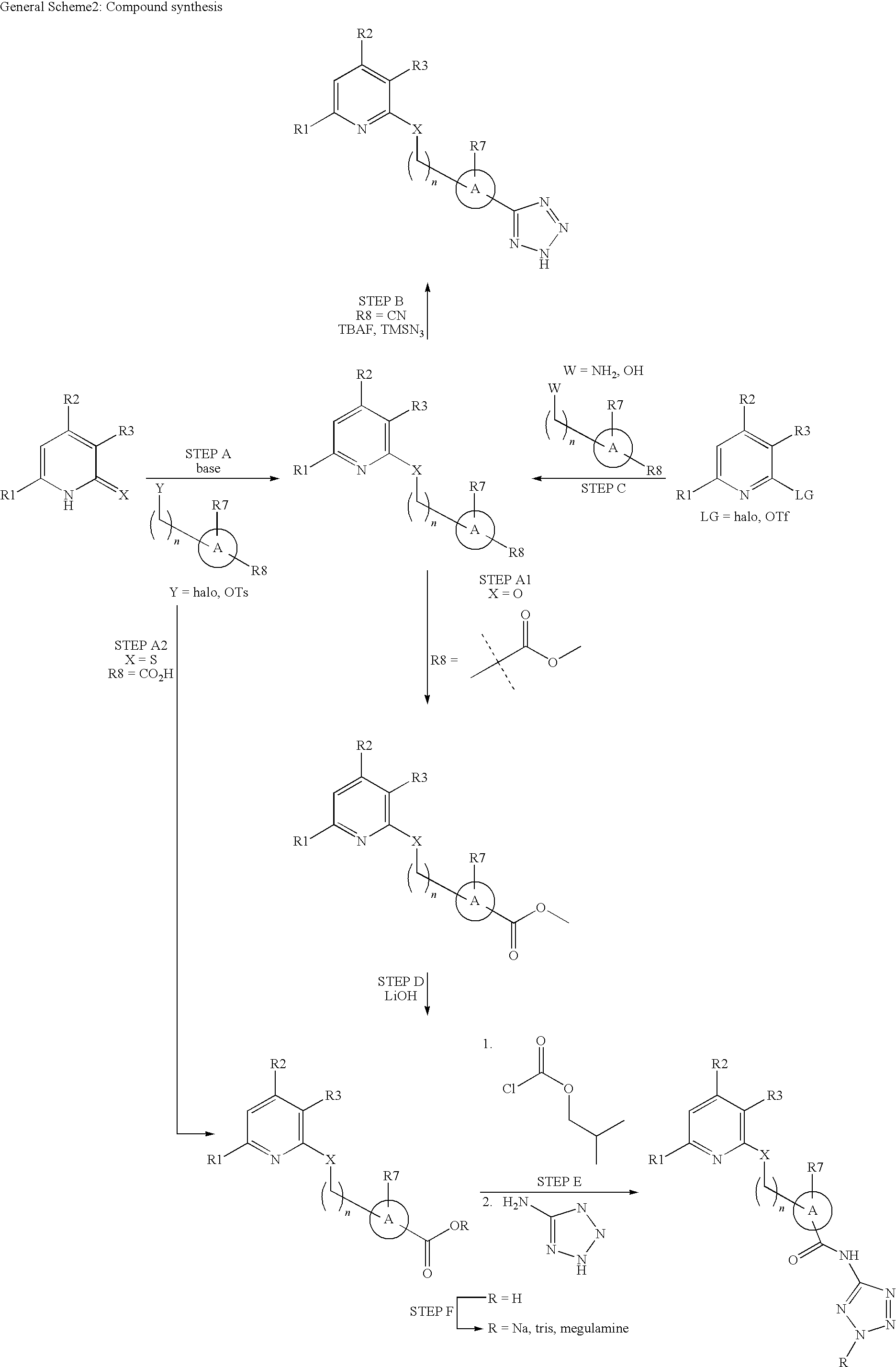

### Example 11

### Preparation of 4-(3-Cyano-4-furan-3-yl-6-thiophen-2-yl-pyridin-2-yloxymethyl)-benzoic acid methyl ester (Step A1)

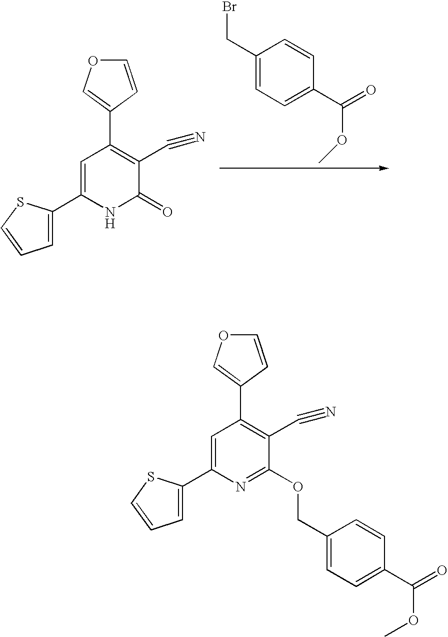

A solution of 4-furan-3-yl-2-oxo-6-thiophen-2-yl-1,2-dihydro-pyridine-3-carbonitrile (53 mg, 1.98 mmol) in dry acetone (5 mL) treated with 4-bromomethyl-benzoic acid methyl ester (500 mg, 2.18 mmol), potassium carbonate (685 mg, 4.96 mmol) and sodium iodide (catalytic, 1%). The reaction mixture was refluxed for 8 h under an atmosphere of nitrogen then allowed to cool to room temperature, giving a milky precipitate. Following this the reaction mixture was diluted with water (10 mL) then filtered to give 4-(3-cyano-4-furan-3-yl-6-thiophen-2-yl-pyridin-2-yloxymethyl)-benzoic acid methyl ester as a cream solid, (770 mg, 93%).

¹H NMR (300 MHz, D⁶DMSO) δ 3.84 (3H, s, Me), 5.66 (2H, s, CH₂), 7.25 (2H, m, thienylH), 7.67 (2H, d, J=8.7 Hz, arylH), 7.83 (1H, dd, J=4.9, 1.2 Hz, furylH), 7.88 (1H, s, ArH), 7.95 (1H, t, J=2.1 Hz, furylH), 7.99 (2H, d, J=8.7 Hz, arylH), 8.09 (1H, dd, J=3.9, 0.9 Hz, thienylH), 8.55 (1H, m, furylH),

MS (ESI⁺) m/z 417 (M+1)

HPLC_{method2} 100%/2.67 min.

By adapting the procedure described in Example 11, the compound of Table 1 were prepared:

TABLE 1
Compound prepared by the procedure of Example 11

| Compound Structure | | ¹H | LC | MS |
|---|---|---|---|---|
| 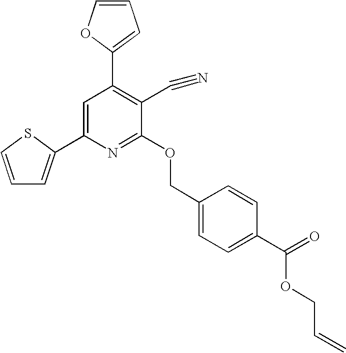 | 4-(3-Cyano-4-furan-2- yl-6-thiophen-2-yl- pyridin-2- yloxymethyl)-benzoic acid allyl ester | y | 3.14(M2) | 443(M + H+) |
| 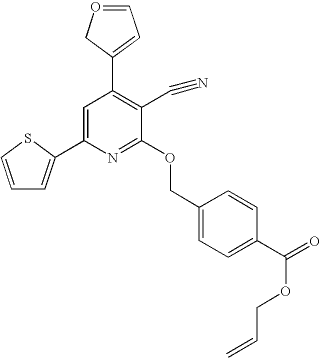 | 4-(3-Cyano-4-furan-3- yl-6-thiophen-2-yl- pyridin-2- yloxymethyl)-benzoic acid allyl ester | y | 2.96(m2) | 443(M + H+) |
| 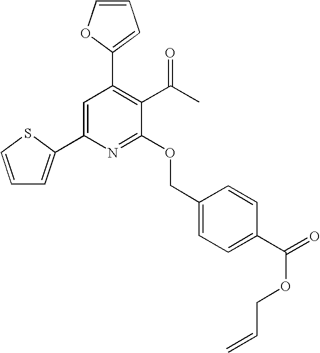 | 4-(3-Acetyl-4-furan-2- yl-6-thiophen-2-yl- pyridin-2- yloxymethyl)-benzoic acid allyl ester | | | |
| 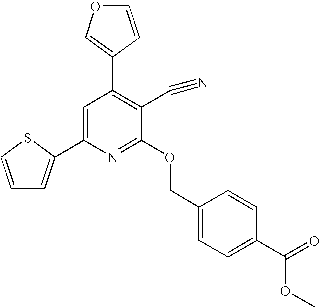 | 4-(3-Cyano-4-furan-3- yl-6-thiophen-2-yl- pyridin-2- yloxymethyl)-benzoic acid methyl ester | Y | 2.75(M2) | 439(M + Na+) |
| 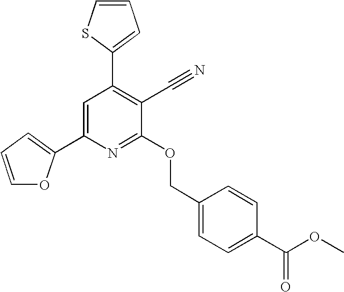 | 4-(3-Cyano-6-furan-2- yl-4-thiophen-2-yl- pyridin-2- yloxymethyl)-benzoic acid methyl ester | | | |
| 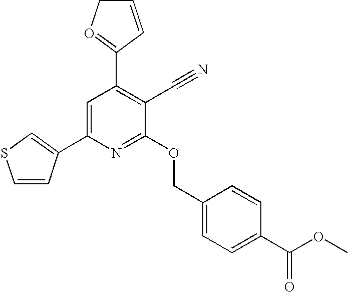 | 4-(3-Cyano-4-furan-3- yl-6-thiophen-3-yl- pyridin-2- yloxymethyl)-benzoic acid methyl ester | y | 2.57(M2) | 417(M + H+) |
| 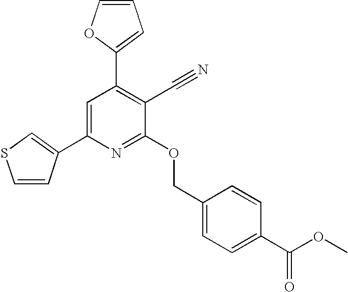 | 4-(3-Cyano-4-furan-2- yl-6-thiophen-3-yl- pyridin-2- yloxymethyl)-benzoic acid methyl ester | y | 2.61(M2) | 417(M + H+) |
| 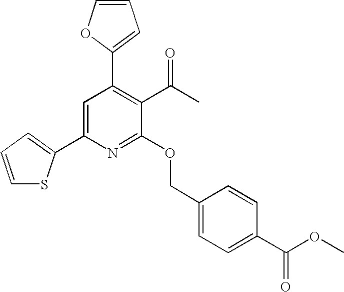 | 4-(3-Acetyl-4-furan-2- yl-6-thiophen-2-yl- pyridin-2- yloxymethyl)-benzoic acid methyl ester | | | |
| 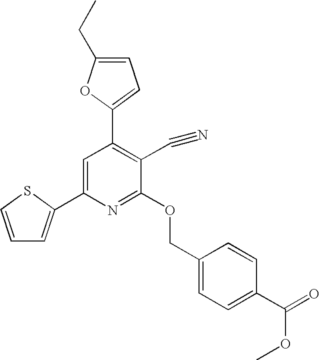 | 4-[3-Cyano-4-(5- ethyl-furan-2-yl)-6- thiophen-2-yl-pyridin- 2-yloxymethyl]- benzoic acid methyl ester | y | 2.76(M2) | 445(M + H+) |
| 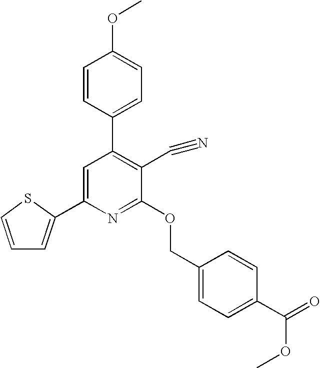 | 4-[3-Cyano-4-(4- methoxy-phenyl)-6- thiophen-2-yl-pyridin- 2-yloxymethyl]- benzoic acid methyl ester | y | 2.61(M2) | 457(M + H+) |
| 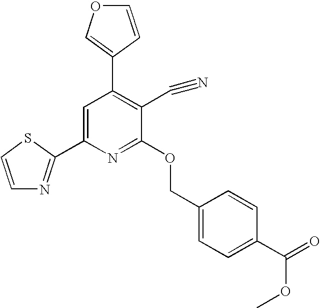 | 4-(3-Cyano-4-furan-3- yl-6-thiazol-2-yl- pyridin-2- yloxymethyl)-benzoic acid methyl ester | y | 2.41(M2) | 418(M + H+) |
| 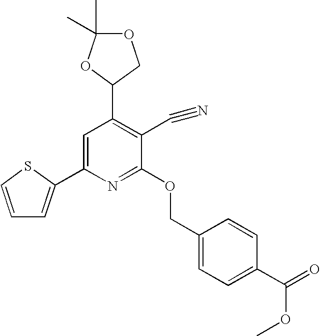 | 4-[3-Cyano-4-(2,2- dimethyl- [1,3]dioxolan-4-yl)-6- thiophen-2-yl-pyridin- 2-yloxymethyl]- benzoic acid methyl ester | y | 2.47(m2) | 451(M + H+) |
| 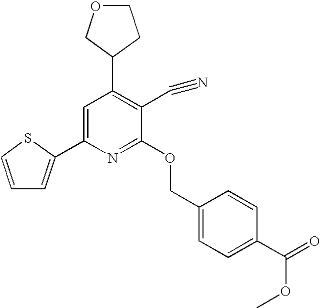 | 4-[3-Cyano-4- (tetrahydro-furan-3- yl)-6-thiophen-2-yl- pyridin-2- yloxymethyl]-benzoic acid methyl ester | y | 2.35(M2) | 421(M + H+) |
| 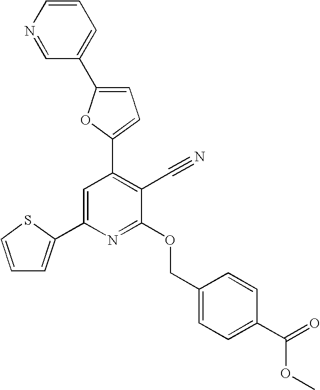 | 4-[3-Cyano-4-(5- pyridin-3-yl-furan-2- yl)-6-thiophen-2-yl- pyridin-2- yloxymethyl]-benzoic acid methyl ester | y | 2.51(M2) | 494(M + H+) |
| 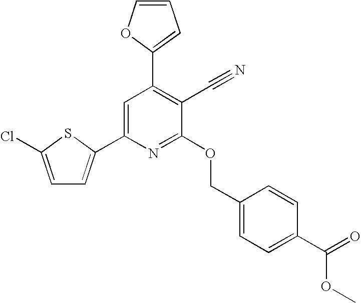 | 4-[6-(5-Chloro- thiophen-2-yl)-3- cyano-4-furan-2-yl- pyridin-2- yloxymethyl]-benzoic acid methyl ester | y | 2.66(M2) | 451(M + H+) |
| 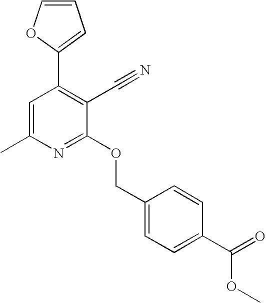 | 4-(3-Cyano-4-furan-2- yl-6-methyl-pyridin-2- yloxymethyl)-benzoic acid methyl ester | y | 2.20(M2) | 349(M + H+) |
| 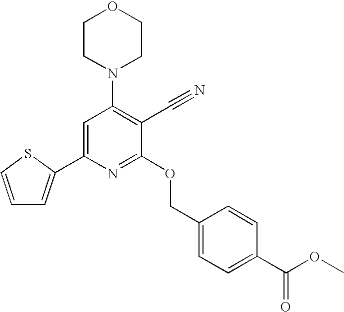 | 4-(3-Cyano-4- morpholin-4-yl-6- thiophen-2-yl-pyridin- 2-yloxymethyl)- benzoic acid methyl ester | | | |
| 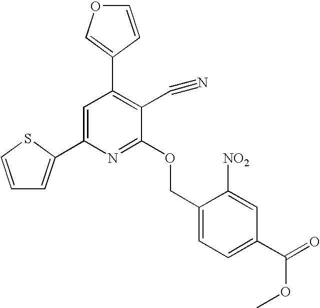 | 4-(3-Cyano-4-furan-3- yl-6-thiophen-2-yl- pyridin-2- yloxymethyl)-3-nitro- benzoic acid methyl ester | y | 2.36(M2) | 460(M − H+) |
| 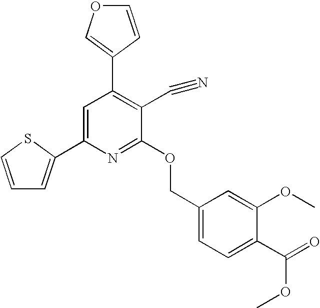 | 4-(3-Cyano-4-furan-3- yl-6-thiophen-2-yl- pyridin-2- yloxymethyl)-2- methoxy-benzoic acid methyl ester | y | 2.25(M2) | 447(M + H+) |
| 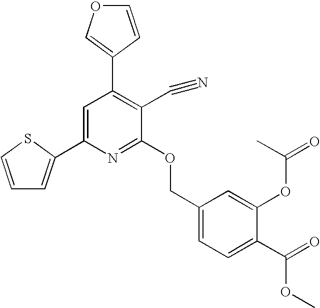 | 2-Acetoxy-4-(3- cyano-4-furan-3-yl-6- thiophen-2-yl-pyridin- 2-yloxymethyl)- benzoic acid methyl ester | | | |
| 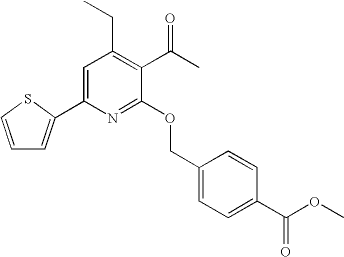 | 4-(3-Acetyl-4-ethyl-6- thiophen-2-yl-pyridin- 2-yloxymethyl)- benzoic acid methyl ester | | | |
| 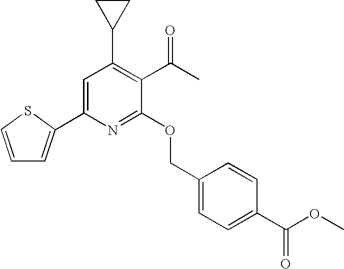 | 4-(3-Acetyl-4- cyclopropyl-6- thiophen-2-yl-pyridin- 2-yloxymethyl)- benzoic acid methyl ester | | | |
| 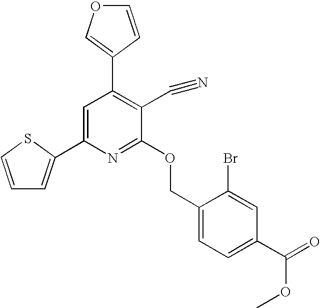 | 3-Bromo-4-(3-cyano- 4-furan-3-yl-6- thiophen-2-yl-pyridin- 2-yloxymethyl)- benzoic acid methyl ester | y | 2.44(M2) | 496(M + H+) |
| 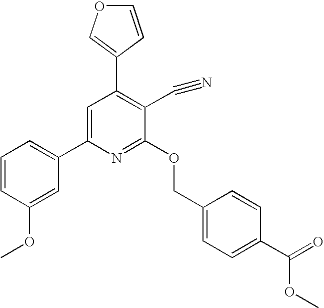 | 4-[3-Cyano-4-furan-3- yl-6-(3-methoxy- phenyl)-pyridin-2- yloxymethyl]-benzoic acid methyl ester | y | | 441(M + H+) |
| 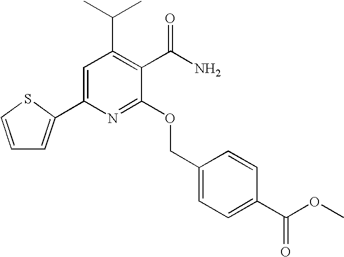 | 4-(3-Carbamoyl-4- isopropyl-6-thiophen- 2-yl-pyridin-2- yloxymethyl)-benzoic acid methyl ester | | | |
| 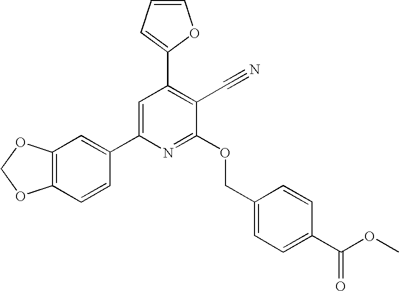 | 4-(6- Benzo[1,3]dioxol-5- yl-3-cyano-4-furan-2- yl-pyridin-2- yloxymethyl)-benzoic acid methyl ester | y | 2.26(M2) | 455(M + H+) |
| 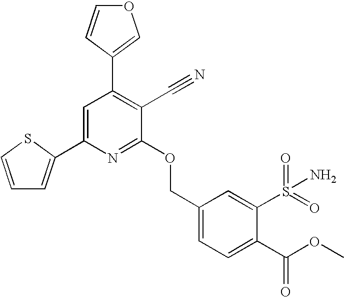 | 4-(3-Cyano-4-furan-3- yl-6-thiophen-2-yl- pyridin-2- yloxymethyl)-2- sulfamoyl-benzoic acid methyl ester | y | 2.37(M2) | 496(M + H+) |
| 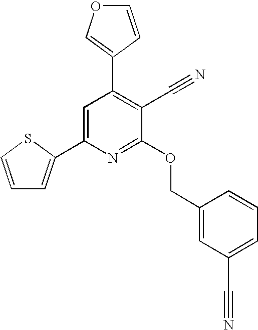 | 2-(3-Cyano- benzyloxy)-4-furan-3- yl-6-thiophen-2-yl- nicotinonitrile | | | |
| 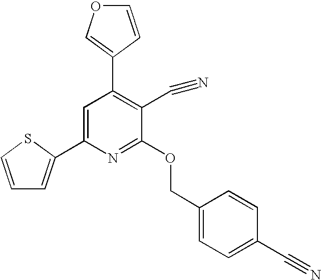 | 2-(4-Cyano- benzyloxy)-4-furan-3- yl-6-thiophen-2-yl- nicotinonitrile | | | |
| 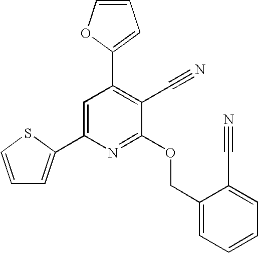 | 2-(2-Cyano- benzyloxy)-4-furan-2- yl-6-thiophen-2-yl- nicotinonitrile | | | |

### Example 12

### Preparation of 4-(3-Cyano-4,6-diphenyl-pyridin-2-ylsulfanylmethyl)-benzoic acid (Step A2)

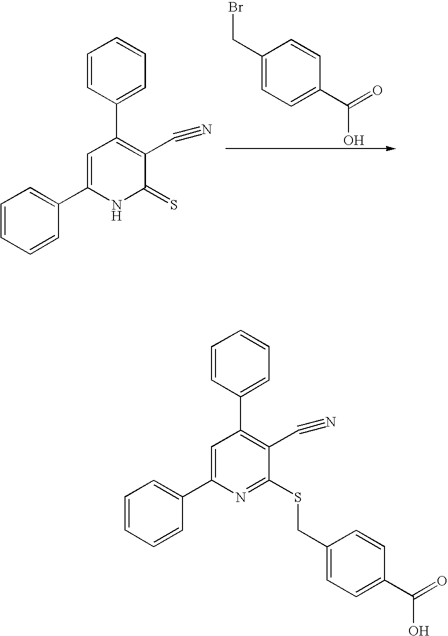

A solution of 4,6-diphenyl-2-thioxo-1,2-dihydro-3-pyridinecarbonitrile (122 mg, 0.42 mmol) in DMF (2 mL) was treated with aqueous potassium hydroxide (1M, 1.05 mL, 1.05 mmol) and allowed to stir for 10 min. Then, 4-bromomethyl-benzoic acid (100 mg, 0.46 mmol) was added and the mixture was allowed to stir overnight. Treatment of the resultant clear solution with 1M HCl (1 mL) afforded a milky precipitate which was filtered to give 4-(3-Cyano-4,6-diphenyl-pyridin-2-ylsulfanylmethyl)-benzoic acid as a tan solid (165 mg, 92%).

¹H NMR (300 MHz, D⁶DMSO) δ 4.78 (2H, s, CH₂), 7.52-7.63 (8H, m, ArH), 7.73-7.76 (2H, m, ArH), 7.87 (2H, d, J=8.7 Hz, ArH), 7.92 (1H, s, ArH), 8.22-8.25 (2H, m, ArH);

MS (ESI⁺) m/z 423. (M+1); MS (ESI⁻) m/z 421 (M−1).

HPLC_{method2} 98%/2.45 min.

### Example 13

### Preparation of 4-Furan-3-yl-2-[4-(2H-tetrazol-5-yl)-benzylamino]-6-thiophen-2-yl-nicotinonitrile (Step B)

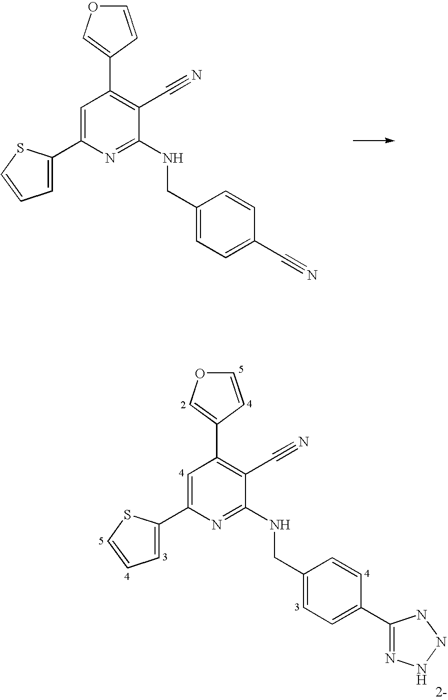

(4-Cyano-benzylamino)-4-furan-3-yl-6-thiophen-2-yl-nicotinonitrile (140 mg, 0.37 mmol), tetrabutylammonium fluoride hydrate (48 mg, 0.183 mmol), trimethylsilyl azide (73 uL, 0.55 mmol) and DMF (1 mL) were combined with stirring in a sealed pressure tube and heated to 90° C. for 36 h. After cooling to room temperature the reaction was diluted with ethyl acetate (20 mL) and washed with aqueous hydrochloric acid (1.0 M, 20 mL). The organic phase was dried (Na₂SO₄), filtered and concentrated in vacuo to afford an orange solid (182 mg) which was subjected to flash chromatography (20% ethyl acetate/hexane to 100% ethyl acetate) to afford 4-furan-3-yl-2-[4-(2H-tetrazol-5-yl)-benzylamino]-6-thiophen-2-yl-nicotinonitrile as an orange solid (29 mg, 19%).

¹H NMR (300 MHz, D⁶DMSO) δ 4.70 (d, J=6.0 Hz, 2H, NHCH₂), 7.15 (m, 1H, H4-furan), 7.17 (dd, J=5.0, 3.7 Hz, 1H, H4-thiophene), 7.40 (s, 1H, H5-pyridine), 7.66 (d, J=8.3 Hz, 2H, H3-aromatic), 7.71 (dd, J=5.0, 0.9 Hz, 1H, H3-thiophene), 7.89 (t, J=1.8 Hz, 1H, H5-furan), 7.93 (dd, J=3.7, 0.9 Hz, 1H, H5-thiophene), 7.96 (d, J=8.3 Hz, 2H, H4-aromatic), 8.44 (t, J=1.8 Hz, H2-furan).

HPLC_{method 2} 92.2%/1.97 min

MS (ESI⁺) m/z 426 (M+1); MS (ESI⁻) m/z 424 (M−1)

By adapting the procedure described in Example 13, the compounds of Table 2 were prepared:

TABLE 2
Compound prepared by the procedure of Example 13

| | Compound | | | |
|---|---|---|---|---|
| Compound Structrue | Name | ¹H | LC | MS |
| 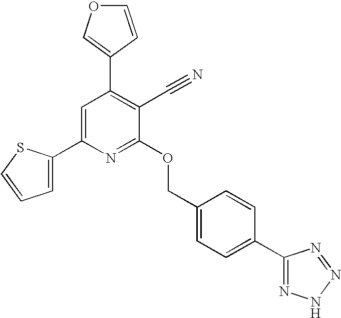 | 4-Furan-3-yl- 2-[4-(2H- tetrazol-5-yl)- benzyloxy]-6- thiophen-2-yl- nicotinonitrile | | | |
| 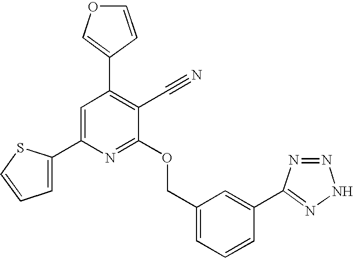 | 4-Furan-3-yl- 2-[3-(2H- tetrazol-5-yl)- benzyloxy]-6- thiophen-2-yl- nicotinonitrile | y | 2.12 (M2) | 427(M + H+) |
| 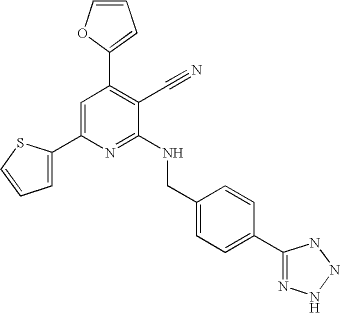 | 4-Furan-2-yl- 2-[4-(2H- tetrazol-5-yl)- benzylamino]- 6-thiophen-2- yl- nicotinonitrile | | | |

### Example 14

### Preparation of 4-[(3-Cyano-4-furan-3-yl-6-thiophen-2-yl-pyridin-2-ylamino)-methyl]-benzoic acid methyl ester (Step C)

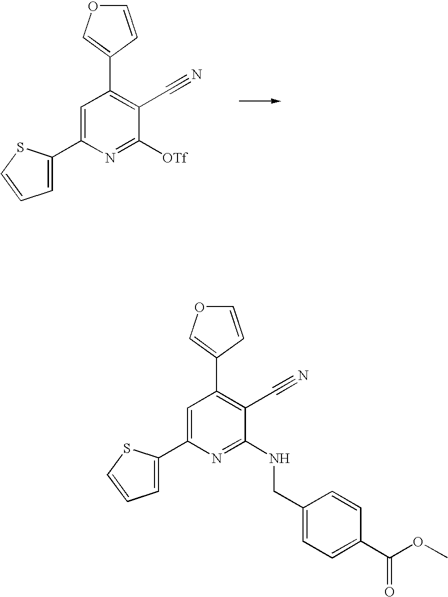

Trifluoro-methanesulfonic acid 3-cyano-4-furan-3-yl-6-thiophen-2-yl-pyridin-2-yl ester (80 mg, 0.20 mmol) was placed into reaction flask and dissolved with dry DMF (10 mL). Triethylamine (111 μL, 0.79 mmol) was added followed by methyl 4-(aminomethyl)-benzoate HCl (81 mg, 0.40 mmol). The reaction mixture was stirred at 80° C. After 4 h of reaction time, the reaction was quenched by addition of water (10 mL). A light orange solid was formed which was collected by filtration and suction dried and obtained a dark cream solid (72.5 mg, 87%).

HPLC_{method2} 91.7%/2.62 min.

MS (ESI⁺) m/z 416 (M+1).

¹H NMR (CDCl₃, 300 MHz) 8.06 (app t, H2 of furan), 7.92 (app d, J_{AA′BB′}8.7 Hz, 2xArH next to COOMe), 7.59 (app d, J=3.9 Hz, H5 of thiophene), 7.48 (t, J=1.8 Hz, H5 of furan), 7.43 (app d, J_{AA′BB}, 8.4 Hz, 2xArH next to CH₂NH), 7.37 (dd, J=5.1 Hz, 1.5 Hz, H4 of furan), 7.03 (dd, J=5.1 Hz, 3.6 Hz, H4 of thiophene), 6.99 (s, 1H of pyridine), 6.80 (app d, J=2.4 Hz, H2 of thiophene), 4.77 (s, CH₂), 3.80 (s, CH₃).

By adapting the procedure described in Example 14, the compound of Table 3 were prepared:

TABLE 3
Compounds prepared by the procedure of Example 14

| Compound Structrue | Compound Name | ¹H | LC | MS |
|---|---|---|---|---|
| 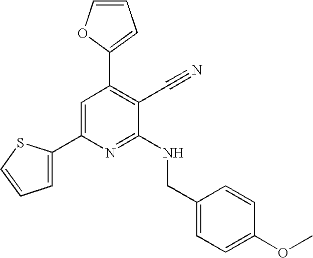 | 4-Furan-2-yl-2-(4- methoxy-benzylamino)- 6-thiophen-2-yl- nicotinonitrile | | | |
| 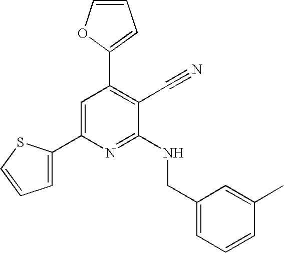 | 4-Furan-2-yl-2-(3- methyl-benzylamino)- 6-thiophen-2-yl- nicotinonitrile | | | |
| 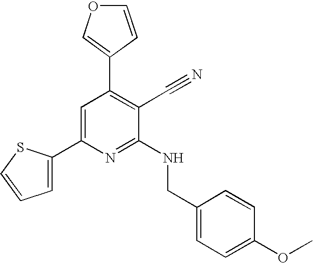 | 4-Furan-3-yl-2-(4- methoxy-benzylamino)- 6-thiophen-2-yl- nicotinonitrile | | | |
| 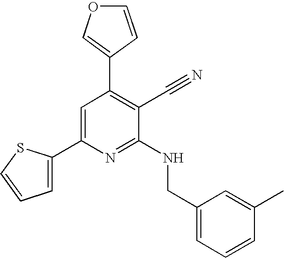 | 4-Furan-3-yl-2-(3- methyl-benzylamino)- 6-thiophen-2-yl- nicotinonitrile | | | |
| 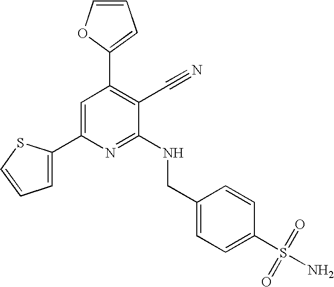 | 4-[(3-Cyano-4-furan-2- yl-6-thiophen-2-yl- pyridin-2-ylamino)- m ethyl]- benzenesulfonamide | | | |
| 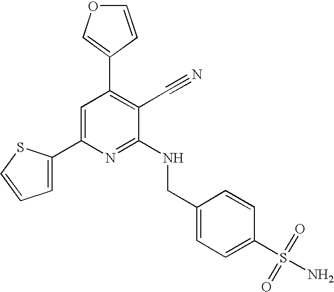 | 4-[(3-Cyano-4-furan-3- yl-6-thiophen-2-yl- pyridin-2-ylamino)- methyl]- benzenesulfonamide | | | |
| 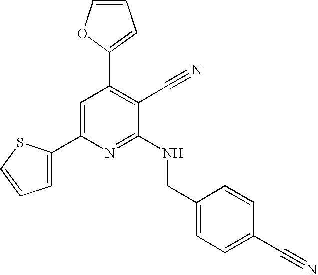 | 2-(4-Cyano- benzylamino)-4-furan- 2-yl-6-thiophen-2-yl- nicotinonitrile | | | |
| 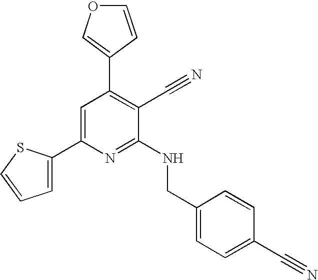 | 2-(4-Cyano- benzylamino)-4-furan- 3-yl-6-thiophen-2-yl- nicotinonitrile | | | |
| 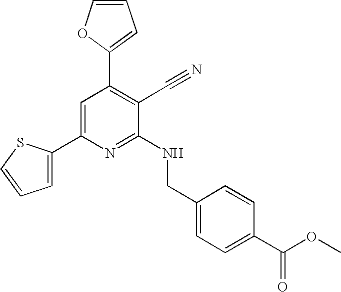 | 4-[(3-Cyano-4-furan-2- yl-6-thiophen-2-yl- pyridin-2-ylamino)- methyl]-benzoic acid methyl ester | | | |

### Example 15

### Preparation of 4-(3-Cyano-4-furan-3-yl-6-thiophen-2-yl-pyridin-2-yloxy)-benzoic acid methyl ester (Step C)

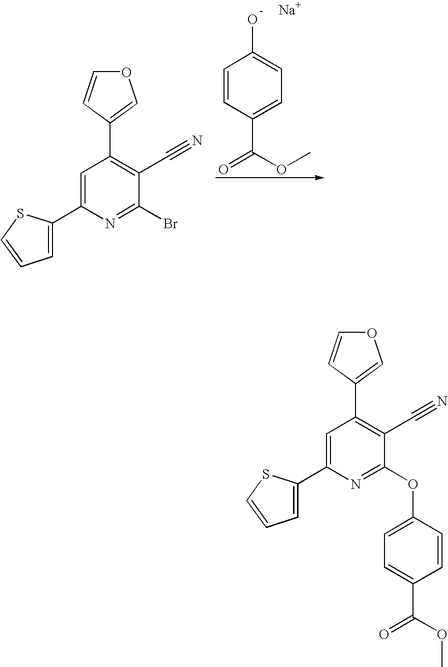

2-Bromo-4-furan-3-yl-6-thiophen-2-yl-nicotinonitrile (160 mg, 0.513 mmol) and sodium; 4-methoxycarbonyl-phenolate (89 mg, 0.513 mmol) in DMF (2 mL) were stirred ay 90° C. for 18 h then cooled to room temperature. The mixture was diluted with aqueous hydrochloric acid (2.0 M, 5 mL) and stirred for 15 min. The resulting precipitate was collected by filtration then subjected to flash chromatography (50% chloroform/hexane) to afford 4-(3-cyano-4-furan-3-yl-6-thiophen-2-yl-pyridin-2-yloxy)-benzoic acid methyl ester as a white solid (141 mg, 68%).

¹H NMR (300 MHz, CDCl₃) δ 3.95 (s, 3H, OCH₃), 6.97 (dd, J=2.3, 0.9 Hz, 1H, H4-furan), 7.08 (dd, J=5.0, 3.7 Hz, 1H, H4-thiophene), 7.37 (d, J=8.7 Hz, 2H, H2-aromatic), 7.42 (dd, J=5.0, 1.4 Hz, H3-thiophene), 7.43 (s, 1H, H5-pyridine), 7.59 (dd, J=3.7, 1.4 Hz, 1H, H5-thiophene), 7.61 (m, 1H, H2 or H5-furan), 8.14 (d, J=8.7 Hz, 2H, H3-aromatic), 8.31 (m, 1H, H2 or H5-furan).

HPLC_{method} 2 97.6%/2.28 min

MS (ESI⁺) m/z 403 (M+1)

By adapting the procedure described in Example 15, the compound of Table 4 were prepared:

TABLE 4
Compounds prepared by the procedure of Example 15

| Compound Structrue | Compound Name | ¹H | LC | MS |
|---|---|---|---|---|
| 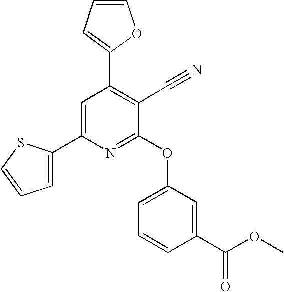 | 3-(3-Cyano-4-furan-2- yl-6-thiophen-2-yl- pyridin-2-yloxy)- benzoic acid methyl ester | | | |
| 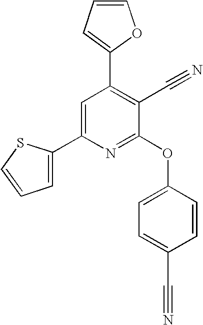 | 2-(4-Cyano-phenoxy)- 4-furan-2-yl-6- thiophen-2-yl- nicotinonitrile | | | |
| 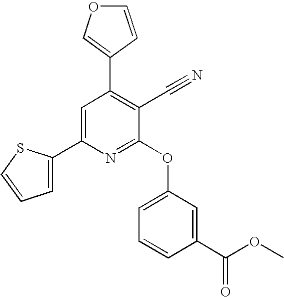 | 3-(3-Cyano-4-furan-3- yl-6-thiophen-2-yl- pyridin-2-yloxy)- benzoic acid methyl ester | | | |
| 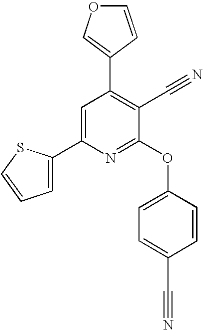 | 2-(4-Cyano-phenoxy)- 4-furan-3-yl-6- thiophen-2-yl- nicotinonitrile | | | |
| 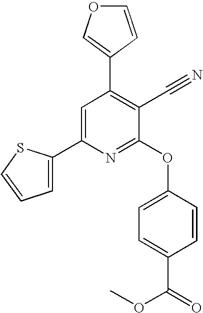 | 4-(3-Cyano-4-furan-3- yl-6-thiophen-2-yl- pyridin-2-yloxy)- benzoic acid methyl ester | | | |

### Example 16

### Preparation of 4-(3-Cyano-4-furan-3-yl-6-thiophen-2-yl-pyridin-2-yloxymethyl)-benzoic acid (Step D)

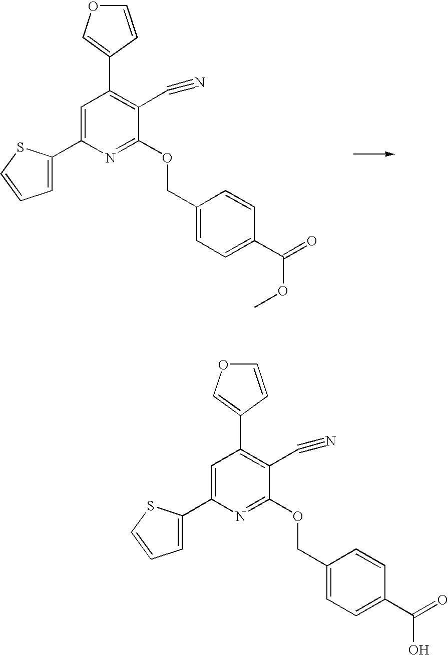

A solution of 4-(3-cyano-4-furan-3-yl-6-thiophen-2-yl-pyridin-2-yloxymethyl)-benzoic acid methyl ester (720 mg, 1.74 mmol) in THF (30 mL) was treated with aqueous lithium hydroxide (1 M, 10.43 mL, 10.43 mmol) and stirred at 50° C. for 8 h then allowed to cool to room temperature. The mixture was diluted with water (5 mL) then made acidic with 1M aqueous hydrochloric acid to give a pale yellow precipitate which was filtered to give 4-(3-cyano-4-furan-3-yl-6-thiophen-2-yl-pyridin-2-yloxymethyl)-benzoic acid as a pale yellow solid, (670 mg, 96%).

¹H NMR (300 MHz, D⁶DMSO) δ 5.65 (2H, s, CH₂), 7.25 (2H, m, thienylH), 7.64 (2H, d, J=8.1 Hz, arylH), 7.83 (1H, d, J=4.8 Hz, furylH), 7.88 (1H, s, ArH), 7.95 (1H, t, J=1.8 Hz, furylH), 7.97 (2H, d, J=8.1 Hz, arylH), 8.10 (1H, d, J=3.9 Hz, thienylH), 8.56 (1H, m, furylH), 12.95 (1H, br s, CO₂H).

MS (ESI⁺) m/z 403 (M+1); MS (ESI⁻) m/z 401(M−1).

HPLC_{method2} 100%/2.45 min.

### Example 17

### Synthesis of 3[3 -cyano-4-furan-3-yl-6-(4-morpholin-4-yl-phenyl)-pyridin-2-yloxymethyl-3-chloro-thiophene-5-carboxylic acid

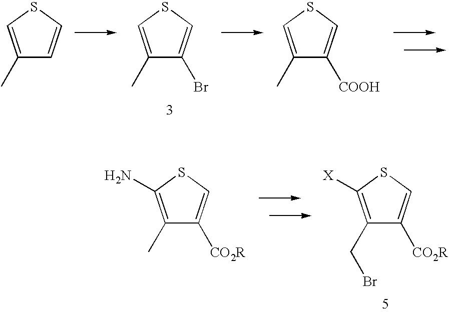

Compound 5,2-substituted, 4-carboxy-(3-Bromomethyl)-thiophenes 6 were prepared by the above route. 3-Bromo-4-methylthiophene 3 was obtained from 3-methylthiophene according to the ref: Syn. Com. 1981; 11(1); 25-28.

### Example 18

### 4-cyano-5-[3-cyano-furan-3-yl-6-(4-morpholin-4-yl-phenyl)-pyridin-2-yloxymethyl]-thiophene-2-carboxylic acid methyl ester

2-Methylthiophene was reacted with bromine (2.0 eq) in acetic acid at rt for 2 h to give the di-bromide 2. Compound 2 was carbonylated with Buli and dry ice to give compound 3 in 74.4% yield and the carboxylate was esterified to give compound 4 in 75.1% yield.

The mixture of compound 4 and CuCN (5.0 eq) was refluxed for 5 hours to afford the cyanide 6 in 51% yield. Bromination gave the desired bromide 7.

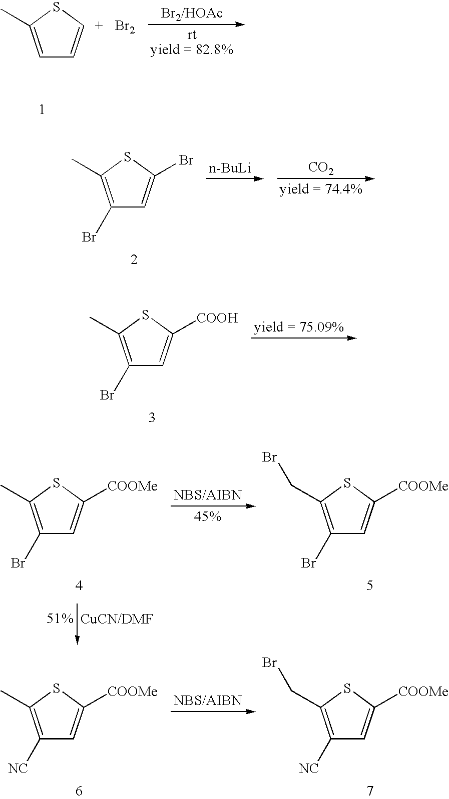

Intermediate 7 was reacted with the 4-furan-3-yl-2-oxo-6-(4-morpholin-4-yl-phenyl)-1,2-dihydro-pyridine-3-carbonitrile by adapting the procedure in example 11 to produce the desired product 4-cyano-5-[3-cyano-furan-3-yl-6-(4-morpholin-4-yl-phenyl)-pyridin-2-yloxymethyl]-thiophene-2-carboxylic acid methyl ester.

### Example 19

### Synthesis of 5-nitro-3-[3-cyano-furan-3-yl-6-(4-morpholin-4-yl-phenyl)-pyridin-2-yl-oxymethyl]-thiophene-2-carboxylic acid and 4-nitro-3-[3-cyano-furan-3-yl-6-(4-morpholin-4-yl-phenyl)-pyridin-2-yl-oxymethyl]-thiophene-2-carboxylic acid

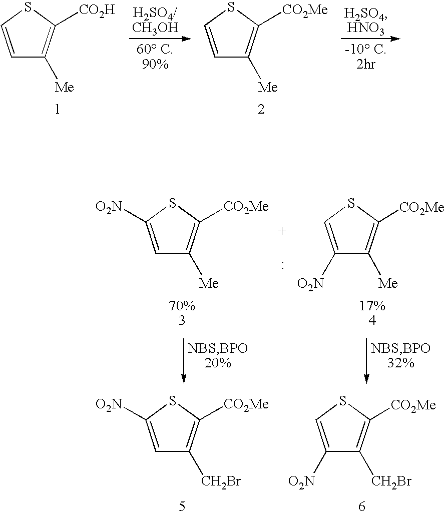

The nitration reaction of 3-methyl-thiophene-2-carboxylic acid methyl ester gave two isomers which were separated by flash chromatography to give 3 (7.8 g, 70% yield) and 4 (1.9 g, 17% yield). Each of them was converted to the bromomethyl compound by NBS/BPO giving 1.0 g of compound 5 and 0.8 g of compound 6 respectively. The side chains 5 and 6 were coupled to the cores by adapting the method as described in example 11 and hydrolysed to the acid by adapting the method in example 16 to give respectively 5-nitro-3-[3-cyano-furan-3-yl-6-(4-morpholin-4-yl-phenyl)-pyridin-2-yl-oxymethyl]-thiophene-2-carboxylic acid (ES-MS 532, M-H+) and its sodium salts (Rt 13.5 min, method 2) and 4-nitro-3-[3-cyano-furan-3-yl-6-(4-morpholin-4-yl-phenyl)-pyridin-2-yl-oxymethyl]-thiophene-2-carboxylic acid.

### Example 20

### Synthesis of ‘3-[3-Cyano-4-furan-3-yl-6-(4-morpholin-4-yl-phenyl)-pyridin-2-yloxymethyl]-5-iodo-thiophene-2-carboxylic acid methyl ester

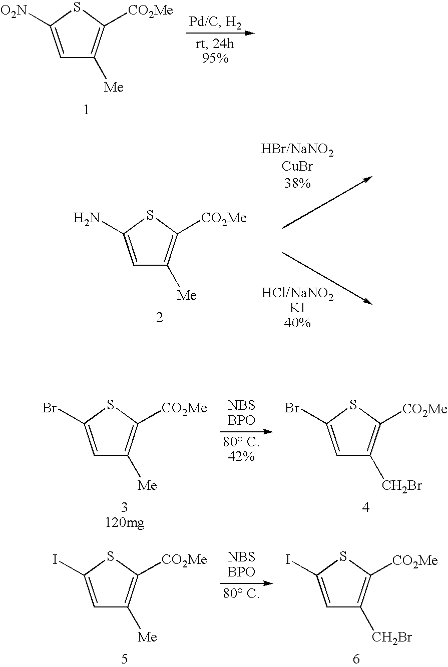

Reduction of compound 3 from example 19 above, gave compound 2. Following which, diazotization reaction gave the bromide 3. Further bromination by NBS/BPO in CCl₄ afforded the new side chain 4.

The iodo side chain 6 was prepared as in the scheme above and coupled to the cores by adapting the method in example 11 to give ‘3-[3-Cyano-4-furan-3-yl-6-(4-morpholin-4-yl-phenyl)-pyridin-2-yloxymethyl]-5-iodo-thiophene-2-carboxylic acid methyl ester (Es-MS, 650, [M+Na+]. The ester was further converted to acid and its sodium salt by adapting the method in example 16 (HPLC Rt 17.0 min, method 2).

### Example 21

### Preparation of Several Aldehydes for Core Formation

### Experiment 21a

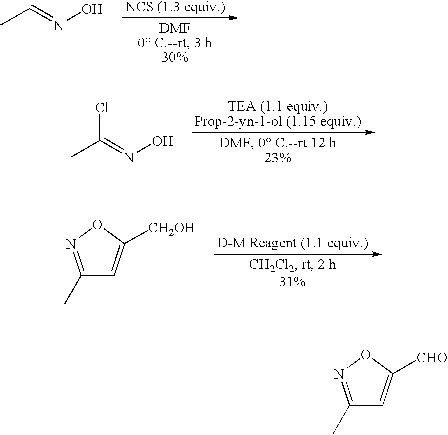

The hydroxymethyl compound was prepared by the literature's method (Ref: J. Antibiot. 1995, 48 (11), 1336-44). It was converted to the corresponding aldehyde (2.8 g, yield 31%) by Dess-Martin oxidation.

### Experiment 21b

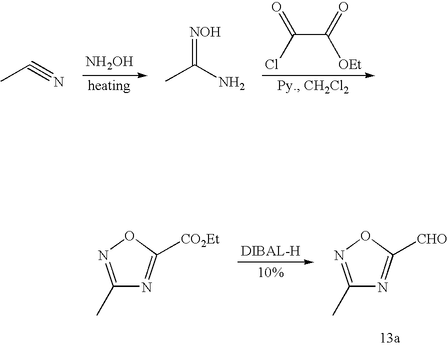

The ethyl ester was prepared according to the ref: J. Med. Chem. 2004, 47 (14), 3642-3657. It was further reduced by DIBAL-H to give the corresponding aldehyde 13a.

### Experiment 21c

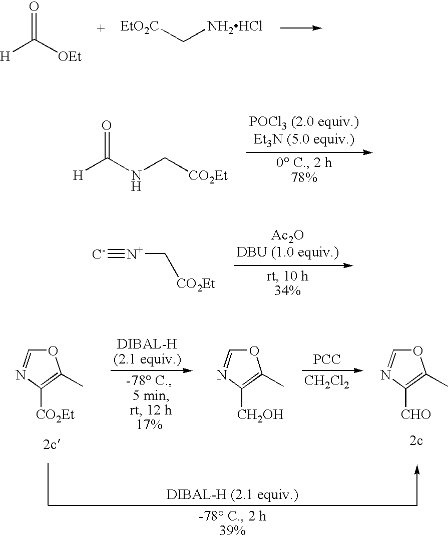

The hydroxyl compound, which was prepared from the DIBAL-H reduction of the ethyl ester 2c′, was oxidized by PCC to give the desired aldehyde 2c in 45% yield

### Experiment 21d

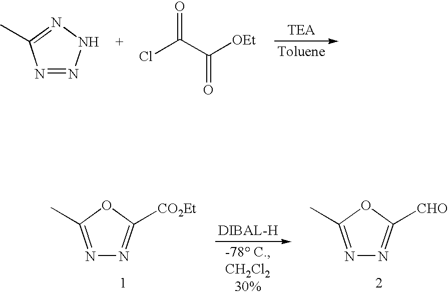

The DIBAL-H reduction of the oxadiazole 1 at −78° C. gave the desired aldehyde 2 in 30% yield.

### Experiment 21e

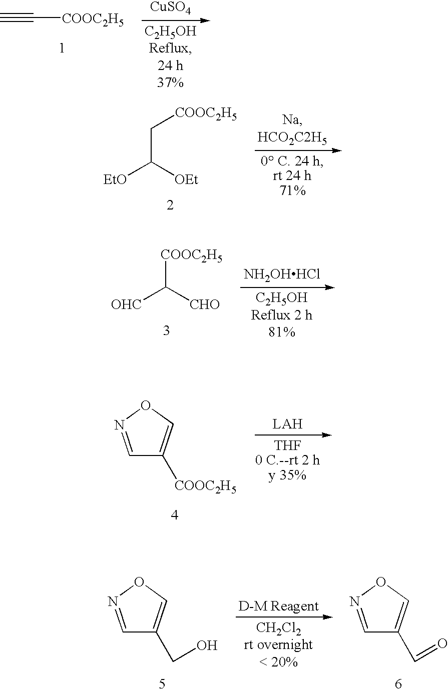

The intermediate 2, was prepared in 37% yield using the method as described in ref.: J. Org. Chem. 1982, 47, 2216-17. The ethyl ester 4 was prepared by the literature method (Ref: Gazz. Chim. Ital. 1947, 77, 206-12). Following lithium aluminium hydride reduction gave the alcohol 5 which was oxides to the aldehyde 6.

### Experiment 21f

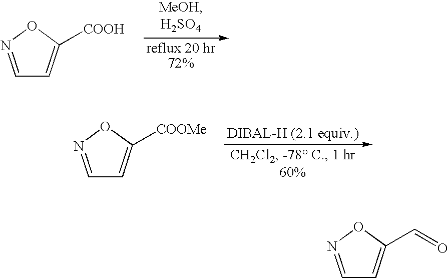

Isoxazole-5-carboxylic acid, which was commercially available from TCI, was converted to the methyl ester, and then reduced by DIBAL-H to afford the isoxazole-5-carbaldehyde (1.05 g, yield 60%).

### Experiment 21g

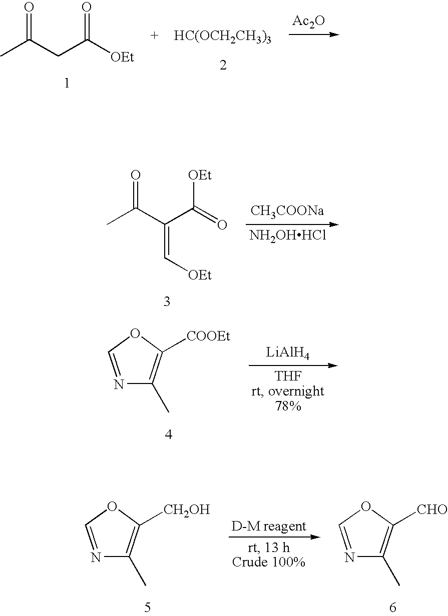

Dess-Martin oxidation of the alcohol 5 afforded the desired aldehyde 6, contaminated by the starting material 4b′. 2 g of the crude aldehyde 5b was obtained and used directly for the core formation.

### Example 21h

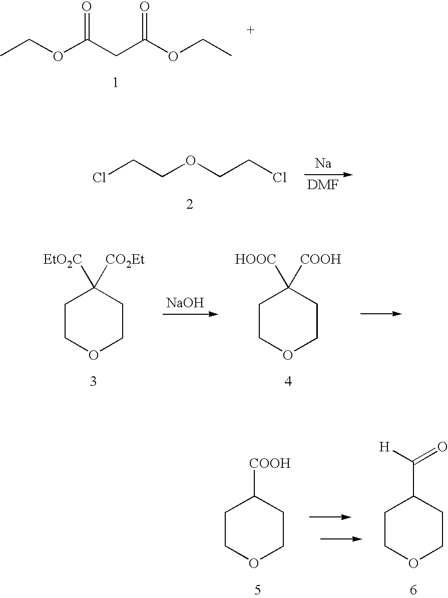

3.2 g of compound 5 was prepared according to the ref: Helv. Chim. Acta. 1997, 80, 1528-1554 and was converted to the corresponding aldehyde 6.

### Experiment 21i

0.5 g of aldehyde 4 was prepared in 6% overall three-step yield. (Ref: J. Med. Chem. 1999, 42, 4961-4969)

### Example 21j

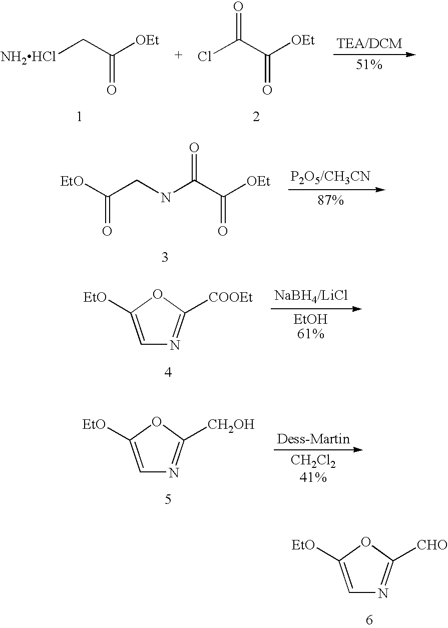

Compound 4 was prepared according to the Ref: J. Hetero. Chem. 1995, 32, 1693-1702. Then, it was reduced by NaBH₄/LiCl in ethanol solution. Further oxidation by Dess-Martin reagent gave the desired aldehyde 6 in 25% overall two-step yield.

### Example 21k

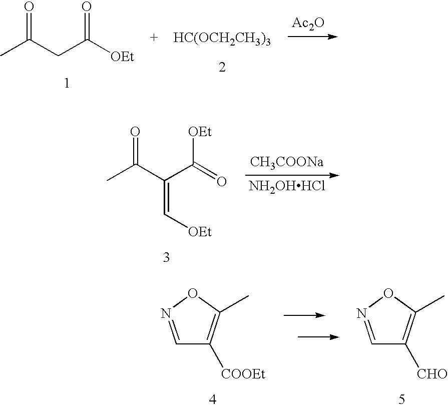

Compound 4 was obtained according to the Ref: J. Chem. Soc. PTI. 1988, 1875-1880. and was converted to the aldehyde 5.

### Example 22

### Preparation of (4-morpholin-4-yl-phenyl)-1,2-dihydro-pyridine-3-carbonitrile cores

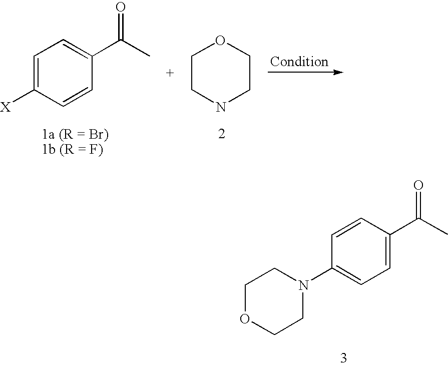

Then, 4-fluorineacetophenone was used as the substrate of SNAR reaction for the preparation of compound 3 (Entry 5-8). Entry 7 gave the best result.

| | | | Reaction | |
|---|---|---|---|---|
| Entry | X | Condition | Time | Result |
| 1 | X = Br | Pddf, Pd(OAc)2/t- | 48 h | Complicated |
| | | BuONa toluene | | |
| 2 | X = Br | Pddf, Pd2(dba)3•CH3Cl | 24 h | No reaction |
| | | t-BuONa, toluene | | |
| 3 | X = Br | CuI L-proline | 24 h | No reaction |
| | | K₃PO₄/DMSO | | |
| 4 | X = Br | NaNH₂ THF | 3 h | Unknown product |
| 5 | X = F | MeCN | 48 h | Y = 17% |
| 6 | X = F | DMSO | 36 h | Y = 50% |
| 7 | X = F | K₂CO₃ DMSO | 48 h | Y = 80% |
| 8 | X = F | Et₃N DMSO | 48 h | Y = 60% |

The (4-morpholino)-acetophenone (1) was then converted to a core (e.g 4) by the method outlined in the scheme below:

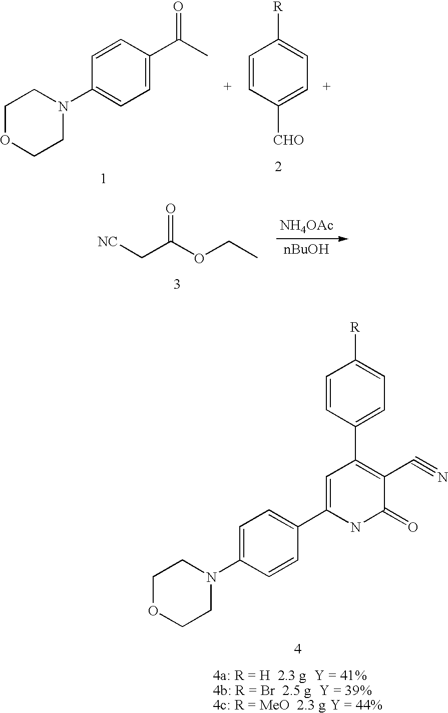

compounds 4a-4-c were prepared by the one-pot reaction.

### Example 23

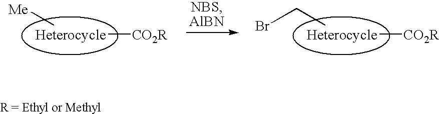

Three side chains were prepared by NBS bromination of methyl group, which were indicated as the following table:

| | | | |
|---|---|---|---|
| Entry | Structure | Yield | Weight |

| | | | |
|---|---|---|---|
| 1 | 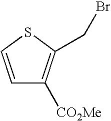 | 49% | 2.62 g |
| 2 | 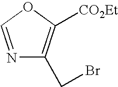 | 59% | 1.8 g |
| 3 | 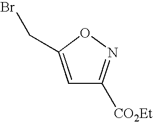 | 42% | 0.12 g |

By adapting the procedure described in Example 16, the compound of Table 5 were prepared:

TABLE 5
Compounds prepared by the procedure of Example 16

| Compound Structure | Compound Name | ¹H | LC | MS |
|---|---|---|---|---|
| 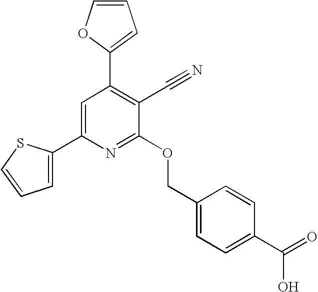 | 4-(3-Cyano-4-furan-2- yl-6-thiophen-2-yl- pyridin-2- yloxymethyl)-benzoic acid | y | 2.34(M2) | 403(M + H+) |
| 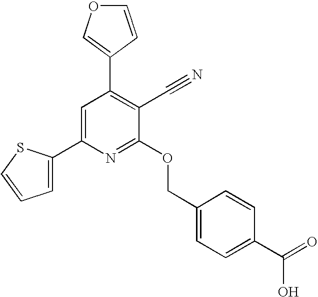 | 4-(3-Cyano-4-furan-3- yl-6-thiophen-2-yl- pyridin-2- yloxymethyl)-benzoic acid | y | 2.31(M2) | 401(M − H+) |
| 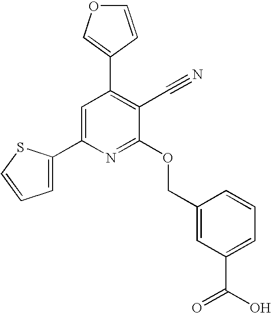 | 3-(3-Cyano-4-furan-3- yl-6-thiophen-2-yl- pyridin-2- yloxymethyl)-benzoic acid | | | |
| 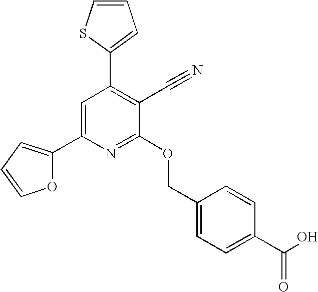 | 4-(3-Cyano-6-furan-2- yl-4-thiophen-2-yl- pyridin-2- yloxymethyl)-benzoic acid | | | |
| 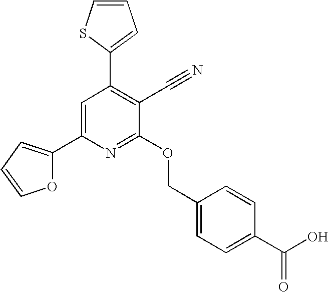 | 4-(3-Cyano-6-furan-2- yl-4-thiophen-2-yl- pyridin-2- yloxymethyl)-benzoic acid | | | |
| 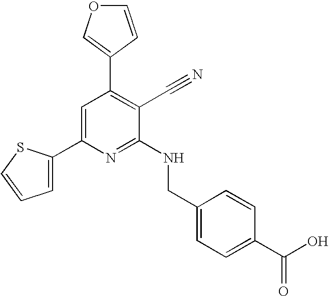 | 4-[(3-Cyano-4-furan-3- yl-6-thiophen-2-yl- pyridin-2-ylamino)- methyl]-benzoic acid | | | |
| 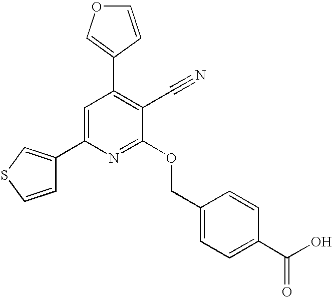 | 4-(3-Cyano-4-furan-3- yl-6-thiophen-3-yl- pyridin-2- yloxymethyl)-benzoic acid | | | |
| 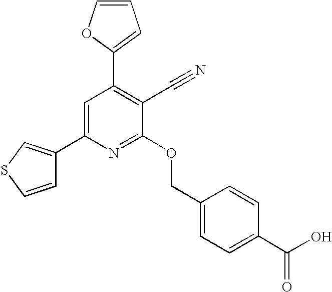 | 4-(3-Cyano-4-furan-2- yl-6-thiophen-3-yl- pyridin-2- yloxymethyl)-benzoic acid | | | |
| 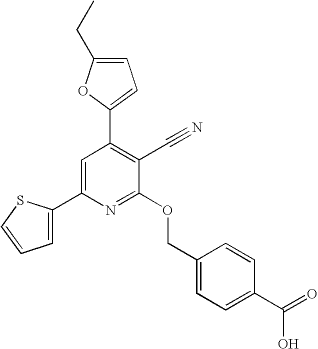 | 4-[3-Cyano-4-(5-ethyl- furan-2-yl)-6-thiophen- 2-yl-pyridin-2- ylxoymethyl]-benzoic acid | | | |
| 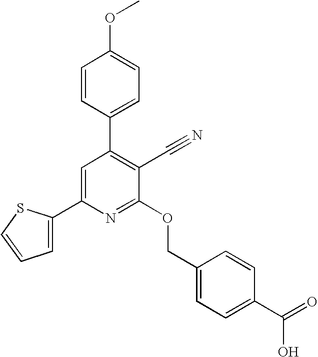 | 4-[3-Cyano-4-(4- methoxy-phenyl)-6- thiophen-2-yl-pyridin- 2-yloxymethyl]-benzoic acid | | | |
| 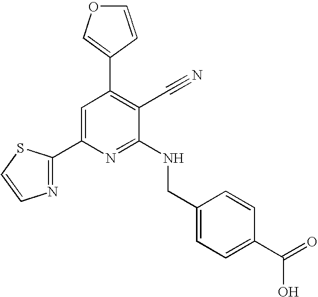 | 4-(3-Cyano-4-furan-3- yl-6-thiazol-2-yl- pyridin-2- yloxymethyl)-benzoic acid | | | |
| 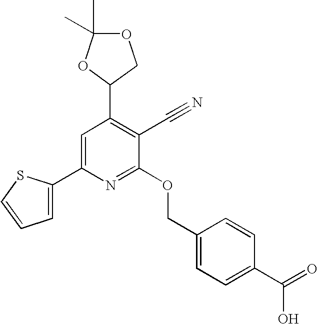 | 4-[3-Cyano-4-(2,2- dimethyl-[1,3]dioxolan- 4-yl)-6-thiophen-2-yl- pyridin-2- yloxymethyl]-benzoic acid | | | |
| 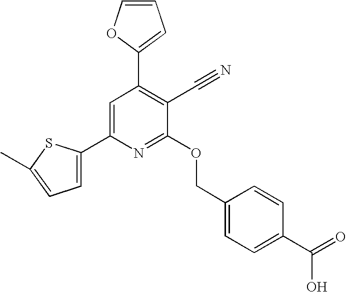 | 4-[3-Cyano-4-furan-2- yl-6-(5-methyl- thiophen-2-yl)-pyridin- 2-yloxymethyl]-benzoic acid | | | |
| 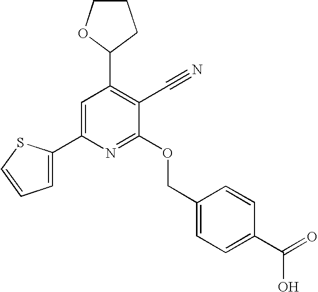 | 4-[3-Cyano-4- (tetrahydro-furan-3-yl)- 6-thiophen-2-yl- pyridin-2- yloxymethyl]-benzoic acid | | | |
| 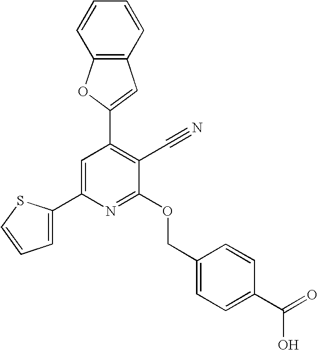 | 4-(4-Benzofuran-2-yl- 3-cyano-6-thiophen-2- yl-pyridin-2- yloxymethyl)-benzoic acid | | | |
| 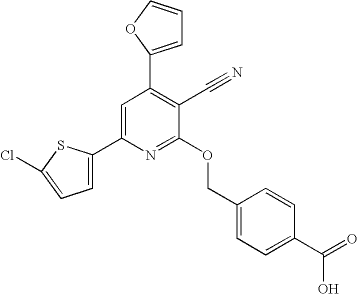 | 4-[6-(5-Chloro- thiophen-2-yl)-3-cyano- 4-furan-2-yl-pyridin-2- yloxymethyl]-benzoic acid | | | |
| 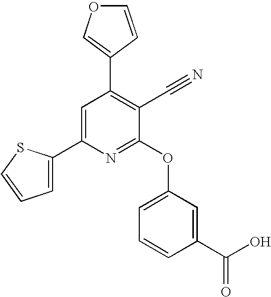 | 3-(3-Cyano-4-furan-3- yl-6-thiophen-2-yl- pyridin-2-yloxy)- benzoic acid | | | |
| 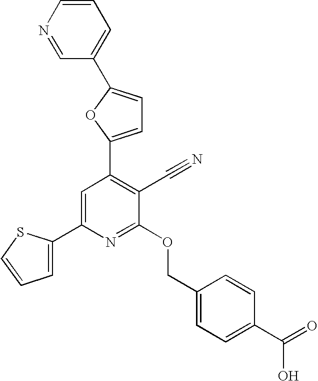 | 4-[3-Cyano-4-(5- pyridin-3-yl-furan-2- yl)-6-thiophen-2-yl- pyridin-2- yloxymethyl]-benzoic acid | | | |
| 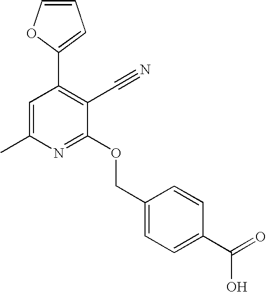 | 4-(3-Cyano-4-furan-2- yl-6-methyl-pyridin-2- yloxymethyl)-benzoic acid | | | |
| 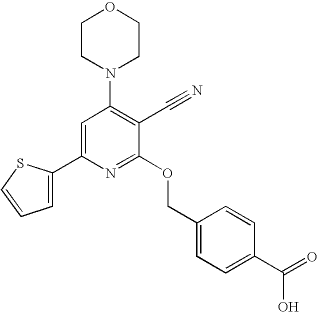 | 4-(3-Cyano-4- morpholin-4-yl-6- thiophen-2-yl-pyridin- 2-yloxymethyl)-benzoic acid | | | |
| 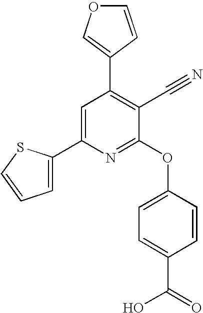 | 4-(3-Cyano-4-furan-3- yl-6-thiophen-2-yl- pyridin-2-yloxy)- benzoic acid | | | |
| 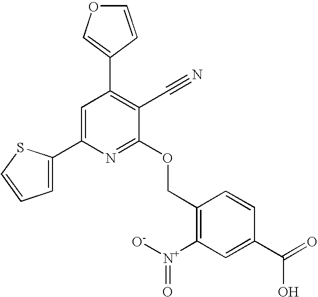 | 4-(3-Cyano-4-furan-3- yl-6-thiophen-2-yl- pyridin-2- yloxymethyl)-3-nitro- benzoic acid | | | |
| 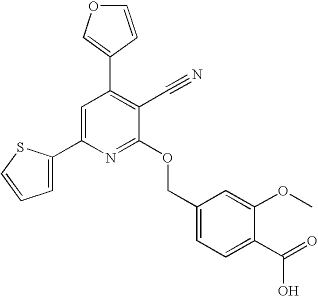 | 4-(3-Cyano-4-furan-3- yl-6-thiophen-2-yl- pyridin-2- yloxymethyl)-2- methoxy-benzoic acid | | | |
| 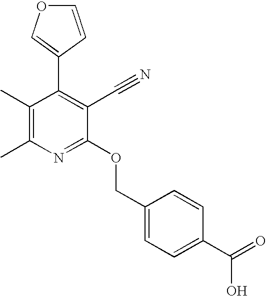 | 4-(3-Cyano-4-furan-3- yl-5,6-dimethyl- pyridin-2- yloxymethyl)-benzoic acid | | | |
| 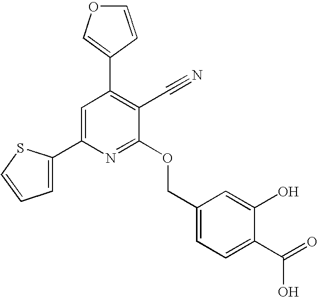 | 4-(3-Cyano-4-furan-3- yl-6-thiophen-2-yl- pyridin-2- yloxymethyl)-2- hydroxy-benzoic acid | | | |
| 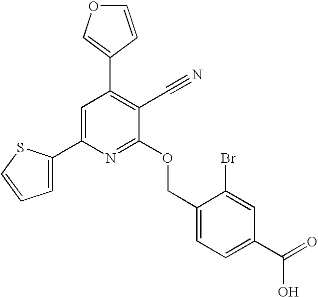 | 3-Bromo-4-(3-cyano-4- furan-3-yl-6-thiophen- 2-yl-pyridin-2- yloxymethyl)-benzoic acid | | | |
| 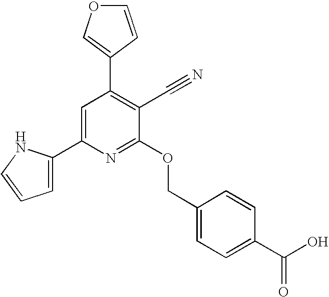 | 4-[3-Cyano-4-furan-3- yl-6-(1H-pyrrol-2-yl)- pyridin-2- yloxymethyl]-benzoic acid | | | |
| 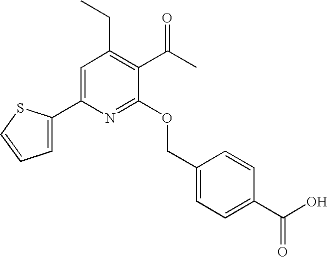 | 4-(3-Acetyl-4-ethyl-6- thiophen-2-yl-pyridin- 2-yloxymethyl)-benzoic acid | | | |
| 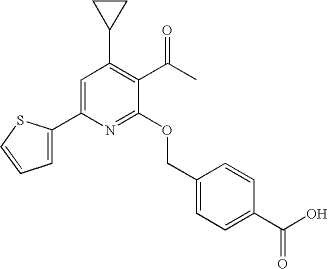 | 4-(3-Acetyl-4- cyclopropyl-6- thiophen-2-yl-pyridin- 2-yloxymethyl)-benzoic acid | | | |
| 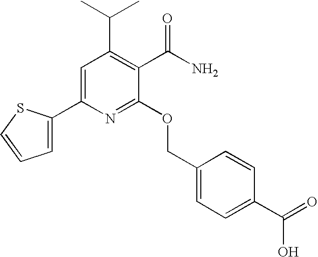 | 4-(3-Carbamoyl-4- isopropyl-6-thiophen-2- yl-pyridin-2- yloxymethyl)-benzoic acid | | | |
| 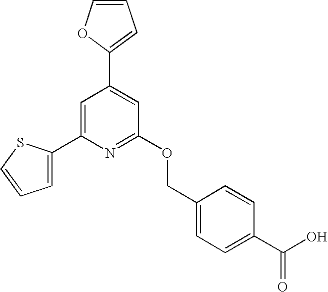 | 4-(4-Furan-2-yl-6- thiophen-2-pyridin- 2-yloxymethyl)-benzoic acid | | | |
| 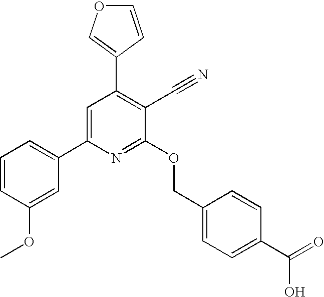 | 4-[3-Cyano-4-furan-3- yl-6-(3-methoxy- phenyl)-pyridin-2- yloxymethyl]-benzoic acid | | | |
| 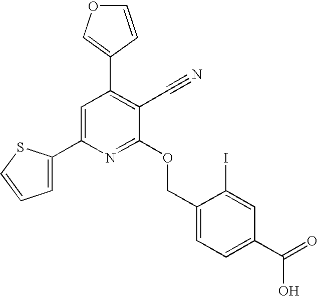 | 4-(3-Cyano-4-furan-3- yl-6-thiophen-2-yl- pyridin-2- yloxymethyl)-3-iodo- benzoic acid | | | |
| 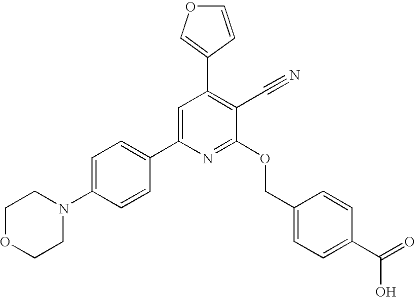 | 4-[3-Cyano-4-furan-3- yl-6-(4-morpholin-4-yl- phenyl)-pyridin-2- yloxymethyl]-benzoic acid | | | |
| 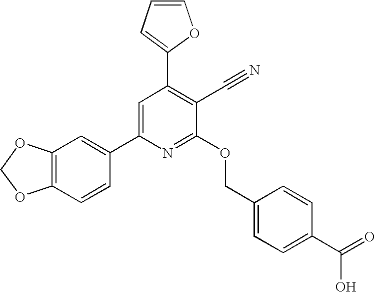 | 4-(6-Benzo[1,3]dioxol- 5-yl-3-cyano-4-furan-2- yl-pyridin-2- yloxymethyl)-benzoic acid | | | |
| 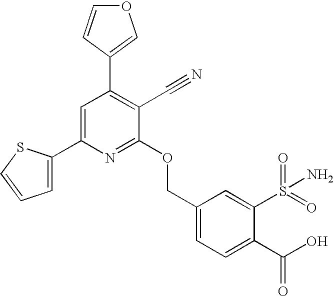 | 4-(3-Cyano-4-furan-3- yl-6-thiophen-2-yl- pyridin-2- yloxymethyl)-2- sulfamoyl-benzoic acid | | | |

### Example 17

### Preparation of 4-(3-Cyano-4-furan-3-yl-6-thiophen-2-yl-pyridin-2-yloxymethyl)-N-(2H-tetrazol-5-yl)-benzamide (Step E)

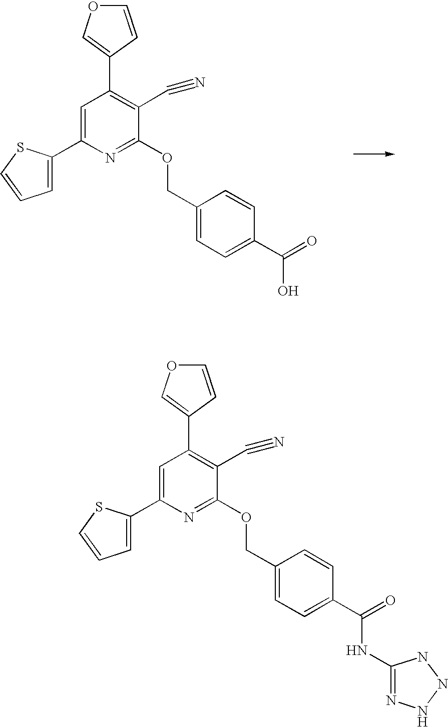

4-(3-Cyano-4-furan-3-yl-6-thiophen-2-yl-pyridin-2-yloxymethyl)-benzoic acid (340 mg, 0.844 mmol) was dissolved with stirring under nitrogen in dry THF (15 mL). Triethylamine (236 μL, 1.69 mmol) was added dropwise and the solution stirred at room temperature for 15 min then cooled to −5° C. (ice/salt/water bath). Isobutyl chloroformate (220 μL, 1.69 mmol) was added dropwise and after 20 min a solution of 5-aminotetrazole (144 mg, 1.69 mmol) in dry DMF (1 mL) was added dropwise over 2 min, followed by Triethylamine (236 μL, 1.69 mmol). The cooling bath was removed and the reaction allowed to warm to room temperature and stirred for 18 h. Volatiles were removed in vacuo and the residue triturated with 1.0 M HCl (20 mL) and water (20 mL). The suspension was sonicated to break up the material then filtered. The residue was triturated with hot methanol, then filtered and washed with 2.0 M NaOH (10 mL), water (10 mL), then dried under vacuum at 50° C. for 2 h to afford 4-(3-cyano-4-furan-3-yl-6-thiophen-2-yl-pyridin-2-yloxymethyl)-N-(2H-tetrazol-5-yl)-benzamide (205 mg, 52%) as a slight brown solid: ¹H NMR (D⁶DMSO) δ 2.0 (s, 2H, CH₂), 7.25 (m, 2H, H4-thiophene and H5-furan), 7.63 (d, J=8.3 Hz, 2H, H3-aromatic), 7.83 (dd, J=5.0, 1.2 Hz, 1H, H3-thiophene), 7.88 (s, 1H, H5-pyridine), 7.94 (m, 1H, H2 or H5-furan), 8.02 (d, J=8.3 Hz, 2H, H4-aromatic), 8.11 (dd, J=3.7, 1.2 Hz, 1H, H5-thiophene), 8.56 (m, 1H, H2 or H5-furan), 10.47 (s, 1H, NH).

MS (ESI⁻) m/z 468 (M−1).

### Example 18

### Preparation of Sodium; 4-(3-cyano-4-furan-3-yl-6-thiophen-2-yl-pyridin-2-yloxymethyl)-benzoate (Step F)

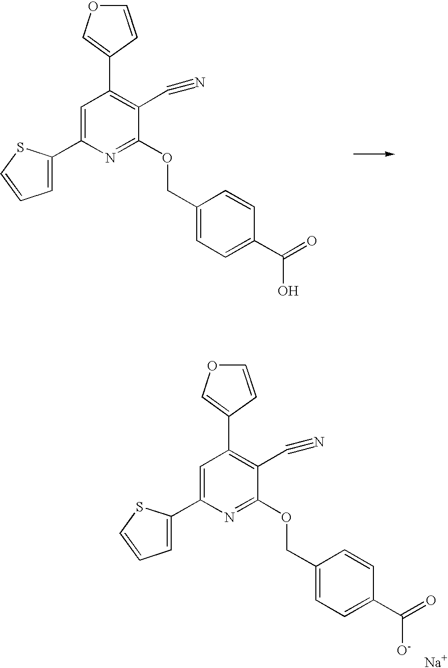

Acid (1 mmol) was dissolved/suspended in methanol (1 mL) with stirring. Aqueous sodium hydroxide solution (2.0 M, 1 mmol) (or aqueous tris solution (2.0 M, 1 mmol) for tris salts) was added and the resulting mixture stirred for 10 min at room temperature. Volatiles were removed in vacuo and the residue dissolved in water (2 mL) filtered (porsity 4 sinter) and freeze dried for 2 d.

TABLE 6
Compounds prepared by the procedure of Example 18

| Compound Structure | Compound Name | ¹H | LC | 0MS |
|---|---|---|---|---|
| 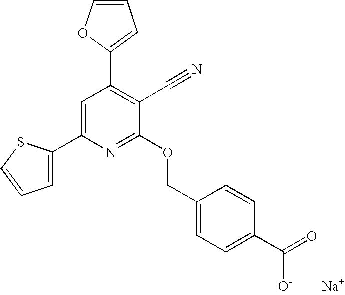 | Sodium; 4-(3-cyano-4- furan-2-yl-6-thiophen- 2-yl-pyridin-2- yloxymethyl)-benzoate | | 2.12 (M2) | 426 (M + H+) |
| 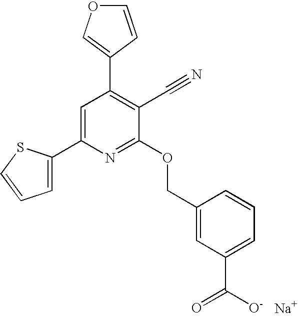 | Sodium; 3-(3-cyano-4- furan-3-yl-6-thiophen- 2-yl-pyridin-2- yloxymethyl)-benzoate | | 2.42 (M2) | |
| 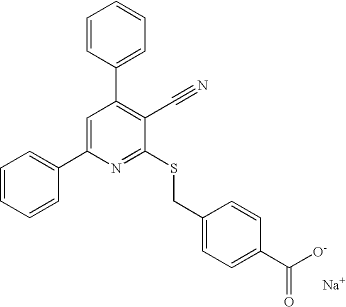 | Sodium; 4-(3-cyano- 4,6-diphenyl-pyridin-2- ylsulfanylmethyl)- benzoate | | | |
| 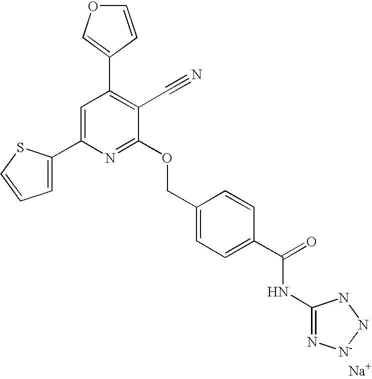 | 4-(3-Cyano-4-furan-3- yl-6-thiophen-2-yl- pyridin-2- yloxymethyl)-N-(2H- tetrazol-5-yl)- benzamide; sodium salt | | 2.14 (M2) | |
| 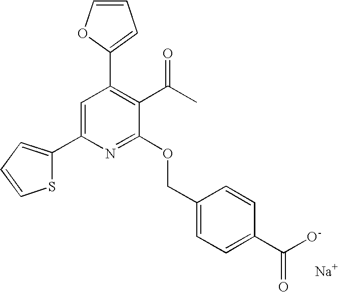 | Sodium; 4-(3-acetyl-4- furan-2-yl-6-thiophen- 2-yl-pyridin-2- yloxymethyl)-benzoate | | | |
| 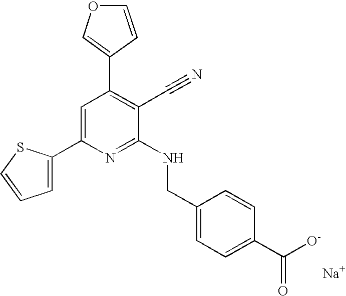 | Sodium; 4-[(3-cyano-4- furan-3-yl-6-thiophen- 2-yl-pyridin-2- ylamino)-methyl]- benzoate | | 2.20 (M2) | 402 (M − Na+) |
| 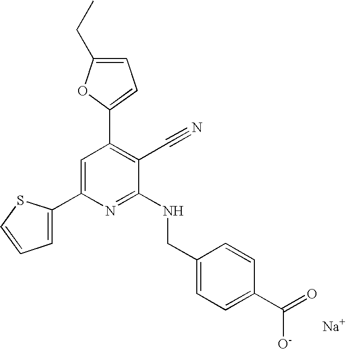 | Sodium; 4-[3-cyano-4- (5-ethyl-furan-2-yl)-6- thiophen-2-yl-pyridin- 2-yloxymethyl]- benzoate | | | |
| 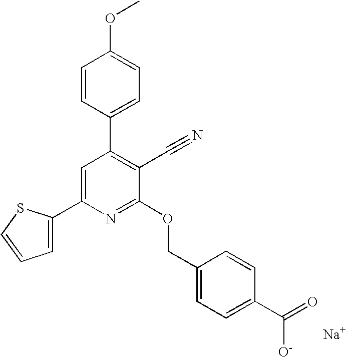 | Sodium; 4-[3-cyano-4- (4-methoxy-phenyl)-6- thiophen-2-yl-pyridin- 2-yloxymethyl]- benzoate | | | |
| 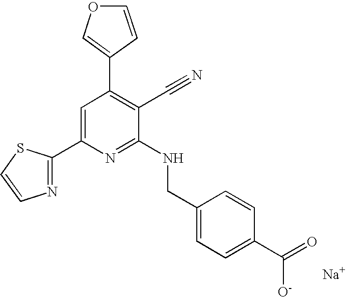 | Sodium; 4-(3-cyano-4- furan-3-yl-6-thiazol-2- yl-pyridin-2- yloxymethyl)-benzoate | | | |
| 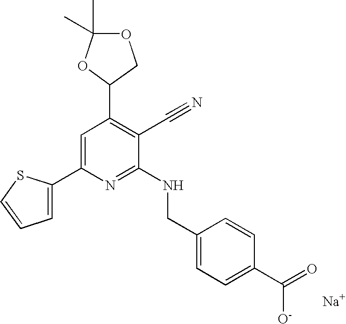 | Sodium; 4-[3-cyano-4- (2,2-dimethyl- [1,3]dioxolan-4-yl)-6- thiophen-2-yl-pyridin- 2-yloxymethyl]- benzoate | | | |
| 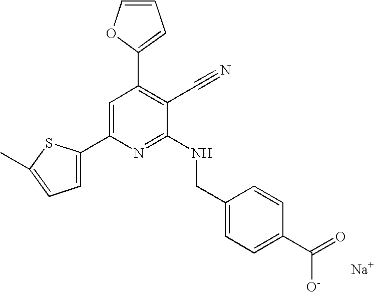 | Sodium; 4-[3-cyano-4- furan-2-yl-6-(5-methyl- thiophen-2-yl)-pyridin- 2-yloxymethyl]- benzoate | | | |
| 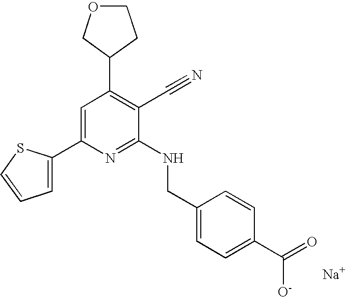 | Sodium; 4-[3-cyano-4- (tetrahydro-furan-3-yl)- 6-thiophen-2-yl- pyridin-2- yloxymethyl]-benzoate | | | |
| 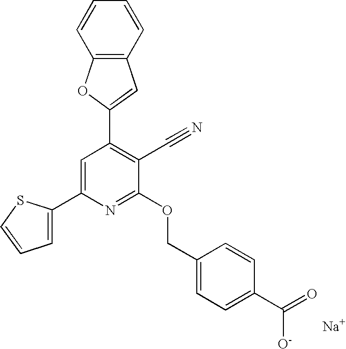 | Sodium; 4-(4- benzofuran-2-yl-3- cyano-6-thiophen-2-yl- pyridin-2- yloxymethyl)-benzoate | | | |
| 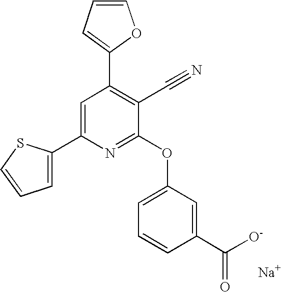 | Sodium; 3-(3-cyano-4- furan-2-yl-6-thiophen- 2-yl-pyridin-2-yloxy)- benzoate | | | |
| 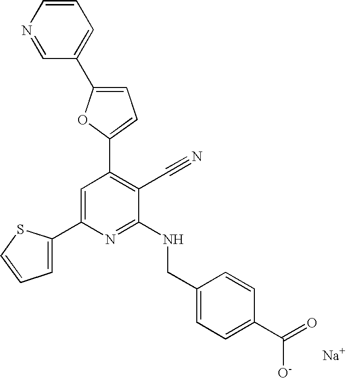 | Sodium; 4-[3-cyano-4- (5-pyridin-3-yl-furan-2- yl)-6-thiophen-2-yl- pyridin-2- yloxymethyl]-benzoate | | | |
| 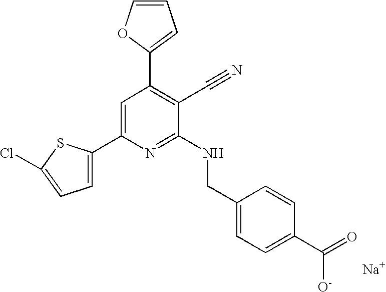 | Sodium; 4-[6-(5-chloro- thiophen-2-yl)-3-cyano- 4-furan-2-yl-pyridin-2- yloxymethyl]-benzoate | | | |
| 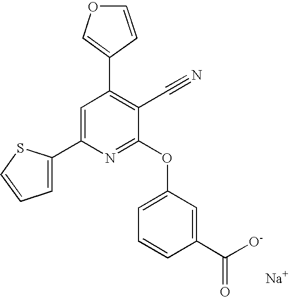 | Sodium; 3-(3-cyano-4- furan-3-yl-6-thiophen- 2-yl-pyridin-2-yloxy)- benzoate | | | |
| 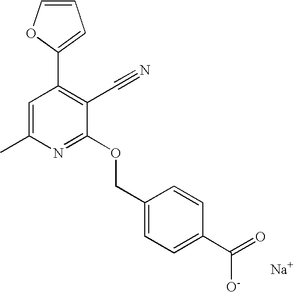 | Sodium; 4-(3-cyano-4- furan-2-yl-6-methyl- pyridin-2- yloxymethyl)-benzoate | | | |
| 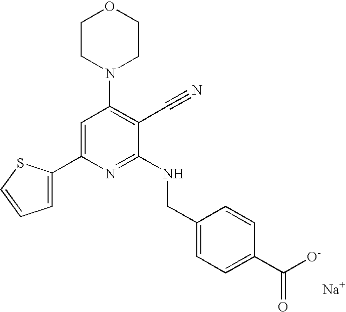 | Sodium; 4-(3-cyano-4- morpholin-4-yl-6- thiophen-2-yl-pyridin- 2-yloxymethyl)- benzoate | y | 1.99 (M2) | 422 (M − Na+) |
| 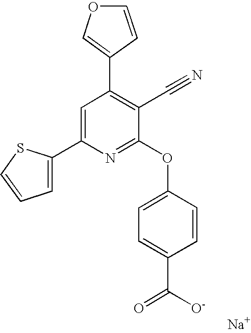 | Sodium; 4-(3-cyano-4- furan-3-yl-6-thiophen- 2-yl-pyridin-2-yloxy)- benzoate | | | |
| 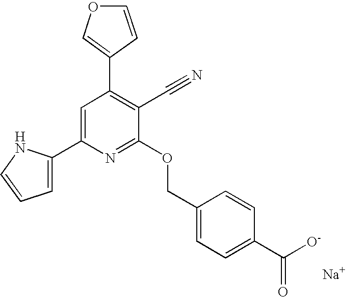 | Sodium; 4-[3-cyano-4- furan-3-yl-6-(1H- pyrrol-2-yl)-pyridin-2- yloxymethyl]-benzoate | | 1.97 (M2) | |
| 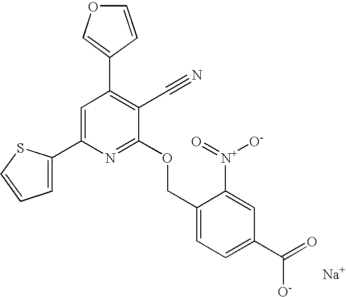 | Sodium; 4-(3-cyano-4- furan-3-yl-6-thiophen- 2-yl-pyridin-2- yloxymethyl)-3-nitro- benzoate | | | |
| 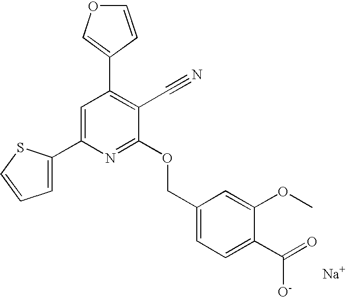 | Sodium; 4-(3-cyano-4- furan-3-yl-6-thiophen- 2-yl-pyridin-2- yloxymethyl)-2- methoxy-benzoate | | | |
| 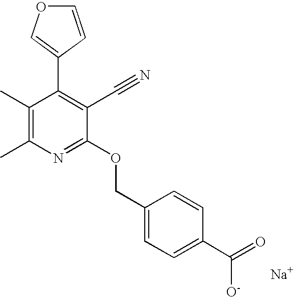 | Sodium; 4-(3-cyano-4- furan-3-yl-5,6- dimethyl-pyridin-2- yloxymethyl)- benzoate5 | | | |
| 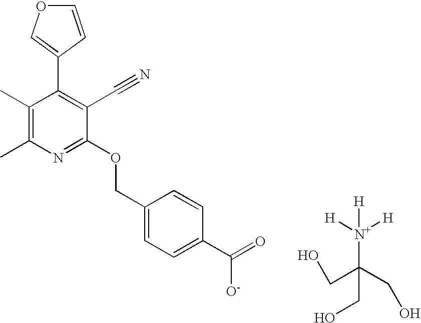 | 4-(3-Cyano-4-furan-3- yl-5,6-dimethyl- pyridin-2- yloxymethyl)- benzoate2-hydroxy-1,1- bis-hydroxymethyl- ethyl-ammonium | | | |
| 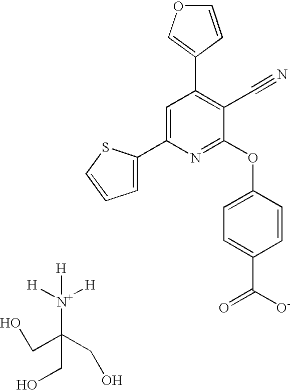 | 4-(3-Cyano-4-furan-3- yl-6-thiophen-2-yl- pyridin-2-yloxy)- benzoate2-hydroxy-1,1- bis-hydroxymethyl- ethyl-ammonium | | | |
| 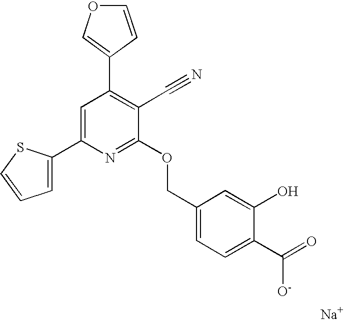 | Sodium; 4-(3-cyano-4-furan-3-yl-6-thiophen- 2-yl-pyridin-2- yloxymethyl)-2- hydroxy-benzoate | | | |
| 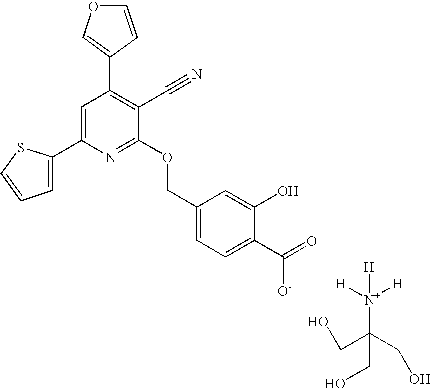 | 4-(3-Cyano-4-furan-3- yl-6-thiophen-2-yl- pyridin-2- yloxymethyl)-2- hydroxy-benzoate2- hydroxy-1,1-bis- hydroxymethyl-ethyl- ammonium | | 2.11 (M2) | (M + H+) |
| 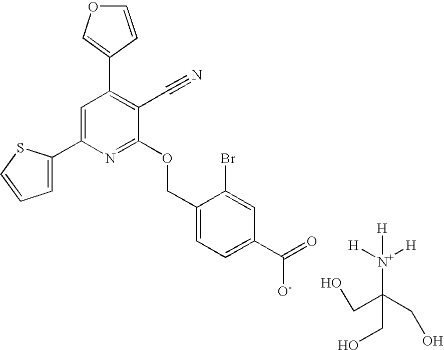 | 3-Bromo-4-(3-cyano-4- furan-3-yl-6-thiophen- 2-yl-pyridin-2- yloxymethyl)- benzoate2-hydroxy-1,1- bis-hydroxymethyl- ethyl-ammonium | | | |
| 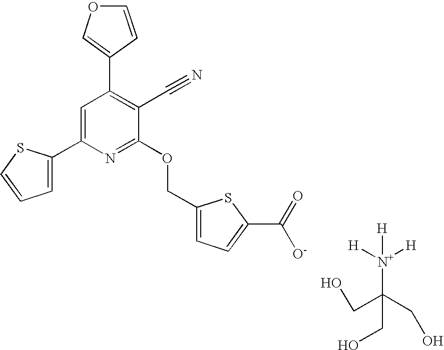 | 5-(3-Cyano-4-furan-3- yl-6-thiophene-2-yl- pyridin-2- yloxymethyl)- thiophen-2- carboxylate2-hydroxy- 1,1-bis-hydroxymethyl- ethyl-ammonium | | 2.02 (M2) | 431(m + Na+) |
| 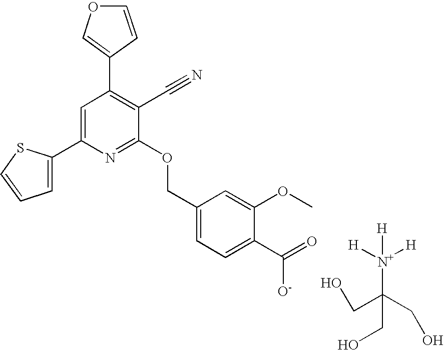 | 4-(3-Cyano-4-furan-3- yl-6-thiophen-2-yl- pyridin-2- yloxymethyl)-2- methoxy-benzoate2- hydroxy-1,1-bis- hydroxymethyl-ethyl- ammonium | | 2.06 (M2) | (M + H+) |
| 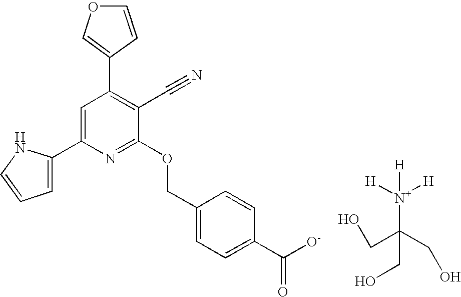 | 4-[3-Cyano-4-furan-3- yl-6-(1H-pyrrol-2-yl)- pyridin-2- yloxymethyl]- benzoate2-hydroxy-1,1- bis-hydroxymethyl- ethyl-ammonium | | 2.06 (M2) | (M + H+) |
| 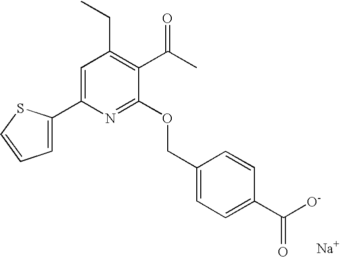 | Sodium; 4-(3-acetyl-4- ethyl-6-thiophen-2-yl- pyridin-2- yloxymethyl)-benzoate | | | |
| 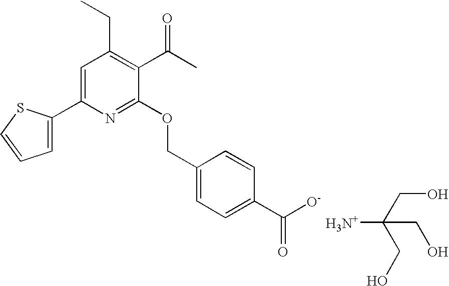 | 4-(3-Acetyl-4-ethyl-6- thiophen-2-yl-pyridin- 2-yloxymethyl)- benzoate2-hydroxy-1,1- bis-hydroxymethyl- ethyl-ammonium | | | |
| 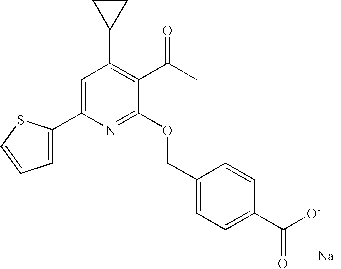 | Sodium; 4-(3-acetyl-4- cyclopropyl-6- thiophen-2-yl-pyridin- 2-yloxymethyl)- benzoate | | | |
| 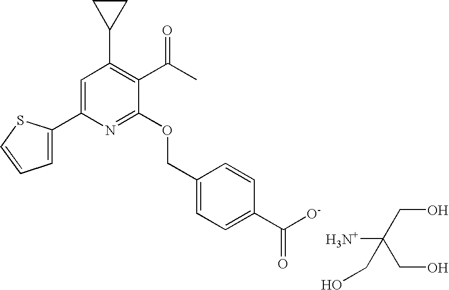 | 4-(3-Acetyl-4- cyclopropyl-6- thiophen-2-yl-pyridin- 2-yloxymethyl)- benzoate2-hydroxy-1,1- bis-hydroxymethyl- ethyl-ammonium | | | |
| 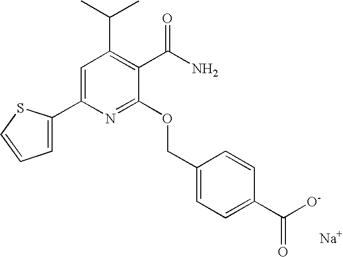 | Sodium; 4-(3- carbamoyl-4-isopropyl- 6-thiophen-2-yl- pyridin-2- yloxymethyl)-benzoate | | | |
| 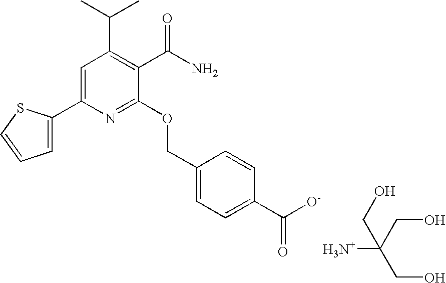 | 4-(3-Carbamoyl-4- isopropyl-6-thiophen-2- yl-pyridin-2- yloxymethyl)- benzoate2-hydroxy-1,1- bis-hydroxymethyl- ethyl-ammonium | | | |
| 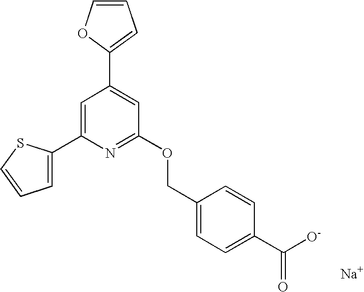 | Sodium; 4-(4-furan-2- yl-6-thiophen-2-yl- pyridin-2- yloxymethyl)-benzoate | | 2.08 (M2) | 378 (M + H+) |
| 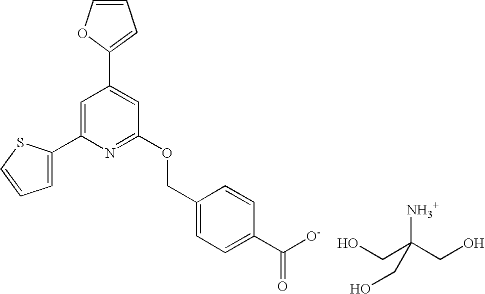 | 4-(4-Furan-2-yl-6- thiophen-2-yl-pyridin- 2-yloxymethyl)- benzoate2-hydroxy-1,1- bis-hydroxymethyl- ethyl-ammonium | | 2.08 (M2) | 378 (M + H+) |
| 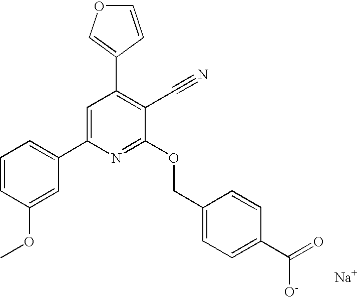 | Sodium; 4-[3-cyano-4- furan-3-yl-6-(3- methoxy-phenyl)- pyridin-2- yloxymethyl]-benzoate | | | |
| 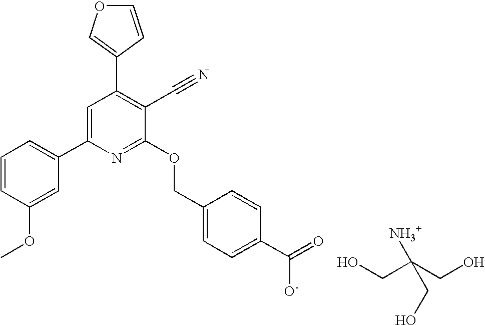 | 4-[3-Cyano-4-furan-3- yl-6-(3-methoxy- phenyl)-pyridin-2- yloxymethyl]- benzoate2-hydroxy-1,1- bis-hydroxymethyl- ethyl-ammonium | | 2.09 (M2) | 427 (M + H+) |
| 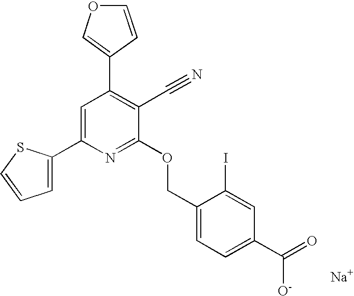 | Sodium; 4-(3-cyano-4- furan-3-yl-6-thiophen- 2-yl-pyridin-2- yloxymethyl)-3-iodo- benzoate | | 2.20 (M2) | 529 (M + H+) |
| 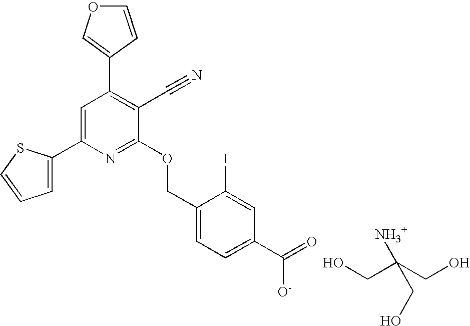 | 4-(3-Cyano-4-furan-3- yl-6-thiophen-2-yl- pyridin-2- yloxymethyl)-3-iodo- benzoate2-hydroxy-1,1- bis-hydroxymethyl- ethyl-ammonium | | 2.21 (M2) | 529 (M + H+) |
| 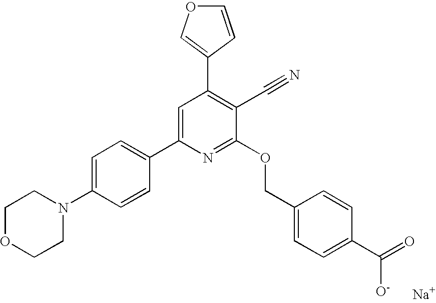 | Sodium; 4-[3-cyano-4- furan-3-yl-6-(4- morpholin-4-yl- phenyl)-pyridin-2- yloxymethyl]-benzoate | | 2.08 (M2) | 504 (M + Na+) |
| 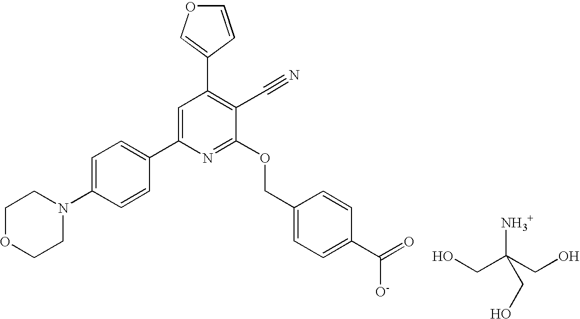 | 4-[3-Cyano-4-furan-3- yl-6-(4-morpholin-4-yl- phenyl)-pyridin-2- yloxymethyl]- benzoate2-hydroxy-1,1- bis-hydroxymethyl- ethyl-ammonium | | 2.08 (M2) | 482 (M + H+) |
| 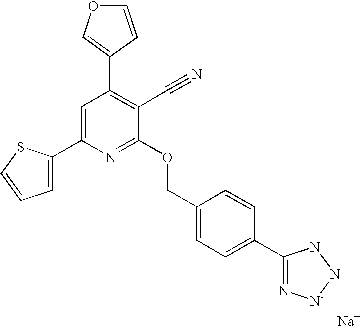 | 4-Furan-3-yl-2-[4-(2H- tetrazol-5-yl)- benzyloxy]-6-thiophen- 2-yl-nicotnonitrile; sodium salt | | | |
| 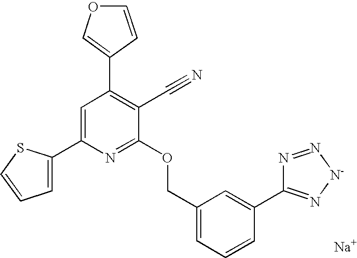 | 4-Furan-3-yl-2-[3-(2H- tetrazol-5-yl)- benzyloxy]-6-thiophen- 2-yl-nicotinonitrile; sodium salt | | 2.12 (M2) | |
| 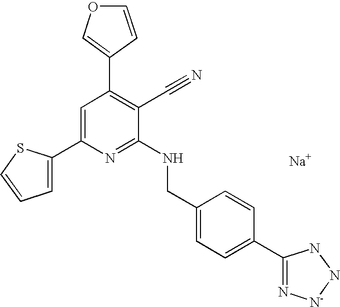 | 4-Furan-3-yl-2-[4-(2H- tetrazol-5-yl)- benzylamino]-6- thiophen-2-yl- nicotinonitrile | | | |

Further compounds and salts

| | | | | | HPLC retention |
|---|---|---|---|---|---|
| | | | ¹H | | time for salt |
| STRUCTURE | MW | Name | NMR | parent Lc/ms | (1c method) |
| 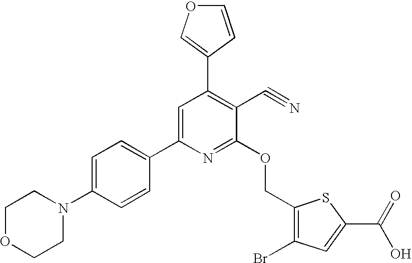 | 566.43 | 4-Bromo-5-[3-cyano-4-furan- 3-yl-6-(4-morpholin-4-yl- phenyl)-pyridin-2- yloxymethyl]-thiophene- 2-carboxylic acid | Y | | 2.9 (M2) |
| 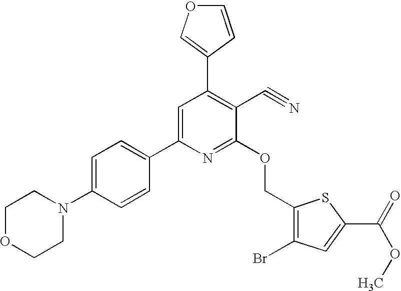 | 580.46 | 4-Bromo-5-[3-cyano-4-furan- 3-yl-6-(4-morpholin-4-yl- phenyl)-pyridin-2- yloxymethyl]-thiophene-2- carboxylic acid methyl ester | Y | 604 (M + Na+) | 1.90 (M2) |
| 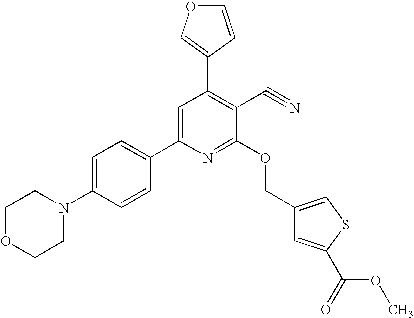 | 501.57 | 4-[3-Cyano-4-furan-3-yl-6-(4- morpholin-4-yl-phenyl)- pyridin-2-yloxymethyl]-thiophene-2-carboxylic acid methyl ester | Y | 524 | 1.7 (M2) |
| 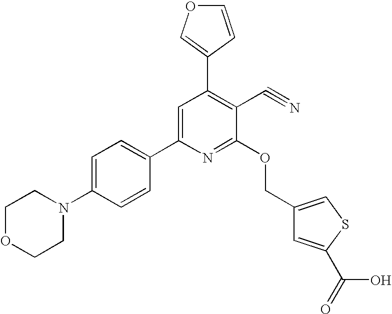 | 487.54 | 4-[3-Cyano-4-furan-3-yl-6-(4- morpholin-4-yl-phenyl)- pyridin-2-yloxymethyl]- thiophene-2-carboxylic acid | Y | 549 | |
| 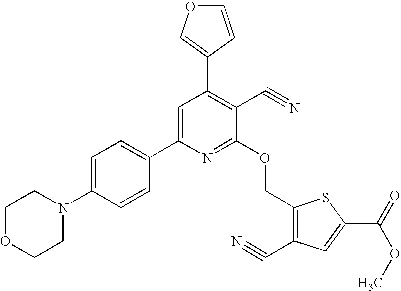 | 526.58 | 4-Cyano-5-[3-cyano-4-furan- 3-yl-6-(4-morpholin-4-yl- phenyl)-pyridin-2- yloxymethyl]-thiophene-2- carboxylic acid methyl ester | Y | | |
| 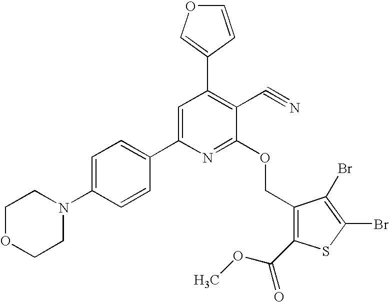 | 659.36 | 4,5-Dibromo-3-[3-cyano-4- furan-3-yl-6-(4-morpholin-4- yl-phenyl)-pyridin-2- yloxymethyl]-thiophene-2- carboxylic acid methyl ester | Y | | |
| 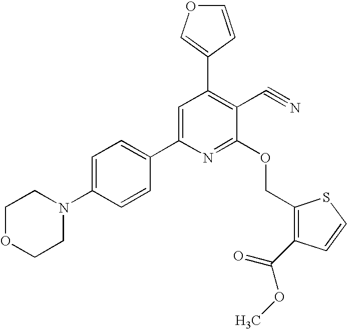 | 501.57 | 2-[3-Cyano-4-furan-3-yl-6-(4- morpholin-4-yl-phenyl)- pyridin-2-yloxymethyl]- thiophene-3-carboxylic acid methyl ester | Y | 529 (M + Na+) | 17.5 (M3) |
| 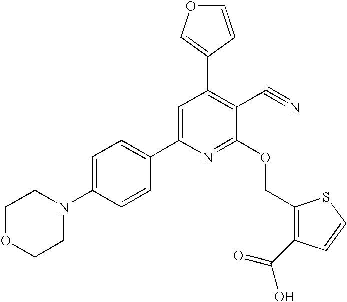 | 487.54 | 3-[3-Cyano-4-furan-3-yl-6- (4-morpholin-4-yl-phenyl)- pyridin-2-yloxymethyl]- thiophene-2-carboxylic acid | Y | 486 (M − H+) | 17.8 (M3) |
| 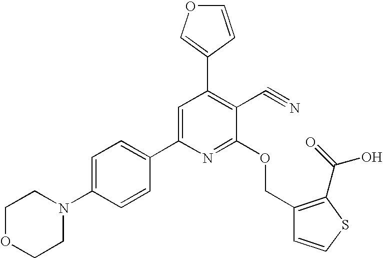 | 487.54 | 3-[3-Cyano-4-furan-3-yl-6- (4-morpholin-4-yl-phenyl)- pyridin-2-yloxymethyl]- thiophene-2-carboxylic acid | Y | 502.1 (M + Na+) | |
| 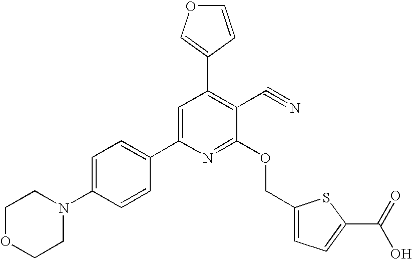 | 487.54 | 4-Furan-3-yl-2-(5-hydroxy- thiophen-2-ylmethoxy)-6- (4-morpholin-4-yl-phenyl)- nicotinonitrile | Y | 524 (M + K+) | |
| 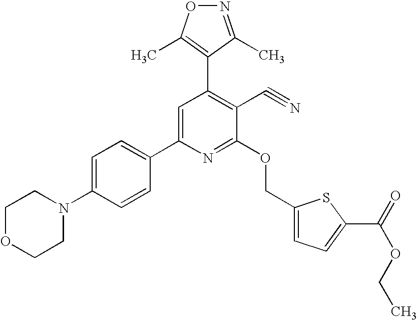 | 544.63 | 5-[3-Cyano-4-(3,5-dimethyl- isoxazol-4-yl)-6-(4-morpholin- 4-yl-phenyl)-pyridin-2- yloxymethyl]-thiophene-2- carboxylic acid ethyl ester | Y | 539 | |
| 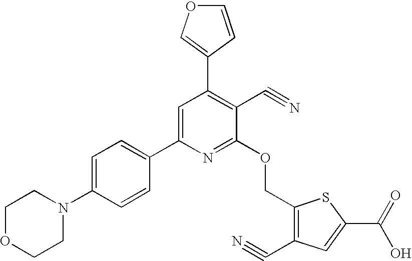 | 512.55 | 4-Cyano-5-[3-cyano-4-furan- 3-yl-6-(4-morpholin-4-yl- phenyl)-pyridin-2- yloxymethyl]-thiophene- 2-carboxylic acid | Y | 513 (M + H+) | |
| 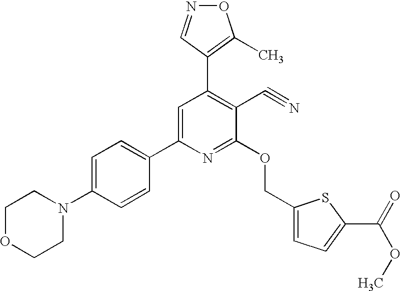 | 516.58 | 5-[3-Cyano-4-(5-methyl- isoxazol-4-yl)-6-(4- morpholin-4-yl-phenyl)- pyridin-2-yloxymethyl]- thiophene-2-carboxylic acid methyl ester | Y | 501 | |
| 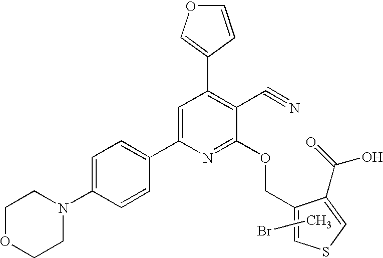 | 582.48 | 2-Bromo-4-[3-cyano-4-furan- 3-yl-6-(4-morpholin-4-yl- phenyl)-pyridin-2- yloxymethyl]-thiophene-3- carboxylic acid | Y | | |
| 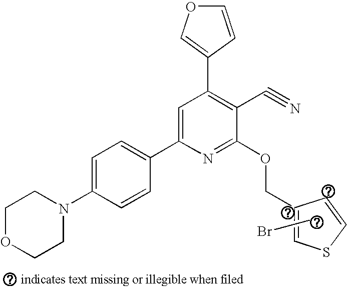 | 677.42 | 3,5-Dibromo-4-[3-cyano-4- furan-3-yl-6-(4-morpholin- 4-yl-phenyl)-pyridin-2- yloxymethyl]-thiophene-2- carboxylic acid | Y | | |
| 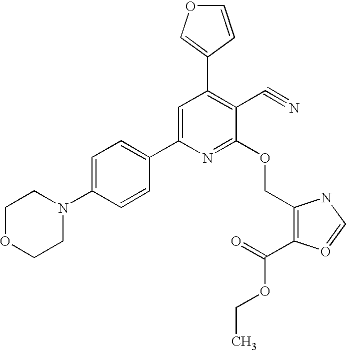 | 500.52 | 4-[3-Cyano-4-furan-3-yl-6- (4-morpholin-4-yl-phenyl)- pyridin-2-yloxymethyl]- oxazole-5-carboxylic acid ethyl ester | Y | 499 (M − H+) | 15.2 (M3) |
| 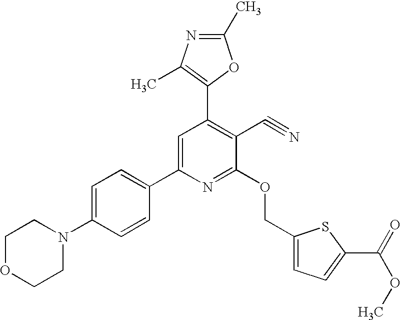 | 530.61 | 5-[3-Cyano-4-(2,4-dimethyl- oxazol-5-yl)-6-(4-morpholin- 4-yl-phenyl)-pyridin-2- yloxymethyl]-thiophene-2- carboxylic acid methyl ester | Y | 551 (M + Na+) | 18.62 (M3) |
| 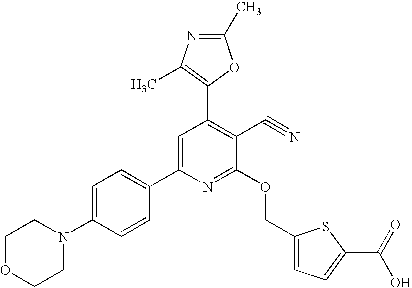 | 516.58 | 5-[3-Cyano-4-(2,4-dimethyl- oxazol-5-yl)-6-(4-morpholin- 4-yl-phenyl)-pyridin-2- yloxymethyl]-thiophene-2- carboxylic acid | Y | 515 (M − H+) | |
| 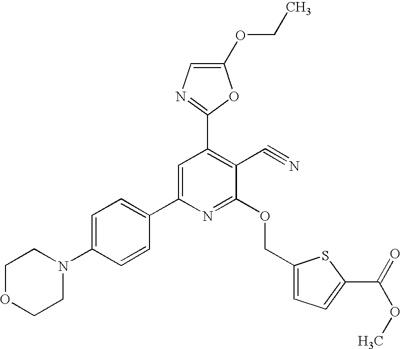 | 546.61 | 5-[3-Cyano-4-(5-ethoxy- oxazol-2-yl)-6-(4-morpholin- 4-yl-phenyl)-pyridin-2- yloxymethyl]-thiophene-2- carboxylic acid methyl ester | Y | | 13.95 (M3) |
| 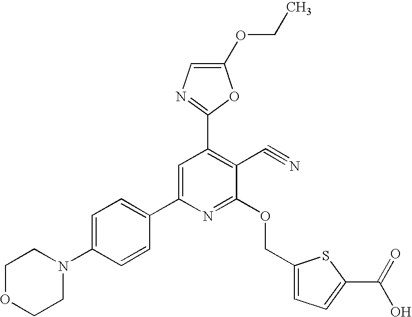 | 532.58 | 5-[3-Cyano-4-(5-ethoxy- oxazol-2-yl)-6-(4-morpholin- 4-yl-phenyl)-pyridin-2- yloxymethyl]-thiophene-2- carboxylic acid | Y | 553 (M + H+) | 17.18 (M3) |
| 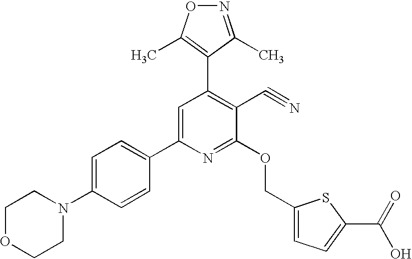 | 516.58 | 5-[3-Cyano-4-(3,5-dimethyl- isoxazol-4-yl)-6-(4-morpholin- 4-yl-phenyl)-pyridin-2- carboxylic acid | Y | 517 (M + H+) | 13.72 (M3) |
| 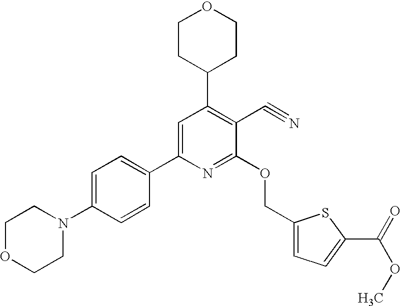 | 519.62 | 5-[3-Cyano-6-(4-morpholin-4- yl-phenyl)-4-(tetrahydro- pyran-4-yl)-pyridin-2- yloxymethyl]-thiophene-2- carboxylic acid methyl ester | Y | 542 (M + Na+) | 15.6 (M3) |
| 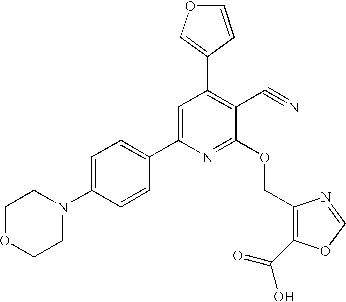 | 472.46 | 4-[3-Cyano-4-furan-3-yl-6-(4- morpholin-4-yl-phenyl)- pyridin-2-yloxymethyl]- oxazole-5-carboxylic acid | Y | | |
| 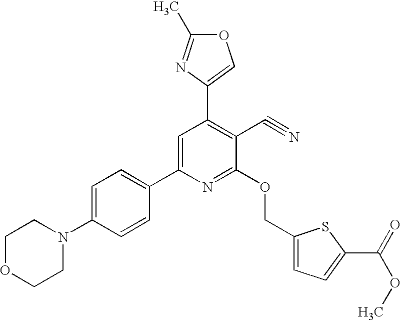 | 516.58 | 5-[3-Cyano-4-(2-methyl- oxazol-4-yl)-6-(4-morpholin- 4-yl-phenyl)-pyridin-2- yloxymethyl]-thiophene-2- carboxylic acid methyl ester | Y | | 13.06 (M3) |
| 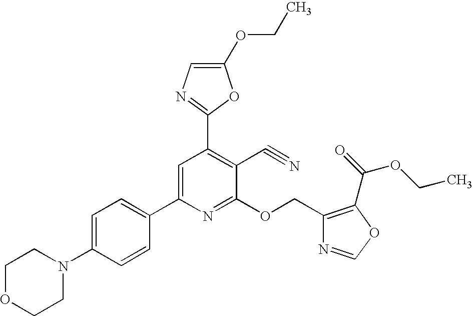 | 545.56 | 4-[3-Cyano-4-(5-methyl- oxazol-2-yl)-6-(4-morpholin- 4-yl-phenyl)-pyridin-2- yloxymethyl]-oxazole-5- carboxylic acid ethyl ester | Y | 568 (M + Na +) | |
| 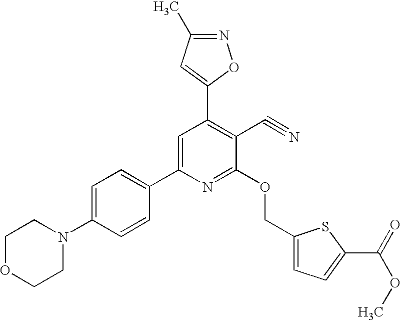 | 516.58 | 5-[3-Cyano-4-(3-methyl- isoxazol-5-yl)-6-(4-morpholin- 4-yl-phenyl)-pyridin-2- yloxymethyl]-thiophene-2- carboxylic acid methyl ester | Y | 539 (M + Na +) | |
| 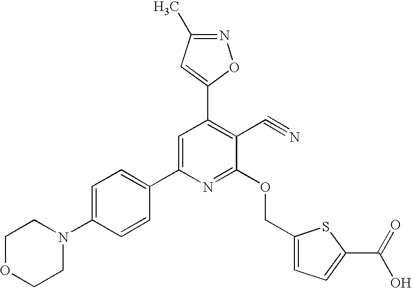 | 502.55 | 5-[3-Cyano-4-(3-methyl- isoxazol-5-yl)-6-(4-morpholin- 4-yl-phenyl)-pyridin-2- yloxymethyl]-thiophene-2- carboxylic acid | Y | 501 (M − H +) | |
| 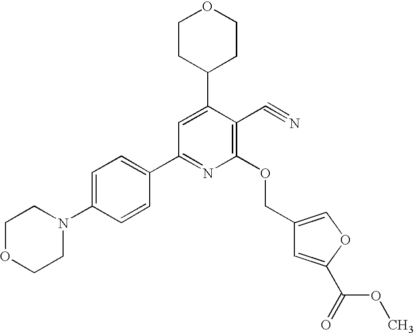 | 519.62 | 4-[3-Cyano-6-(4-morpholin-4- yl-phenyl)-4-(tetrahydro- pyran-4-yl)-pyridin-2- yloxymethyl]-thiophene-2- carboxylic acid methyl ester | Y | 542 (M + Na +) | |
| 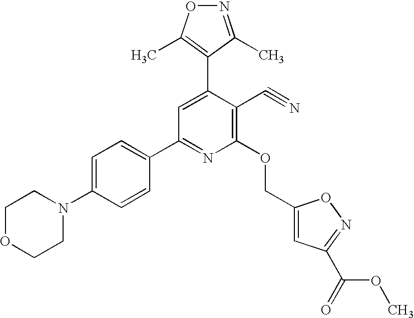 | 529.56 | 5-[3-Cyano-4-(3,5-dimethyl- isoxazol-4-yl)-6-(4-morpholin- 4-yl-phenyl)-pyridin-2- yloxymethyl]-isoxazole-3- carboxylic acid ethyl ester | Y | 552 (M + Na +) | |
| 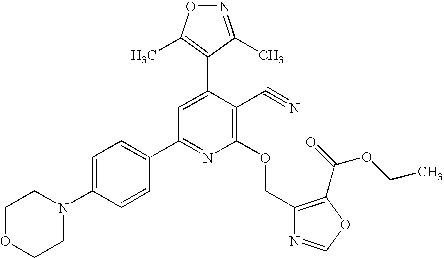 | 529.56 | 4-[3-Cyano-4-(3,5-dimethyl- isoxazol-4-yl)-6-(4-morpholin- 4-yl-phenyl)-pyridin-2- yloxymethyl]-oxazole-5- carboxylic acid ethyl ester | Y | 552 (M + Na +) | |
| 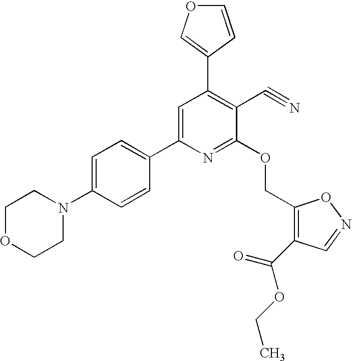 | 500.52 | 5-[3-Cyano-4-furan-3-yl-6- (4-morpholin-4-yl-phenyl)- pyridin-2-yloxymethyl]- isoxazole-4-carboxylic acid ethyl ester | Y | 499 (M − H +) | |
| 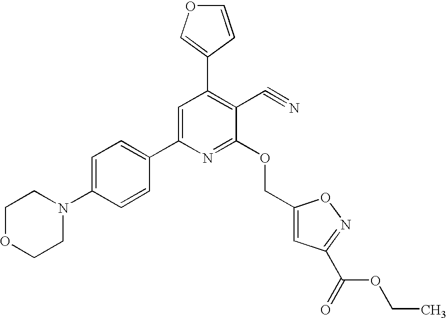 | 500.52 | 5-[3-Cyano-4-furan-3-yl-6- (4-morpholin-4-yl-phenyl)- pyridin-2-yloxymethyl]- isoxazole-3-carboxylic acid ethyl ester | Y | 523 (M + Na +) | |
| 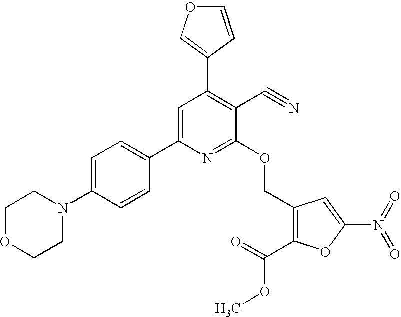 | 546.56 | 3-[3-Cyano-4-furan-3-yl-6- (4-morpholin-4-yl-phenyl)- pyridin-2-yloxymethyl]-5- nitro-thiophene-2-carboxylic acid methyl ester | Y | 569 (M + Na +) | |
| 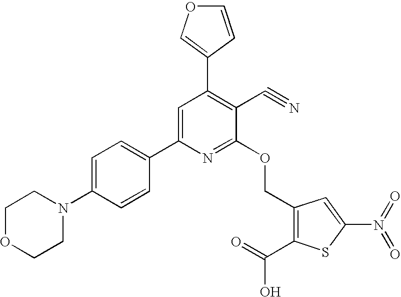 | 532.54 | 3-[3-Cyano-4-furan-3-yl-6- (4-morpholin-4-yl-phenyl)- pyridin-2-yloxymethyl]-5- nitro-thiophene-2-carboxylic acid | Y | 531 (M − H +) | |
| 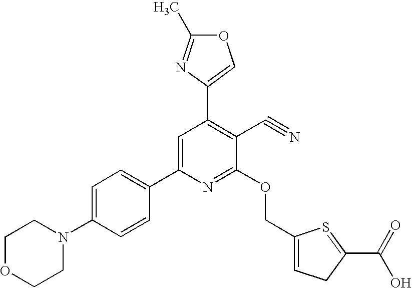 | 502.55 | 5-[3-Cyano-4-(2-methyl- oxazol-4-yl)-6-(4-morpholin- 4-yl-phenyl)-pyridin-2- yloxymethyl]-thiophene-2- carboxylic acid | Y | | 17.9 (M3) |
| 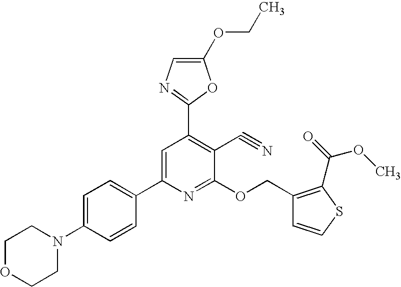 | 546.61 | 3-[3-Cyano-4-(5-ethoxy- oxazol-2-yl)-6-(4-morpholin- 4-yl-phenyl)-pyridin-2- yloxymethyl]-thiophene-2- carboxylic acid methyl ester | Y | 569 (M + Na +) | |
| 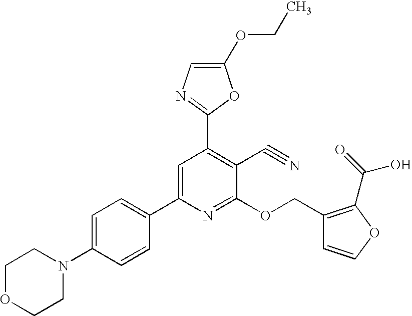 | 517.5 | 4-[3-Cyano-4-(5-ethoxy- oxazol-2-yl)-6-(4-morpholin- 4-yl-phenyl)-pyridin-2- yloxymethyl]-oxazole-5- carboxylic acid | Y | 516 (M − H +) | |
| 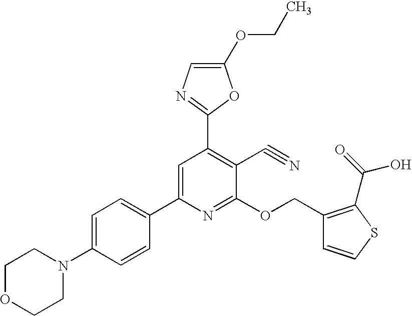 | 532.58 | 3-[3-Cyano-4-(5-ethoxy- oxazol-2-yl)-6-(4-morpholin- 4-yl-phenyl)-pyridin-2- yloxymethyl]-thiophene-2- carboxylic acid | Y | | 14.7 (M3) |
| 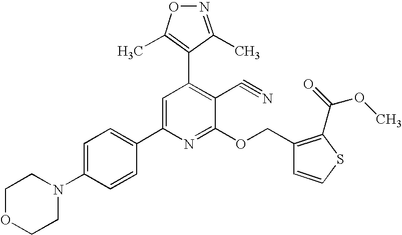 | 530.61 | 3-[3-Cyano-4-(3,5-dimethyl- isoxazol-4-yl)-6-(4-morpholin- 4-yl-phenyl)-pyridin-2- yloxymethyl]-thiophene-2- carboxylic acid methyl ester | Y | 552 (M + H +) | 19.32 (M3) |
| 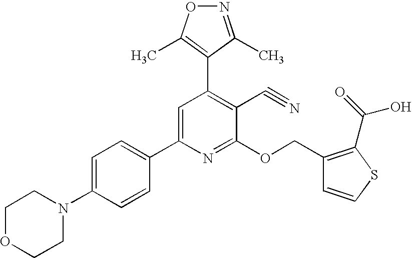 | 516.58 | 3-[3-Cyano-4-(3,5-dimethyl- isoxazol-4-yl)-6-(4-morpholin- 4-yl-phenyl)-pyridin-2- yloxymethyl]-thiophene-2- carboxylic acid | Y | | 13.8 (M3) |
| 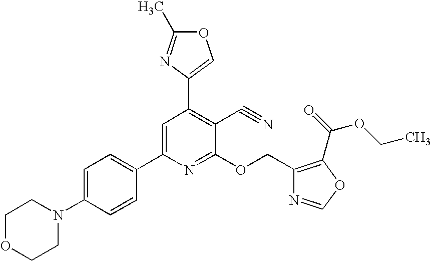 | 515.53 | 4-[3-Cyano-4-(2-methyl- oxazol-4-yl)-6-(4-morpholin- 4-yl-phenyl)-pyridin-2- yloxymethyl]-oxazole-5- carboxylic acid ethyl ester | Y | 538 (M + Na +) | 14.8 (M3) |
| 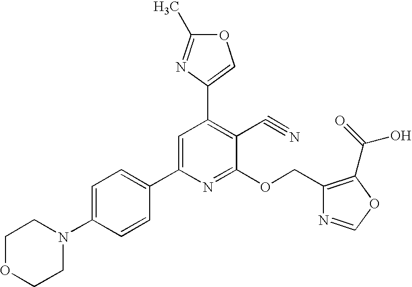 | 487.48 | 4-[3-Cyano-4-(2-methyl- oxazol-4-yl)-6-(4-morpholin- 4-yl-phenyl)-pyridin-2- yloxymethyl]-oxazole-5- carboxylic acid | Y | | 12.08 (M3) |
| 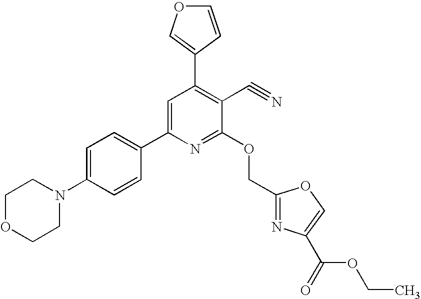 | 500.52 | 2-[3-Cyano-4-furan-3-yl-6- (4-morpholin-4-yl-phenyl)- pyridin-2-yloxymethyl]- oxazole-4-carboxylic acid | Y | 523 (M + Na +) | 14.8 (M3) |
| 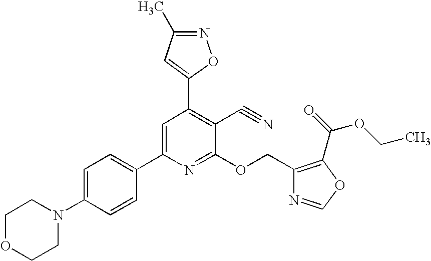 | 515.53 | 4-[3-Cyano-4-(2-methyl- oxazol-4-yl)-6-(4-morpholin- 4-yl-phenyl)-pyridin-2- yloxymethyl]-oxazole-5- carboxylic acid ethyl ester | Y | 538 (M + Na +) | 15.0 (M3) |
| 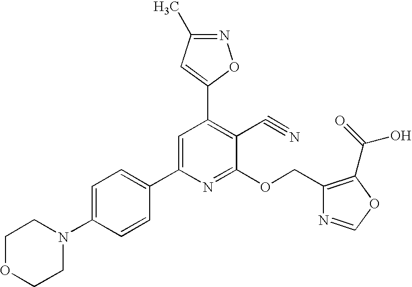 | 487.48 | 4-[3-Cyano-4-(3-methyl- isoxazol-5-yl)-6-(4- morpholin-4-yl-phenyl)- pyridin-2-yloxymethyl]- oxazole-5-carboxylic acid | Y | 486 (M − H +) | 12.5 (M3) |
| 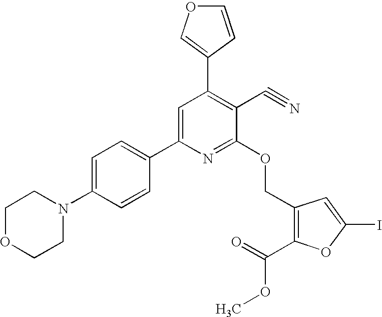 | 627.46 | 3-[3-Cyano-4-furan-3-yl-6- (4-morpholin-4-yl-phenyl)- pyridin-2-yloxymethyl]-5- iodo-thiophene-2-carboxylic acid methyl ester | Y | 650 (M + Na +) | 17.0 (M3) |
| 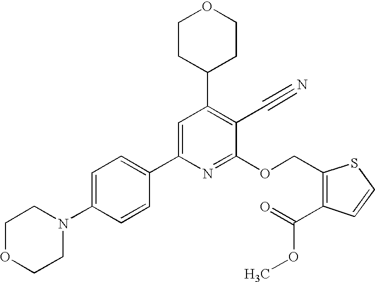 | 519.62 | 2-[3-Cyano-6-(4-morpholin-4- yl-phenyl)-4-(tetrahydro- pyran-4-yl)-pyridin-2- yloxymethyl]-thiophene-3- carboxylic acid methyl ester | Y | 542 (M + Na+) | 16.03 (M3) |
| 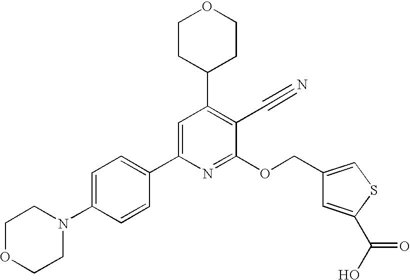 | 505.6 | 4-[3-Cyano-6-(4-morpholin-4- yl-phenyl)-4-(tetrahydropyran- 4-yl)-pyridin-2- yloxymethyl]-thiophene-2- carboxylic acid | Y | 504 (M − H+) | 13.4 (M3) |
| 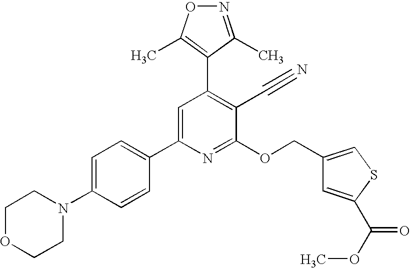 | 530.61 | 4-[3-Cyano-4-(3,5-dimethyl- isoxazol-4-yl)-6-(4-morpholin- 4-yl-phenyl)-pyridin-2- yloxymethyl]-thiophene-2- carboxylic acid methyl ester | Y | | 1.45 (M2) |
| 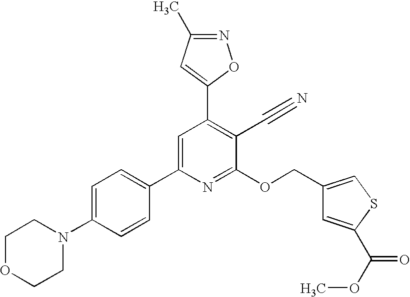 | 516.58 | 4-[3-Cyano-4-(3-methyl- isoxazol-5-yl)-6-(4-morpholin- 4-yl-phenyl)-pyridin-2- yloxymethyl]-thiophene-2- carboxylic acid methyl ester | Y | 553 (M + K+) | 15.54 (M3) |
| 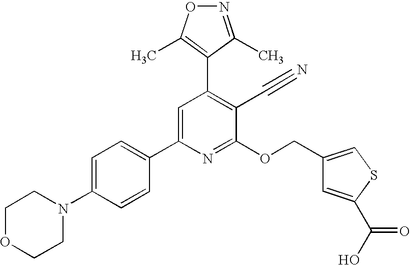 | 516.58 | 4-[3-Cyano-4-(3,5-dimethyl- isoxazol-4-yl)-6-(4-morpholin- 4-yl-phenyl)-pyridin-2- yloxymethyl]-thiophene-2- carboxylic acid | Y | 515 (M − H+) | 13.51 (M3) |
| 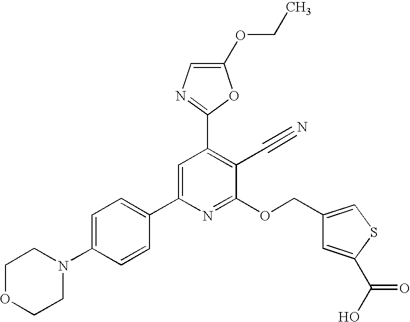 | 532.58 | 2-[2-(5-Carboxy-thiophen-3- ylmethoxy)-3-cyano-6-(4- morpholin-4-yl-phenyl)- pyridin-4-yl]-oxazole-5- carboxylic acid ethyl ester | Y | 531 (M − H+) | 14.72 (M3) |
| 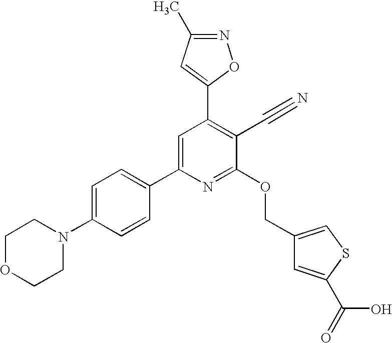 | 502.55 | 4-[3-Cyano-4-(3-methyl- isoxazol-5-yl)-6-(4-morpholin- 4-yl-phenyl)-pyridin-2- yloxymethyl]-thiophene-2- carboxylic acid | Y | 501 (M − H+) | 14.77 (M3) |
| 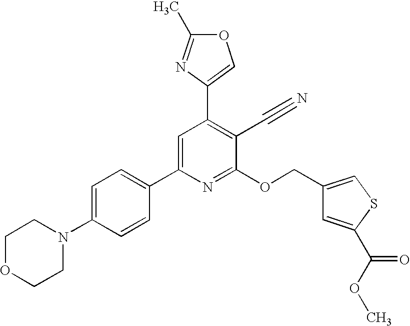 | 516.58 | 4-[3-Cyano-4-(2-methyl- oxazol-4-yl)-6-(4-morpholin- 4-yl-phenyl)-pyridin-2- yloxymethyl]-thiophene-2- carboxylic acid methyl ester | Y | 539 (M + Na+) | 16.3 (M3) |
| 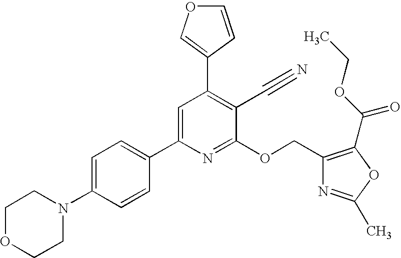 | 514.54 | 4-[3-Cyano-4-furan-3-yl-6- (4-morpholin-4-yl-phenyl)- pyridin-2-yloxymethyl]-2- methyl-oxazole-5-carboxylic acid ethyl ester | Y | 537 (M + Na+) | 15.35 (M3) |
| 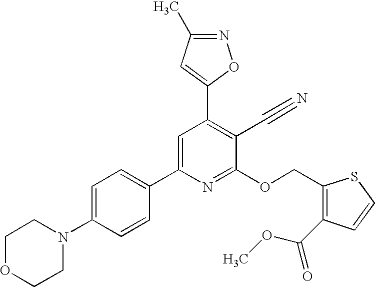 | 516.58 | 2-[3-Cyano-4-(3-methyl- isoxazol-5-yl)-6-(4-morpholin- 4-yl-phenyl)-pyridin-2- yloxymethyl]-thiophene-3- carboxylic acid methyl ester | Y | 539 (M + Na+) | 16.66 (M3) |
| 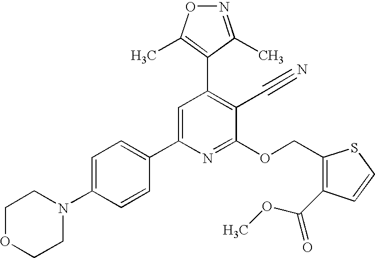 | 530.61 | 2-[3-Cyano-4-(3,5-dimethyl- isoxazol-4-yl)-6-(4-morpholin- 4-yl-phenyl)-pyridin-2- yloxymethyl]-thiophene-3- carboxylic acid methyl ester | Y | 552 (M + H+) | 15.8 (M3) |
| 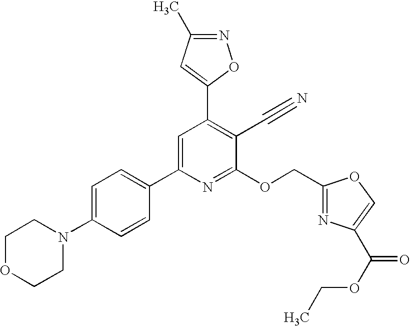 | 515.53 | 2-[3-Cyano-4-(3-methyl- isoxazol-5-yl)-6-(4-morpholin- 4-yl-phenyl)-pyridin-2- yloxymethyl]-oxazole-4- carboxylic acid ethyl ester | Y | 538 (M + Na+) | 14.85 (M3) |
| 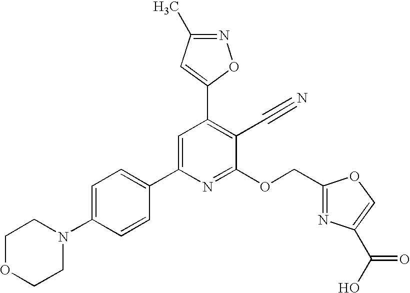 | 487.48 | 2-[3-Cyano-4-(3-methyl- isoxazol-5-yl)-6-(4-morpholin- 4-yl-phenyl)-pyridin-2- yloxymethyl]-oxazole-4- carboxylic acid | Y | 486 (M − H+) | 12.59 (M3) |
| 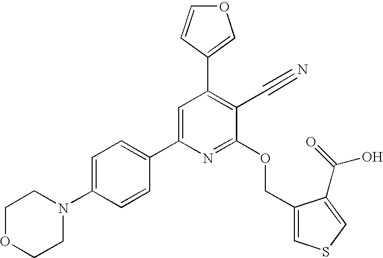 | 487.54 | 4-[3-Cyano-4-furan-3-yl-6-(4- morpholin-4-yl-phenyl)- pyridin-2-yloxymethyl]- thiophene-3-carboxylic acid | Y | 488 (M + H+) | 1.49 (M2) |
| 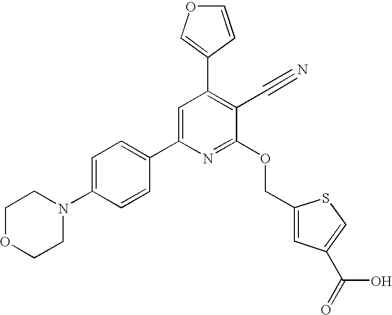 | 487.54 | 5-[3-Cyano-4-furan-3-yl-6-(4- morpholin-4-yl-phenyl)- pyridin-2-yloxymethyl]- thiophene-3-carboxylic acid | Y | | 1.83 (M1) |
| 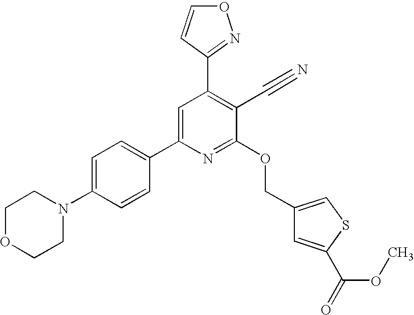 | 502.55 | 4-[3-Cyano-4-isoxazol-3-yl-6- (4-morpholin-4-yl-phenyl)- pyridin-2-yloxymethyl]- thiophene-2-carboxylic acid methyl ester | Y | 525 (M + Na+) | 15.56 (M3) |
| 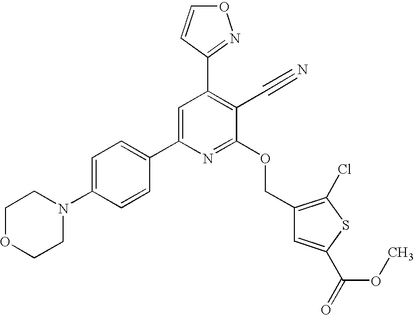 | 537 | 5-Chloro-4-[3-cyano-4- isoxazol-3-yl-6-(4-morpholin- 4-yl-phenyl)-pyridin-2- yloxymethyl]-thiophene-2- carboxylic acid methyl ester | Y | 559 (M + Na+) | 16.68 (M3) |
| 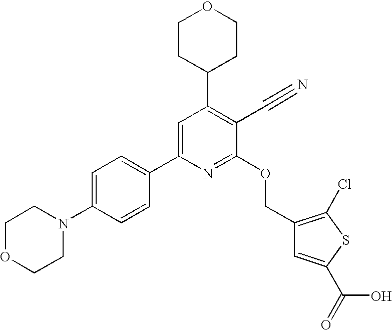 | 540.04 | 5-Chloro-4-[3-cyano-6-(4- morpholin-4-yl-phenyl)-4- (tetrahydro-pyran-4-yl)- pyridin-2-yloxymethyl]- thiophene-2-carboxylic acid | Y | 539 (M − H+) | 14.92 (M3) |
| 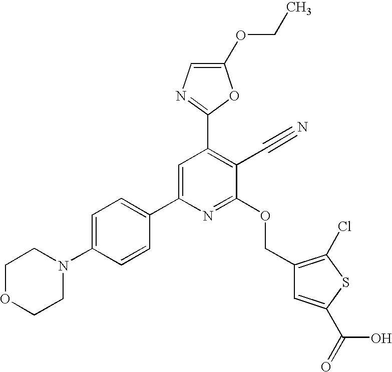 | 567.02 | 5-Chloro-4-[3-cyano-4-(5- ethoxy-oxazol-2-yl)-6-(4- morpholin-4-yl-phenyl)- pyridin-2-yloxymethyl]- thiophene-2-carboxylic acid | Y | 603 (M + Na+) | 17.5 (M3) |
| 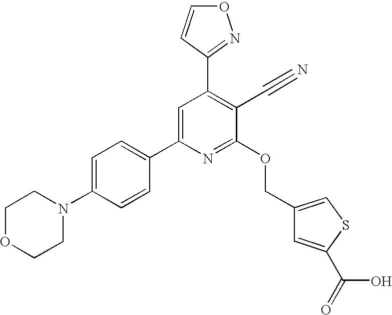 | 488.53 | 4-[3-Cyano-4-isoxazol-3-yl-6- (4-morpholin-4-yl-phenyl)- pyridin-2-yloxymethyl]- thiophene-2-carboxylic acid | Y | 487 (M − H+) | 13.71 (M3) |
| 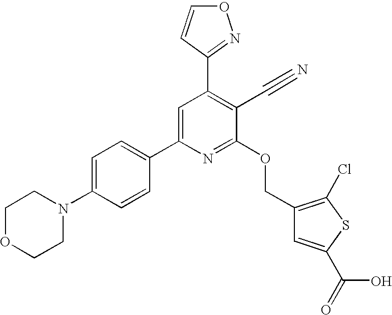 | 522.97 | 5-Chloro-4-[3-cyano-4- isoxazol-3-yl-6-(4-morpholin- 4-yl-phenyl)-pyridin-2- yloxymethyl]-thiophene-2- carboxylic acid | Y | 521 (M − H+) | 14.98 (M3) |
| 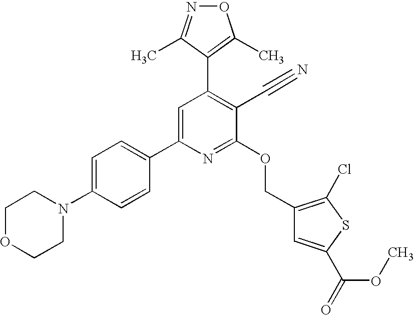 | 565.05 | 5-Chloro-4-[3-cyano-4-(3,5- dimethyl-isoxazol-4-yl)-6-(4- morpholin-4-yl-phenyl)- pyridin-2-yloxymethyl]- thiophene-2-carboxylic acid methyl ester | Y | | 16.9 (M3) |
| 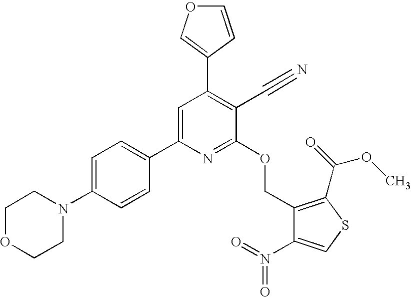 | 546.56 | 3-[3-Cyano-4-furan-3-yl-6-(4- morpholin-4-yl-phenyl)- pyridin-2-yloxymethyl]-4- nitro-thiophene-2-carboxylic acid methyl ester | Y | | |
| 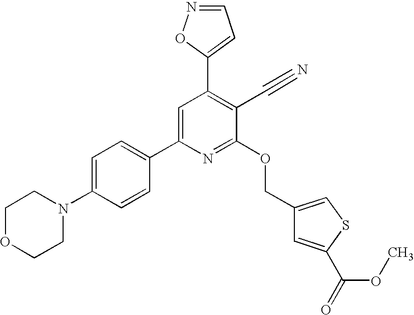 | 502.55 | 4-[3-Cyano-4-isoxazol-5-yl-6- (4-morpholin-4-yl-phenyl)- pyridin-2-yloxymethyl]- thiophene-2-carboxylic acid methyl ester | Y | 525 (M + Na+) | 15.81 (M3) |
| 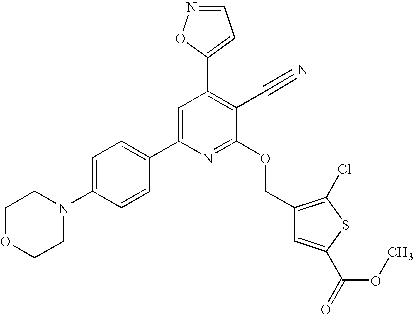 | 537 | 5-Chloro-4-[3-cyano-4- isoxazol-5-yl-6-(4-morpholin- 4-yl-phenyl)-pyridin-2- yloxymethyl]-thiophene-2- carboxylic acid methyl ester | Y | 575 (M + K+) | 17.3 (M3) |
| 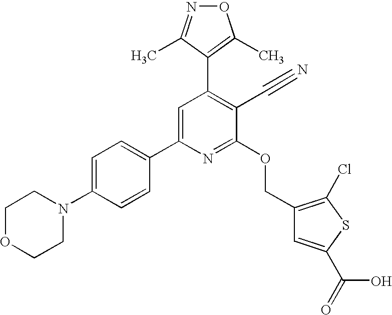 | 551.03 | 5-Chloro-4-[3-cyano-4-(3,5- dimethyl-isoxazol-4-yl)-6-(4- morpholin-4-yl-phenyl)- pyridin-2-yloxymethyl]- thiophene-2-carboxylic acid | Y | 550 (M − H+) | 15.0 (M3) |
| 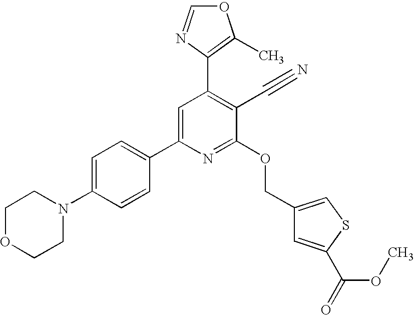 | 516.58 | 4-[3-Cyano-4-(5-methyl- oxazol-4-yl)-6-(4-morpholin- 4-yl-phenyl)-pyridin-2- yloxymethyl]-thiophene-2- carboxylic acid methyl ester | Y | 539 (M + Na+) | |
| 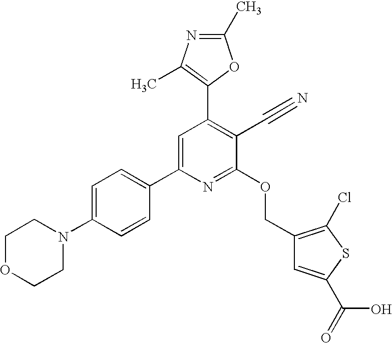 | 551.03 | 5-Chloro-4-[3-cyano-4-(2,4- dimethyl-oxazol-5-yl)-6-(4- morpholin-4-yl-phenyl)- pyridin-2-yloxymethyl]- thiophene-2-carboxylic acid | Y | 550 (M − H+) | 14.91 (M3) |
| 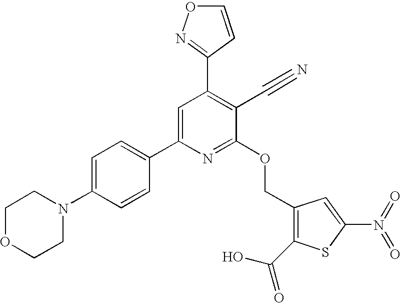 | 533.52 | 2-[3-Cyano-4-isoxazol-3-yl-6- (4-morpholin-4-yl-phenyl)- pyridin-2-yloxymethyl]-4- nitro-cyclopenta-1,3- dienecarboxylic acid | Y | 532 (M − H+) | 13.46 (M3) |
| 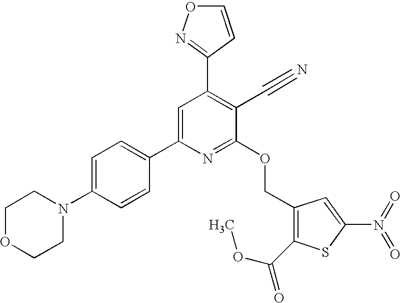 | 547.55 | 2-[3-Cyano-4-isoxazol-3-yl-6- (4-morpholin-4-yl-phenyl)- pyridin-2-yloxymethyl]-4- nitro-cyclopenta-1,3- dienecarboxylic acid methyl ester | 570 (M + Na+) | 15.07 (M3) | |
| 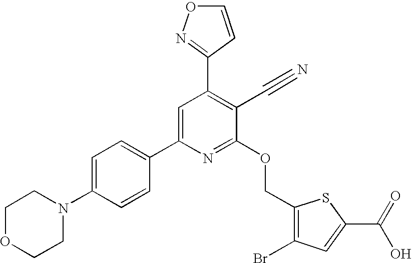 | 567.42 | 3-Bromo-4-[3-cyano-4- isoxazol-3-yl-6-(4-morpholin- 4-yl-phenyl)-pyridin-2- yloxymethyl]cyclopenta-1,3- dienecarboxylic acid | Y | 566 (M − H+) | 14.80 (M3) |
| 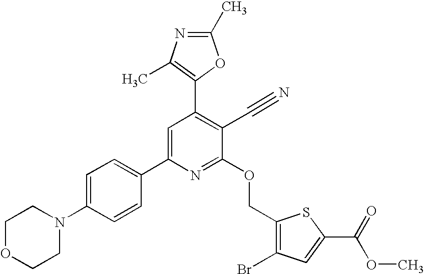 | 609.5 | 4-Bromo-5-[3-cyano-4-(2,4- dimethyl-oxazol-5-yl)-6-(4- morpholin-4-yl-phenyl)- pyridin-2-yloxymethyl]- thiophene-2-carboxylic acid methyl ester | Y | 633 (M + Na+) | 16.85 (M3) |
| 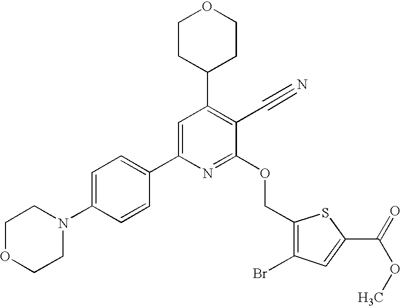 | 598.52 | 4-Bromo-5-[3-cyano-6-(4- morpholin-4-yl-phenyl)-4- (tetrahydro-pyran-4-yl)- pyridin-2-yloxymethyl]- thiophene-2-carboxylic acid | Y | 598/599 (M + H+) | 16.9 (M3) |
| 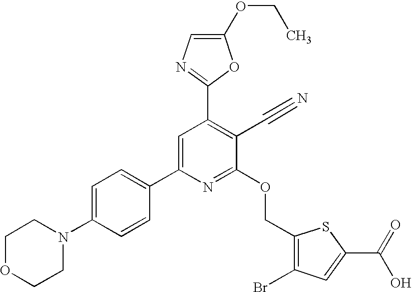 | 611.48 | 4-Bromo-5-[3-cyano-4-(5- ethoxy-oxazol-2-yl)-6-(4- morpholin-4-yl-phenyl)- pyridin-2-yloxymethyl]- thiophene-2-carboxylic acid | Y | 611/612 (M + H+) | 15.7 (M3) |
| 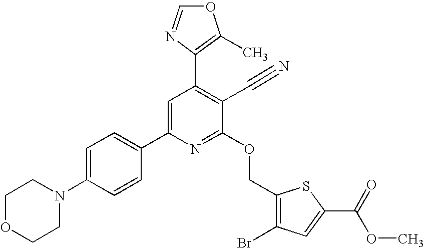 | 595.48 | 4-Bromo-5-[3-cyano-4-(5- methyl-oxazol-4-yl)-6-(4- morpholin-4-yl-phenyl)- pyridin-2-yloxymethyl]- thiophene-2-carboxylic acid methyl ester | Y | 619 (M + Na+) | 16.32 (M3) |
| 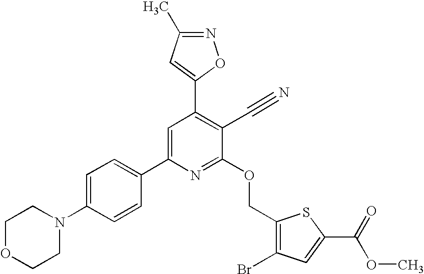 | 595.48 | 4-Bromo-5-[3-cyano-4-(3- methyl-isoxazol-5-yl)-6-(4- morpholin-4-yl-phenyl)- pyridin-2-yloxymethyl]- thiophene-2-carboxylic acid methyl ester | Y | 597 (M + H+) | 17.3 (M3) |
| 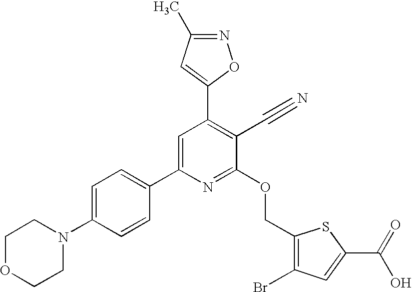 | 581.45 | 4-Bromo-5-[3-cyano-4-(3- methyl-isoxazol-5-yl)-6-(4- morpholin-4-yl-phenyl)- pyridin-2-yloxymethyl]- thiophene-2-carboxylic acid | Y | 582 (M + H+) | 15.55 (M3) |
| 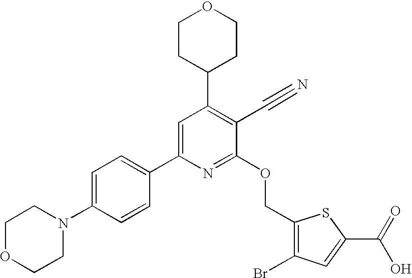 | 584.49 | 4-Bromo-5-[3-cyano-6-(4- morpholin-4-yl-phenyl)-4- (tetrahydro-pyran-4-yl)- pyridin-2-yloxymethyl]- thiophene-2-carboxylic acid | Y | 584/585 (M + H+) | 14.86 (M3) |
| 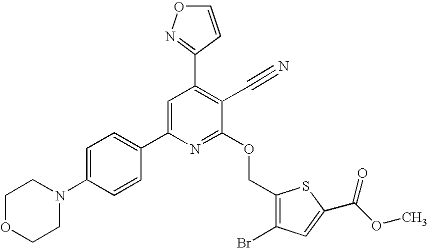 | 581.45 | 4-Bromo-5-[3-cyano-4- isoxazol-3-yl-6-(4-morpholin- 4-yl-phenyl)-pyridin-2- yloxymethyl]-thiophene-2- carboxylic acid methyl ester | Y | 605 (M + Na+) | 16.5 (M3) |
| 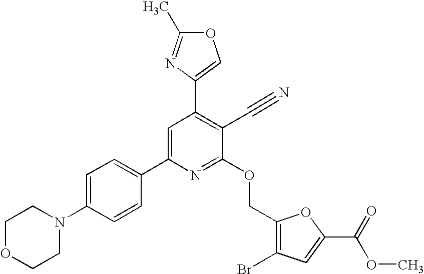 | 595.48 | 4-Bromo-5-[3-cyano-4-(2- methyl-oxazol-4-yl)-6-(4- morpholin-4-yl-phenyl)- pyridin-2-yloxymethyl]- thiophene-2-carboxylic acid methyl ester | Y | 596/597 (M + H+) | 17.5 (M3) |
| 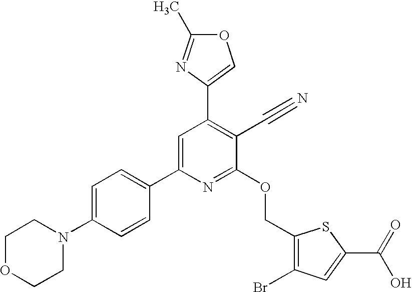 | 581.45 | 4-Bromo-5-[3-cyano-4-(2- methyl-oxazol-4-yl)-6-(4- morpholin-4-yl-phenyl)- pyridin-2-yloxymethyl]- thiophene-2-carboxylic acid | Y | 581/582 (M + H+) | |
| 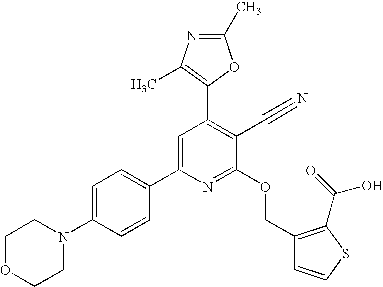 | 516.58 | 3-[3-Cyano-4-(2,4-dimethyl- oxazol-5-yl)-6-(4-morpholin- 4-yl-phenyl)-pyridin-2- yloxymethyl]-thiophene-2- carboxylic acid | Y | 517 (M + H+) | 13.6 (M3) |
| 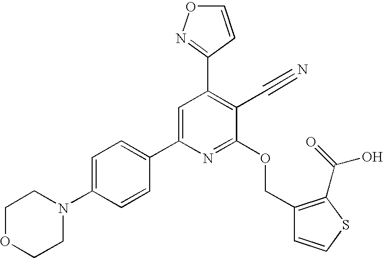 | 488.53 | 3-[3-Cyano-4-isoxazol-3-yl-6- (4-morpholin-4-yl-phenyl)- pyridin-2-yloxymethyl]- thiophene-2-carboxylic acid | Y | 489 (M + H+) | 13.8 (M3) |
| 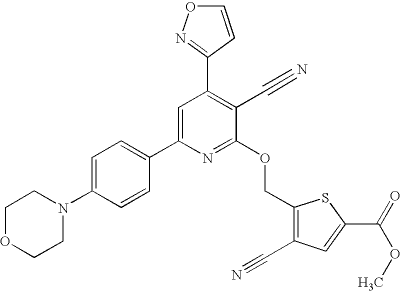 | 527.56 | 4-Cyano-5-[3-cyano-4- isoxazol-3-yl-6-(4-morpholin- 4-yl-phenyl)-pyridin-2- yloxymethyl]-thiophene-2- carboxylic acid methyl ester | Y | 550 (M + Na+) | 15.3 (M3) |
| 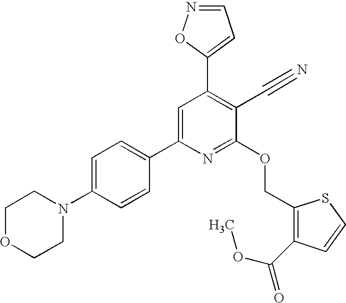 | 502.55 | 2-[3-Cyano-4-isoxazol-5-yl-6- (4-morpholin-4-yl-phenyl)- pyridin-2-yloxymethyl]- thiophene-3-carboxylic acid methyl ester | Y | 525 (M + Na+) | 16.5 (M3) |
| 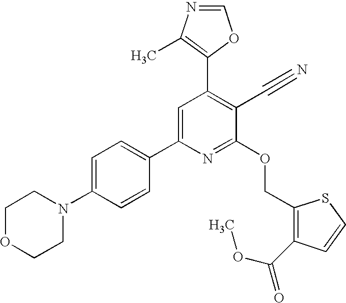 | 516.58 | 2-[3-Cyano-4-(4-methyl- oxazol-5-yl)-6-(4-morpholin- 4-yl-phenyl)-pyridin-2- yloxymethyl]-thiophene-3- carboxylic acid methyl ester | Y | 539 (M + Na+) | 15.7 (M3) |
| 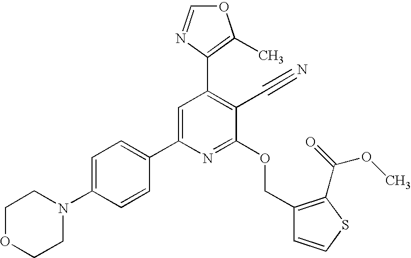 | 516.58 | 3-[3-Cyano-4-(5-methyl- oxazol-4-yl)-6-(4-morpholin- 4-yl-phenyl)-pyridin-2- yloxymethyl]-thiophene-2- carboxylic acid methyl ester | Y | 539 (M + Na+) | 15.90 (M3) |
| 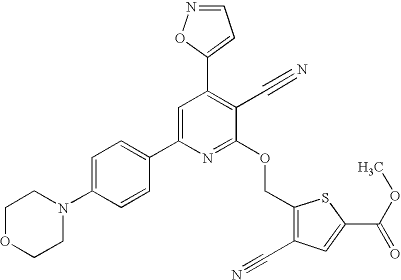 | 527.56 | 4-Cyano-5-[3-cyano-4- isoxazol-5-yl-6-(4-morpholin- 4-yl-phenyl)-pyridin-2- yloxymethyl]-thiophene-2- carboxylic acid methyl ester | Y | 550 (M + Na+) | 15.6 (M3) |
| 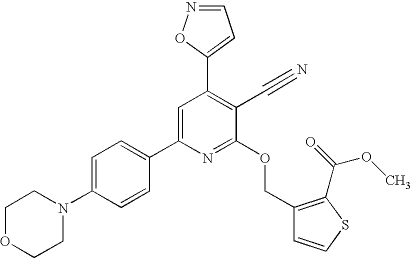 | 502.55 | 3-[3-Cyano-4-isoxazol-5-yl-6- (4-morpholin-4-yl-phenyl)- pyridin-2-yloxymethyl]- thiophene-2-carboxylic acid methyl ester | Y | 525 (M + Na+) | 16.5 (M3) |
| 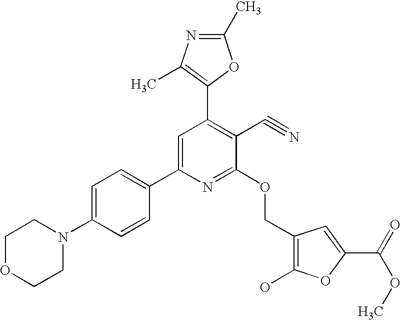 | 565.05 | 5-Chloro-4-[3-cyano-4-(2,4- dimethyl-oxazol-5-yl)-6-(4- morpholin-4-yl-phenyl)- pyridin-2-yloxymethyl]- thiophene-2-carboxylic acid methyl ester | Y | 565/566 (M + H+) | 16.9 (M3) |
| 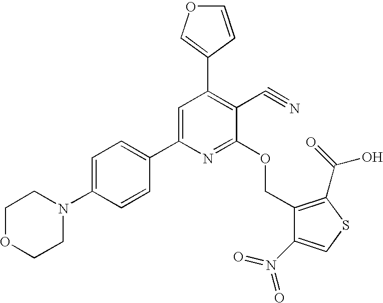 | 532.54 | 3-[3-Cyano-4-furan-3-yl-6-(4- morpholin-4-yl-phenyl)- pyridin-2-yloxymethyl]-4- nitro-thiophene-2-carboxylic acid | Y | | 15.4 (M3) |
| 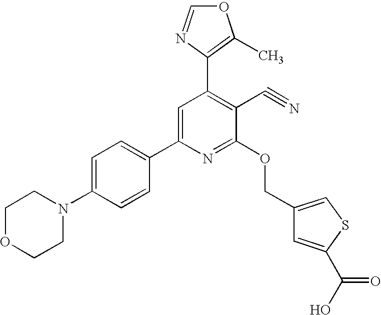 | 502.55 | 4-[3-Cyano-4-(5-methyl- oxazol-4-yl)-6-(4-morpholin- 4-yl-phenyl)-pyridin-2- yloxymethyl]-thiophene-2- carboxylic acid | Y | 501 (M − H+) | 13.38 (M3) |
| 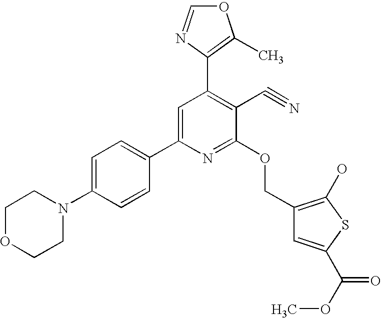 | 551.03 | 5-Chloro-4-[3-cyano-4-(5- methyl-oxazol-4-yl)-6-(4- morpholin-4-yl-phenyl)- pyridin-2-yloxymethyl]- thiophene-2-carboxylic acid methyl ester | Y | 588 (M + Na+) | 16.83 (M3) |
| 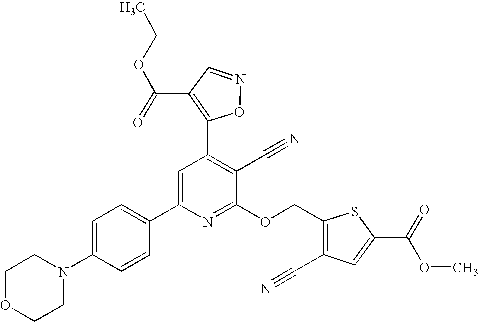 | 599.63 | 5-[3-Cyano-2-(3-cyano-5- methoxycarbonyl-thiophen-2- ylmethoxy)-6-(4-morpholin-4- yl-phenyl)-pyridin-4-yl]- isoxazole-4-carboxylic acid ethyl ester | Y | 622 (M + Na+) | 17.74 (M3) |
| 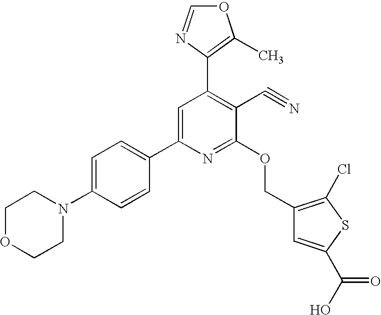 | 537 | 5-Chloro-4-[3-cyano-4-(5- methyl-oxazol-4-yl)-6-(4- morpholin-4-yl-phenyl)- pyridin-2-yloxymethyl]- thiophene-2-carboxylic acid | Y | 536 (M − H+) | 15.02 (M3) |
| 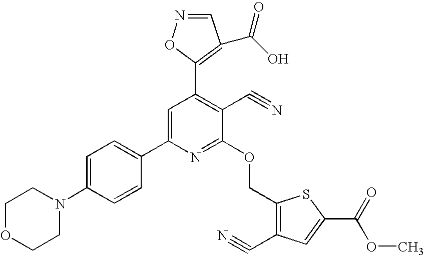 | 571.57 | 5-[3-Cyano-2-(3-cyano-5- methoxycarbonyl-thiophen-2- ylmethoxy)-6-(4-morpholin-4- yl-phenyl)-pyridin-4-yl]- isoxazole-4-carboxylic acid | Y | | |
| 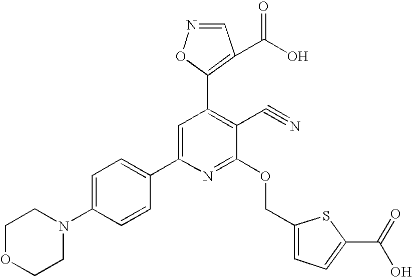 | 532.54 | 5-[2-(5-Carboxy-thiophen-2- ylmethoxy)-3-cyano-6-(4- morpholin-4-yl-phenyl)- pyridin-4-yl]-isoxazole-4- carboxylic acid | Y | | 10.66 (M3) |
| 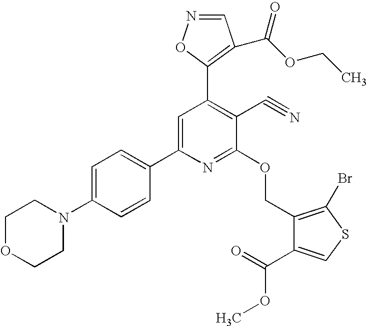 | 653.51 | 5-[2-(2-Bromo-4- methoxycarbonyl-thiophen-3- ylmethoxy)-3-cyano-6-(4- morpholin-4-yl-phenyl)- pyridin-4-yl]-isoxazole-4- carboxylic acid ethyl ester | Y | 675 (M + Na+) | 15.3 (M3) |
| 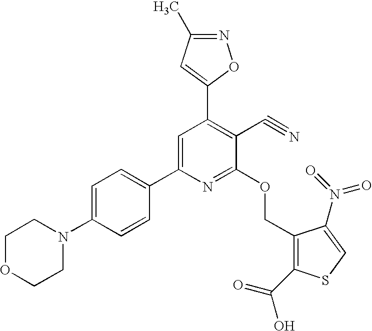 | 547.55 | 3-[3-Cyano-4-(3-methyl- isoxazol-5-yl)-6-(4-morpholin- 4-yl-phenyl)-pyridin-2- yloxymethyl]-4-nitro- thiophene-2-carboxylic acid | Y | 546 (M − H+) | 13.72 (M3) |
| 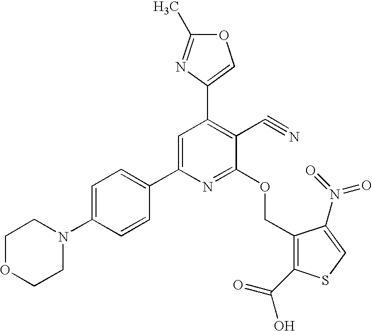 | 547.55 | 3-[3-Cyano-4-(2-methyl- oxazol-4-yl)-6-(4-morpholin- 4-yl-phenyl)-pyridin-2- yloxymethyl]-4-nitro- thiophene-2-carboxylic acid | Y | 546 (M − H+) | 13.98 (M3) |
| 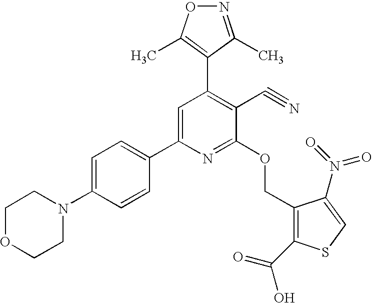 | 561.58 | 3-[3-Cyano-4-(3,5-dimethyl- isoxazol-4-yl)-6-(4-morpholin- 4-yl-phenyl)-pyridin-2- yloxymethyl]-4-nitro- thiophene-2-carboxylic acid | Y | 560 (M − H+) | 14.23 (M3) |
| 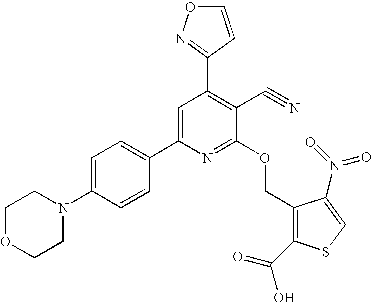 | 533.52 | 3-[3-Cyano-4-isoxazol-3-yl-6- (4-morpholin-4-yl-phenyl)- pyridin-2-yloxymethyl]-4- nitro-thiophene-2-carboxylic acid | Y | 532 (M − H+) | 14.23 (M3) |
| 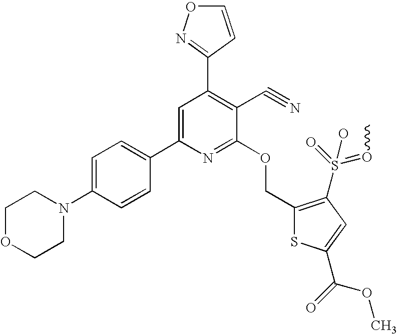 | 580.64 | 5-[3-Cyano-4-isoxazol-3-yl-6- (4-morpholin-4-yl-phenyl)- pyridin-2-yloxymethyl]-4- methanesulfonyl-thiophene- 2-carboxylic acid methyl ester | Y | 603 (M + Na+) | 14.45 (M3) |
| 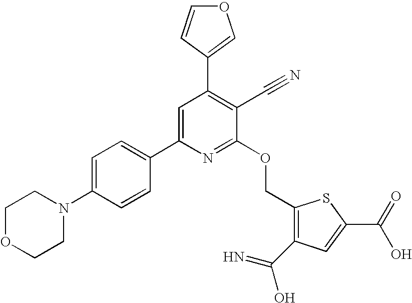 | 530.56 | 5-[3-Cyano-4-furan-3-yl-6-(4- morpholin-4-yl-phenyl)- pyridin-2-yloxymethyl]-4- hydroxycarbonimidoyl- thiophene-2-carboxylic acid | Y | 529 (M − H+) | 12.27 (M3) |
| 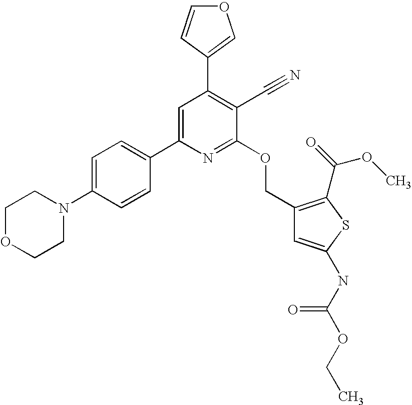 | 588.64 | 3-[3-Cyano-4-furan-3-yl-6-(4- morpholin-4-yl-phenyl)- pyridin-2-yloxymethyl]-5- ethoxycarbonylamino- thiophene-2-carboxylic acid methyl ester | Y | 611 (M + Na+) | 14.12 (M3) |
| 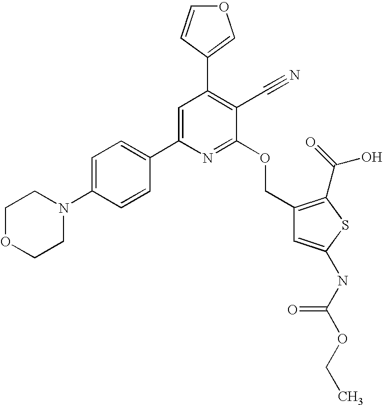 | 574.62 | 3-[3-Cyano-4-furan-3-yl-6-(4- morpholin-4-yl-phenyl)- pyridin-2-yloxymethyl]-5- ethoxycarbonylamino- thiophene-2-carboxylic acid | Y | 573 (M − H+) | 13.67 (M3) |
| 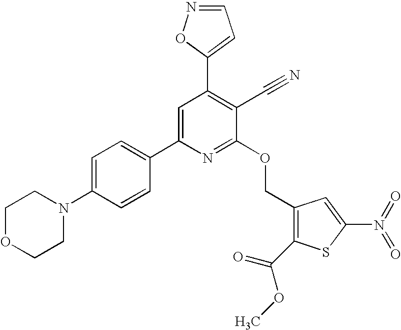 | 547.55 | 3-[3-Cyano-4-isoxazol-5-yl-6- (4-morpholin-4-yl-phenyl)- pyridin-2-yloxymethyl]-5- nitro-thiophene-2-carboxylic acid methyl ester | Y | | 13.8 (M3) |
| 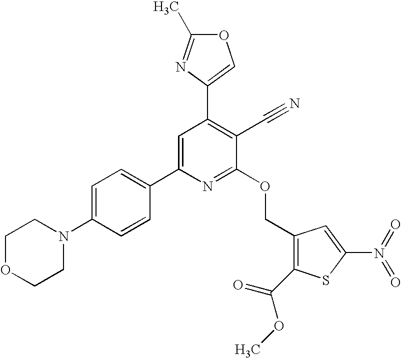 | 561.58 | 3-[3-Cyano-4-(2-methyl- oxazol-4-yl)-6-(4-morpholin- 4-yl-phenyl)-pyridin-2- yloxymethyl]5-nitro- thiophene-2-carboxylic acid methyl ester | Y | 584 (M + Na+) | 15.91 (M3) |
| 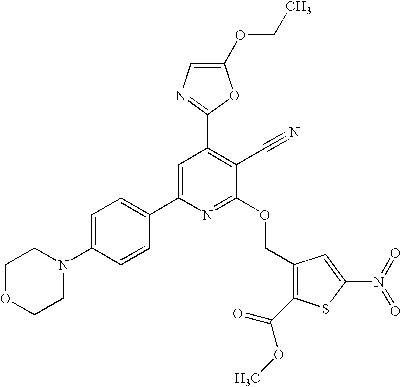 | 591.6 | 3-[3-Cyano-4-(5-ethoxy- oxazol-2-yl)-6-(4-morpholin- 4-yl-phenyl)-pyridin-2- yloxymethyl]-5-nitro- thiophene-2-carboxylic acid methyl ester | Y | 614 (M + Na+) | 14.71 (M3) |
| 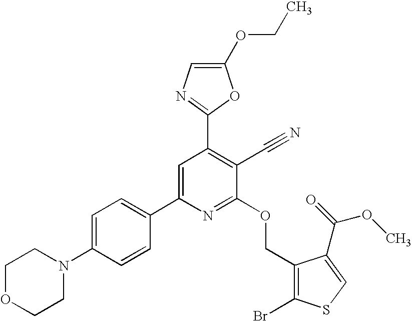 | 625.5 | 5-Bromo-4-[3-cyano-4-(5- ethoxy-oxazol-2-yl)-6-(4- morpholin-4-yl-phenyl)- pyridin-2-yloxymethyl]- thiophene-3-carboxylic acid methyl ester | y | 649 (M + Na+) | 15.07 (M3) |
| 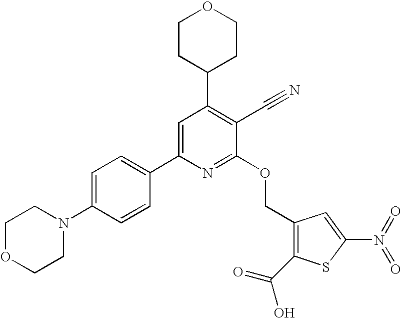 | 550.59 | 3-[3-Cyano-6-(4-morpholin-4- yl-phenyl)-4-(tetrahydro- pyran-4-yl)-pyridin-2- yloxymethyl]-5-nitro- thiophene-2-carboxylic acid | Y | 549 (M − H+) | 11.49 (M3) |
| 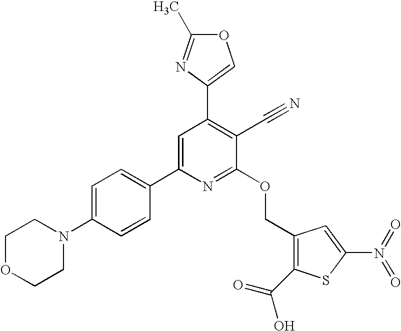 | 547.55 | 3-[3-Cyano-4-(2-methyl- oxazol-4-yl)-6-(4-morpholin- 4-yl-phenyl)-pyridin-2- yloxymethyl]-5-nitro- thiophene-2-carboxylic acid | Y | 546 (M − H+) | 12.03 (M3) |
| 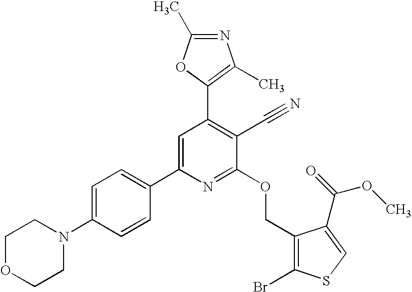 | 609.5 | 5-Bromo-4-113-cyano-4-(2,4- dimethyl-oxazol-5-yl)-6-(4- morpholin-4-yl-phenyl)- pyridin-2-yloxymethyl]- thiophene-3-carboxylic acid methyl ester | Y | 609.0/610 (M + H+) | 14.84 (M3) |
| 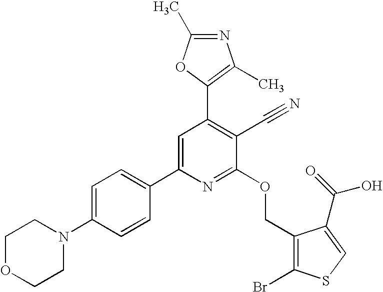 | 595.48 | 5-Bromo-4-[3-cyano-4-(2,4- dimethyl-oxazol-5-yl)-6-(4- morpholin-4-yl-phenyl)- pyridin-2-yloxymethyl]- thiophene-3-carboxylic acid | Y | 593 (M − H+) | 14.27 (M3) |
| 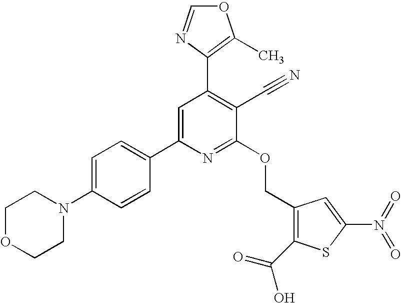 | 547.55 | 3-[3-Cyano-4-(5-methyl- oxazol-4-yl)-6-(4-morpholin- 4-yl-phenyl)-pyridin-2- yloxymethyl]-5-nitro- thiophene-2-carboxylic acid | Y | | 10.95 (M3) |
| 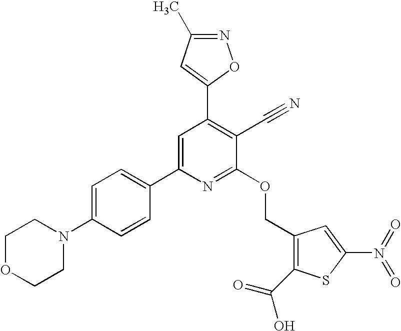 | 547.55 | 3-[3-Cyano-4-(3-methyl- isoxazol-5-yl)-6-(4-morpholin- 4-yl-phenyl)-pyridin-2- yloxymethyl]-5-nitro- thiophene-2-carboxylic acid | Y | 546 (M − H+) | 14.68 (M3) |
| 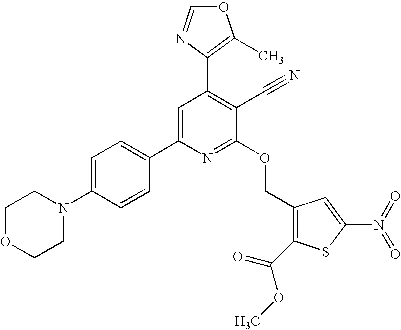 | 561.58 | 3-[3-Cyano-4-(5-methyl- oxazol-4-yl)-6-(4-morpholin- 4-yl-phenyl)-pyridin-2- yloxymethyl]-5-nitro- thiophene-2-carboxylic acid methyl ester | Y | 584 (M + Na+) | 15.2 (M3) |
| 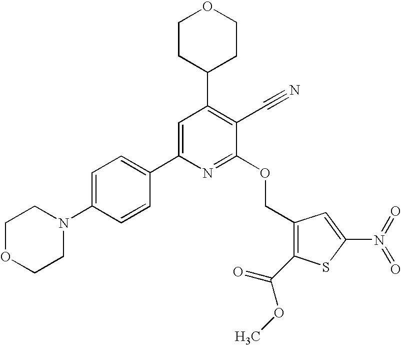 | 564.62 | 3-[3-Cyano-6-(4-morpholin-4- yl-phenyl)-4-(tetrahydro- pyran-4-yl)-pyridin-2- yloxymethyl]5-nitro- thiophene-2-carboxylic acid methyl ester | Y | 587 (M + Na+) | 13.0 (M3) |
| 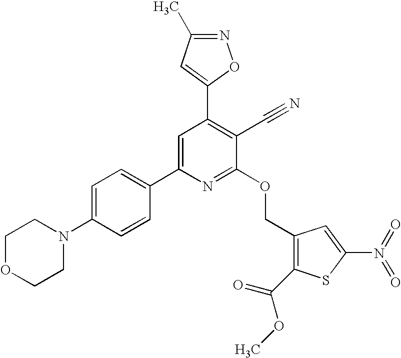 | 561.58 | 3-[3-Cyano-4-(3-methyl- isoxazol-5-yl)-6-(4-morpholin- 4-yl-phenyl)-pyridin-2- yloxymethyl]5-nitro- thiophene-2-carboxylic acid methyl ester | Y | 584 (M + Na+) | 16.2 (M3) |
| 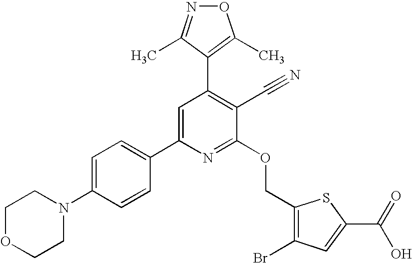 | 595.48 | 4-Bromo-5-[3-cyano-4-(3,5- dimethyl-isoxazol-4-yl)-6-(4- morpholin-4-yl-phenyl)- pyridin-2-yloxymethyl]- thiophene-2-carboxylic acid | Y | 593 (M − H+) | 14.95 (M3) |
| 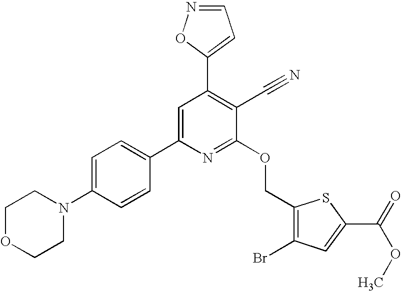 | 581.45 | 4-Bromo-5-[3-cyano-4- isoxazol-5-yl-6-(4-morpholin- 4-yl-phenyl)-pyridin-2- yloxymethyl]-thiophene-2- carboxylic acid methyl ester | Y | 581/582 (M + H+) | 16.83 (M3) |
| 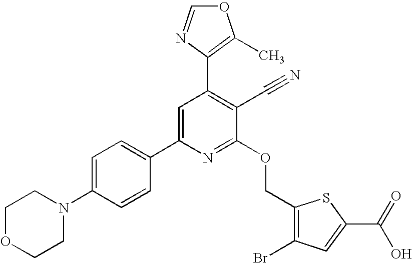 | 581.45 | 4-Bromo-5-[3-cyano-4-(5- methyl-oxazol-4-yl)-6-(4- morpholin-4-yl-phenyl)- pyridin-2-yloxymethyl]- thiophene-2-carboxylic acid | Y | 583 (M + H+) | 14.55 (M3) |
| 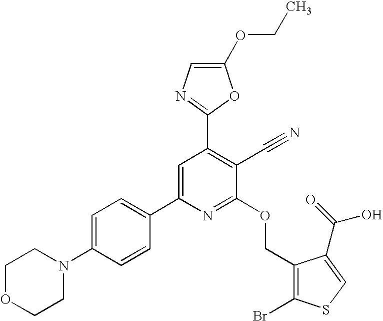 | 611.48 | 5-Bromo-4-[3-cyano-4-(5- ethoxy-oxazol-2-yl)-6-(4- morpholin-4-yl-phenyl)- pyridin-2-yloxymethyl]- thiophene-3-carboxylic acid | Y | 609 (M − H+) | 14.8 (M3) |
| 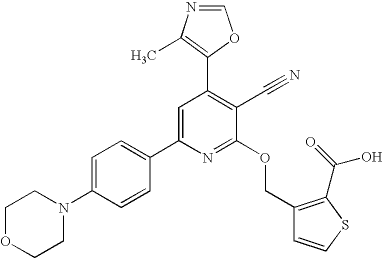 | 502.55 | 3-[3-Cyano-4-(4-methyl- oxazol-5-yl)-6-(4-morpholin- 4-yl-phenyl)-pyridin-2- yloxymethyl]-thiophene-2- carboxylic acid | Y | 503 (M + H+) | 12.3 (M3) |
| 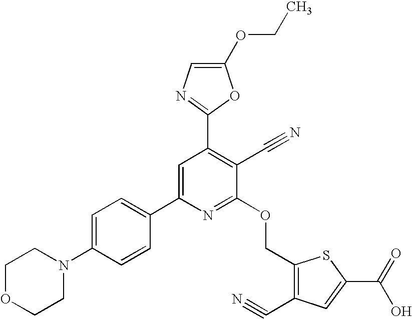 | 557.59 | 4-Cyano-5-[3-cyano-4-(5- ethoxy-oxazol-2-yl)-6-(4- morpholin-4-yl-phenyl)- pyridin-2-yloxymethyl]- thiophene-2-carboxylic acid | Y | 556 (M − H+) | 13.1 (M3) |
| 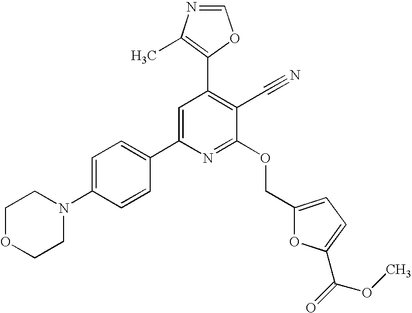 | 516.58 | 5-[3-Cyano-4-(4-methyl- oxazol-5-yl)-6-(4-morpholin- 4-yl-phenyl)-pyridin-2- yloxymethyl]-thiophene-2- carboxylic acid methyl ester | Y | 539 (M + Na+) | 15.5 (M3) |
| 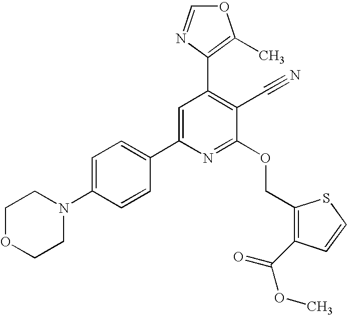 | 516.58 | 2-[3-Cyano-4-(5-methyl- oxazol-4-yl)-6-(4-morpholin- 4-yl-phenyl)-pyridin-2- yloxymethyl]-thiophene-3- carboxylic acid methyl ester | Y | 539 (M + Na+) | 16.3 (M3) |
| 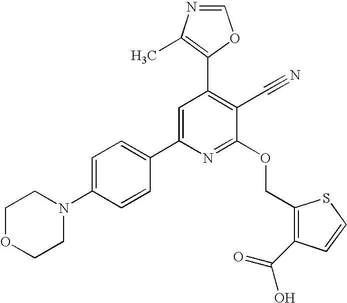 | 502.55 | 2-[3-Cyano-4-(4-methyl- oxazol-5-yl)-6-(4-morpholin- 4-yl-phenyl)-pyrindin-2- yloxymethyl]-thiophene-3- carboxylic acid | Y | 525 (M + Na+) | 11.6 (M3) |
| 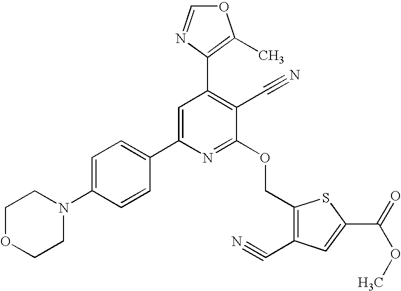 | 541.59 | 4-Cyano-5-[3-cyano-4-(5- methyl-oxazol-4-yl)-6-(4- morpholin-4-yl-phenyl)- pyridin-2-yloxymethyl]- thiophene-2-carboxylic acid methyl ester | Y | 564 (M + Na+) | 15.3 (M3) |
| 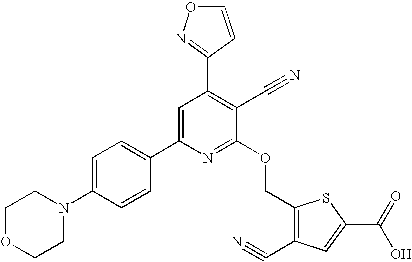 | 513.54 | 4-Cyano-5-[3-cyano-4- isoxazol-3-yl-6-(4-morpholin- 4-yl-phenyl)-pyridin-2- yloxymethyl]thiophene-2- carboxylic acid | Y | | 13.5 (M3) |
| 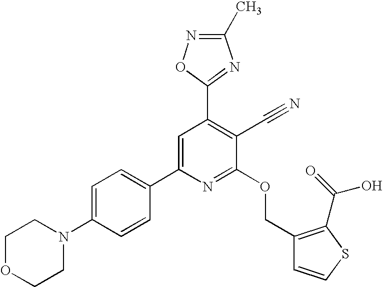 | 503.54 | 3-[3-Cyano-4-(3-methyl- [1,2,4]oxadiazol-5-yl)-6-(4- morpholin-4-yl-phenyl)- pyridin-2-yloxymethyl]- thiophene-2-carboxylic acid | Y | 540.0 (M + K+) | 14.2 (M3) |
| 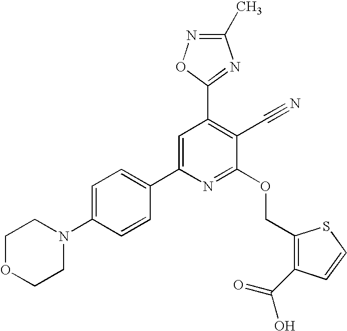 | 488.53 | 2-[3-Cyano-4-isoxazol-3-yl-6- (4-morpholin-4-yl-phenyl)- pyridin-2-yloxymethyl]- thiophene-3-carboxylic acid | Y | 489 (M + H+) | 13.9 (M3) |
| 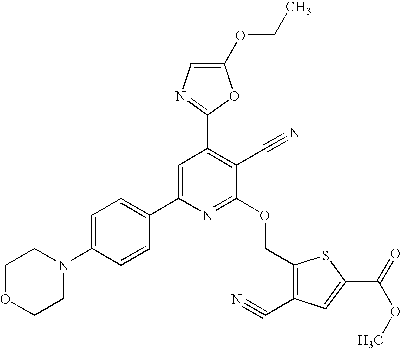 | 571.62 | 4-Cyano-5-[3-cyano-4-(5- ethoxy-oxazol-2-yl)-6-(4- morpholin-4-yl-phenyl)- pyridin-2-yloxymethyl]- thiophene-2-carboxylic acid methyl ester | Y | 594 (M + Na+) | 16.3 (M3) |
| 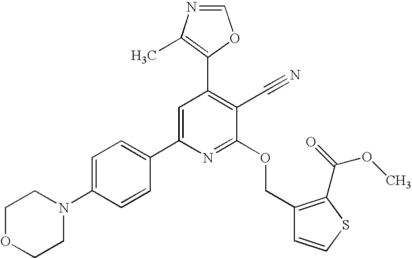 | 516.58 | 3-[3-Cyano-4-(4-methyl- oxazol-5-yl)-6-(4-morpholin- 4-yl-phenyl)-pyridin-2- yloxymethyl]-thiophene-2- carboxylic acid methyl ester | Y | | |
| 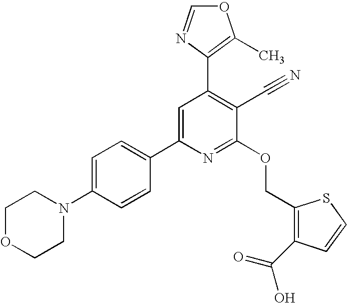 | 502.55 | 2-[3-Cyano-4-(5-methyl- oxazol-4-yl)-6-(4-morpholin- 4-yl-phenyl)-pyridin-2- yloxymethyl]thiophene-3- carboxylic acid | Y | | |
| 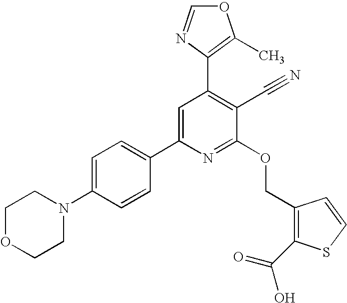 | 502.55 | 3-[3-Cyano-4-(5-methyl- oxazol-4-yl)-6-(4-morpholin- 4-yl-phenyl)-pyridin-2- yloxymethyl]-thiophene-2- carboxylic acid | Y | | |
| 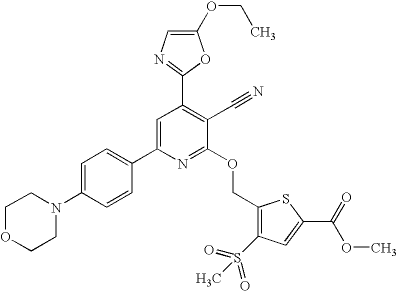 | 624.7 | 5-[3-Cyano-4-(5-ethoxy- oxazol-2-yl)-6-(4-morpholin- 4-yl-phenyl)-pyridin-2- yloxymethyl]-4- methanesulfonyl-thiophene- 2-carboxylic acid methyl ester | Y | | |
| 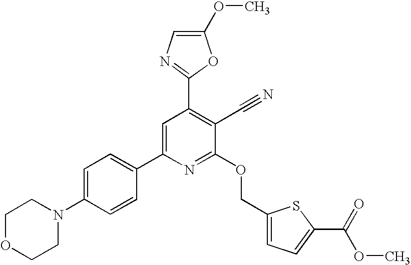 | 532.58 | 5-[3-Cyano-4-(5-methoxy- oxazol-2-yl)-6-(4-morpholin- 4-yl-phenyl)-pyridin-2- yloxymethyl]-thiophene-2- carboxylic acid methyl ester | Y | | |
| 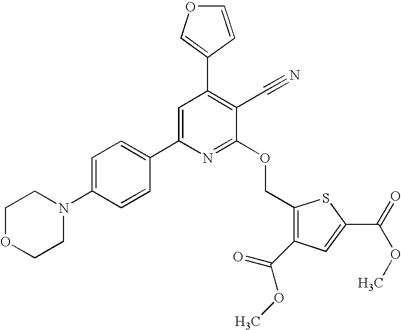 | 559.6 | 5-[3-Cyano-4-furan-3-yl-6-(4- morpholin-4-yl-phenyl)- pyridin-2-yloxymethyl]- thiophene-2,4-dicarboxylic acid dimethyl ester | Y | | |
| 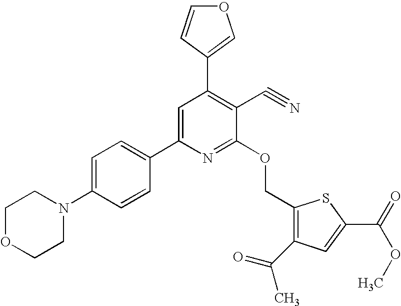 | 543.6 | 4-Acetyl-5-[3-cyano-4-furan- 3-yl-6-(4-morpholin-4-yl- phenyl)-pyridin-2- yloxymethyl]-thiophene-2- carboxylic acid methyl ester | Y | | |
| 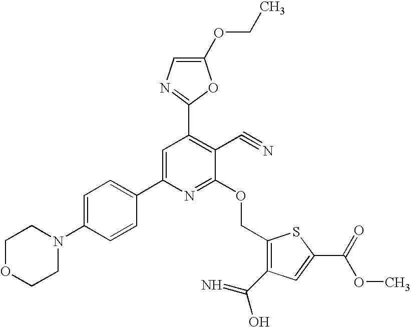 | 589.63 | 5-[3-Cyano-4-(5-ethoxy- oxazol-2-yl)-6-(4-morpholin- 4-yl-phenyl)-pyridin-2- yloxymethyl]-4- hydroxycarbonimidoyl- thiophene-2-carboxylic acid methyl ester | Y | | |
| 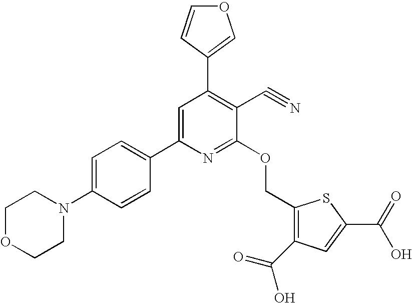 | 531.55 | 5-[3-Cyano-4-furan-3-yl-6-(4- morpholin-4-yl-phenyl)- pyridin-2-yloxymethyl]- thiophene-2,4-dicarboxylic acid | Y | | |
| 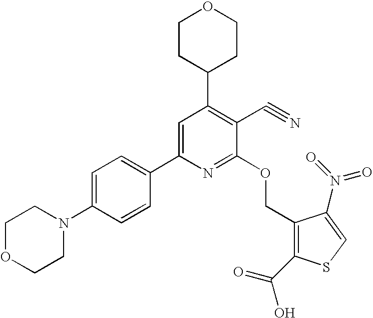 | 550.59 | 3-[3-Cyano-6-(4-morpholin-4- yl-phenyl)-4-(tetrahydro- pyran-4-yl)-pyridin-2- yloxymethyl]4-nitro- thiophene-2-carboxylic acid | Y | | |
| 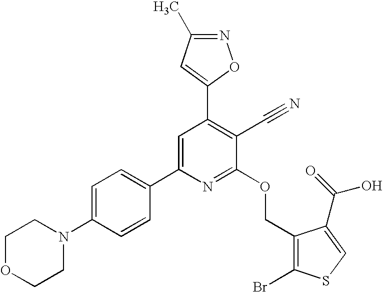 | 581.45 | 5-Bromo-4-[3-cyano-4-(3- methyl-isoxazol-5-yl)-6-(4- morpholin-4-yl-phenyl)- pyridin-2-yloxymethyl]- thiophene-3-carboxylic acid | Y | | |
| 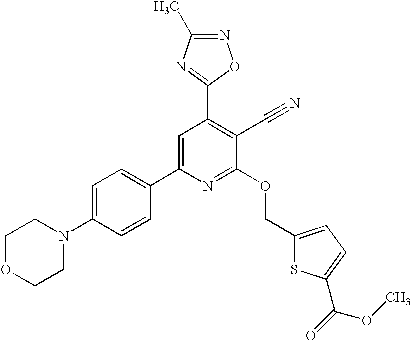 | 517.57 | 5-[3-Cyano-4-(3-methyl- [1,2,4]oxadiazol-5-yl)-6-(4- morpholin-4-yl-phenyl)- pyridin-2-yloxymethyl]- thiophene-2-carboxylic acid methyl ester | Y | | |
| 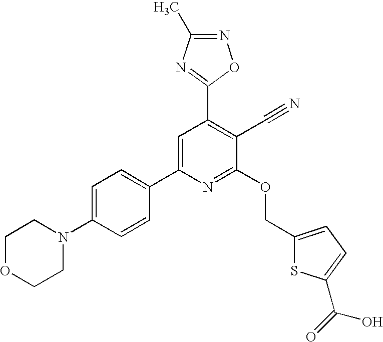 | 503.54 | 5-[3-Cyano-4-(3-methyl- [1,2,4]oxadiazol-5-yl)-6-(4- morpholin-4-yl-phenyl)- pyridin-2-yloxymethyl]- thiophene-2-carboxylic acid | Y | | |
| 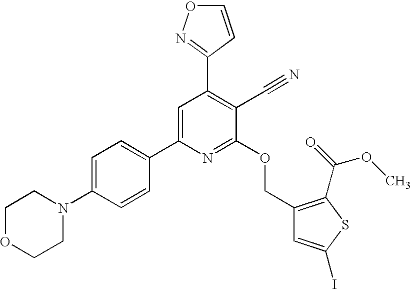 | 628.45 | 3-[3-Cyano-4-isoxazol-3-yl-6- (4-morpholin-4-yl-phenyl)- pyridin-2-yloxmethyl]-5- iodo-thiophene-2-carboxylic acid methyl ester | Y | | |
| 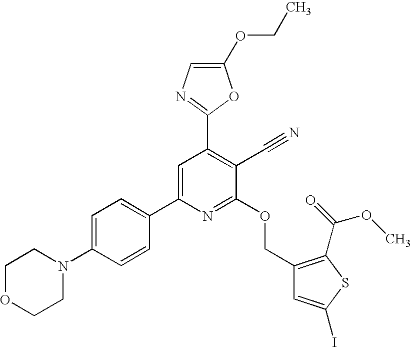 | 672.5 | 3-[3-Cyano-4-(5-ethoxy- oxazol-2-yl)-6-(4-morpholin- 4-yl-phenyl)-pyridin-2- yloxymethyl]5-iodo- thiophene-2-carboxylic acid methyl ester | Y | | |
| 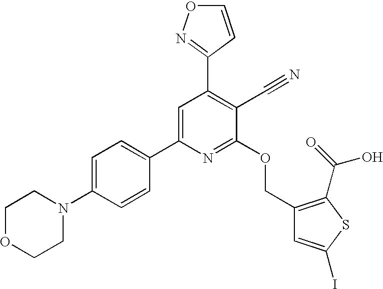 | 614.42 | 3-[3-Cyano-4-isoxazol-3-yl-6- (4-morpholin-4-yl-phenyl)- pyridin-2-yloxymethyl]-5- iodo-thiophene-2-carboxylic acid | Y | | |
| 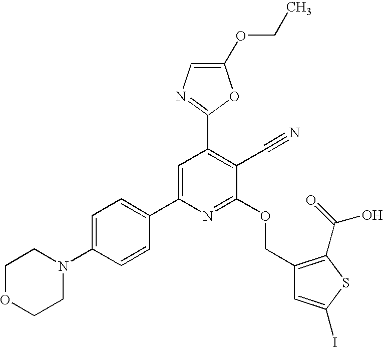 | 658.48 | 3-[3-Cyano-4-(5-ethoxy- oxazol-2-yl)-6-(4-morpholin- 4-yl-phenyl)-pyridin-2- yloxymethyl]-5-iodo- thiophene-2-carboxylic acid | Y | | |
| 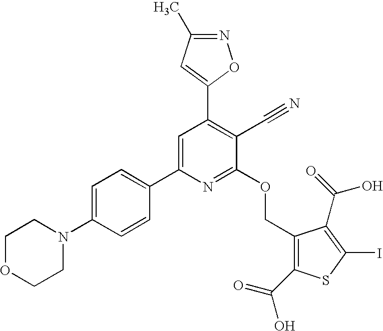 | 628.45 | 3-[3-Cyano-4-(3-methyl- isoxazol-5-yl)-6-(4-morpholin- 4-yl-phenyl)-pyridin-2- yloxymethyl]-5-iodo- thiophene-2-carboxylic acid | Y | | |
| 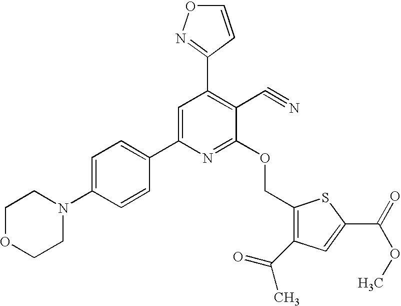 | 544.59 | 4-Acetyl-5-[3-cyano-4- isoxazol-3-yl-6-(4-morpholin- 4-yl-phenyl)-pyridin-2- yloxymethyl]-thiophene-2- carboxylic acid methyl ester | Y | | |
| 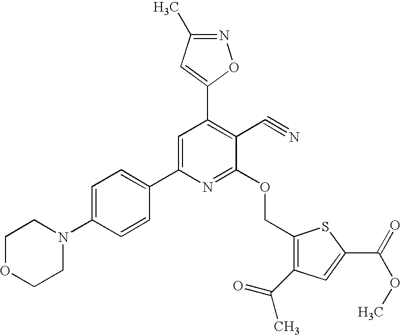 | 558.62 | 4-Acetyl-5-[3-cyano-4-(3- methyl-isoxazol-5-yl)-6-(4- morpholin-4-yl-phenyl)- pyridin-2-yloxymethyl]- thiophene-2-carboxylic acid methyl ester | Y | | |
| 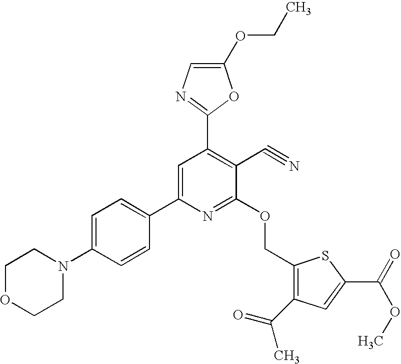 | 588.64 | 4-Acetyl-5-[3-cyano-4-(5- ethoxy-oxazol-2-yl)-6-(4- morpholin-4-yl-phenyl)- pyridin-2-yloxymethyl]- thiophene-2-carboxylic acid methyl ester | Y | | |
| 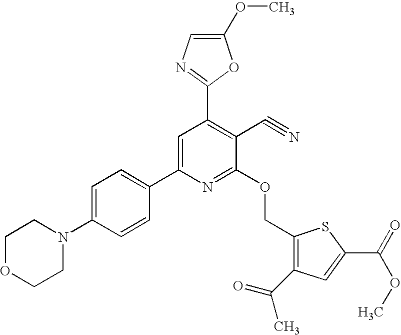 | 574.62 | 4-Acetyl-5-[3-cyano-4-(5- methoxy-oxazol-2-yl)-6-(4- morpholin-4-yl-phenyl)- pyridin-2-yloxymethyl]- thiophene-2-carboxylic acid methyl ester | Y | | |
| 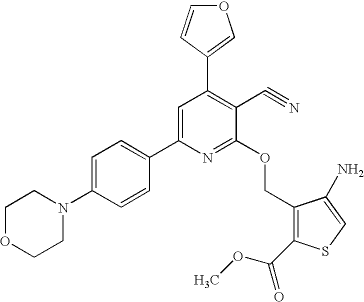 | 516.58 | 4-Amino-3-[3-cyano-4-furan- 3-yl-6-(4-morpholin-4-yl- phenyl)-pyridin-2- yloxymethyl]-thiophene-2- carboxylic acid methyl ester | Y | | |
| 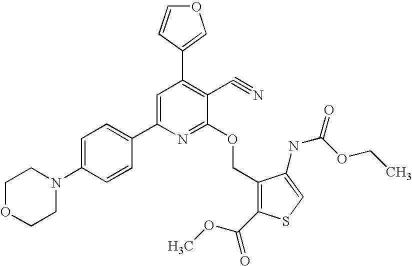 | 588.64 | 3-[3-Cyano-4-furan-3-yl-6-(4- morpholin-4-yl-phenyl)- pyridin-2-yloxymethyl]-4- ethoxycarbonylamino- thiophene-2-carboxylic acid methyl ester | Y | | |
| 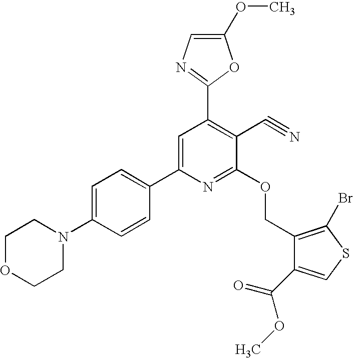 | 611.48 | 5-Bromo-4-[3-cyano-4-(5- methoxy-oxazol-2-yl)-6-(4- morpholin-4-yl-phenyl)- pyridin-2-yloxymethyl]- thiophene-3-carboxylic acid methyl ester | Y | | |
| 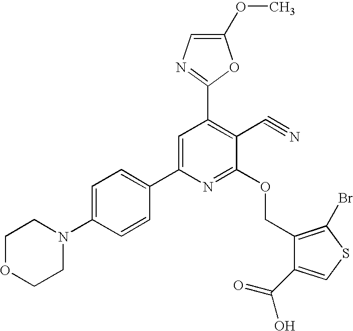 | 597.45 | 5-Bromo-4-[3-cyano-4-(5- methoxy-oxazol-2-yl)-6-(4- morpholin-4-yl-phenyl)- pyridin-2-yloxymethyl]- thiophene-3-carboxylic acid | Y | | |
| | | | Y | | |

*M3 means HPLC method 2.

### Example 19

### Biological Testing

Compounds of the present invention were tested for biological activity using the assay techniques below:

### 3′Processing/strand Transfer Combined Assay:

A combined 3′-processing/strand transfer assay procedure similar to that published (Ovenden et al. Phytochemistry. 2004 December; 65(24):3255-9.) was used. This assay was adapted to a 96 well plate format. Briefly, 400 ng of the compound to be tested is incubated with 30 nM substrate DNA, consisting of annealed U5 LTR sequence oligonucleotides tagged with Digoxigenin (DIG; 5′-ACTGCTAGAGATTTTCCACACTGACTAAAAGGGTC-DIG-3′) or biotin (5′-Bio-GACCCTTTTAGTCAGTGTGGAAAATCTCTAGCAGT-3′) so that each substrate has either a DIG or Bio tag on opposite strands. Reactions are carried out for 2 hrs at 37° C., products generated as a result of 3′processing and strand transfer activity are bound to streptavidin plates and detected with using anti-DIG-alkaline phosphatase conjugate and p-nitro phenyl phosphate substrate.

### Strand Transfer Specific Assay:

The strand transfer specific assay is of similar format to that of the 3′processing/strand transfer combined assay except that it uses a biotinylated substrate that represents a pre-processed LTR end (5′-Bio-GACCCTTTTAGTCAGTGTGGAAAATCTCTAGCA-3′).

### Inhibition of HIV Replication:

Cells are seeded into 96 well microtitre plates at 50,000 cells per 50 ul per well in RF-10 containing 2 μg/mL polybrene (RF-10/2). Compounds are prepared to 4× final concentration in RF-10/2, and 30 μL added to cells. Virus (40 μL in RF-10/2 containing 1600 pfu) is added to each well or 40 μL RF-10/2 for negative controls and for assaying compound cytotoxicity. After 24 hrs, an additional 90 μL of media or media containing 1× compound is added to each well. At 4 days post infection, 100 μL of media is removed from each well and replaced with 100 μl of fresh media with or without compound. Forty eight hours later supernatants are harvested and levels of extracellular p24 determined. Supernatants are diluted 1 in 10,000 and p24 levels assayed using the Vironostika p24 assay kit. EC₅₀ is calculated as the concentration required to inhibit HIV p24 production to 50% that of no drug controls.

The results of the assays for four compounds of the present invention are presented below in which:
IC₅₀ (3′-ST) represents the assay results for the 3′processing/strand transfer combined assay;
IC₅₀ (ST) represents the assay results for the strand transfer specific assay; and
EC50 represents the results for the inhibition of HIV replication.

Table 7 depicts the “scoring system” used in the assays.

TABLE 7
Assay scoring system

| | | |
|---|---|---|
| IC₅₀/EC₅₀ | + | >50 uM |
| | ++ | 10-50 uM |
| | +++ | <10 uM |

Assay results

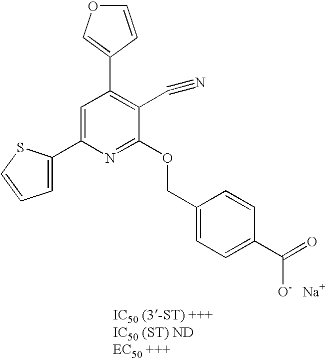

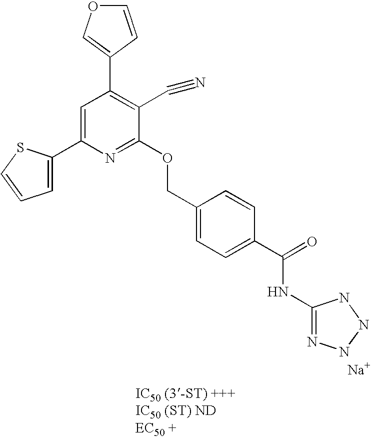

TABLE 8
Further assay results

| AvX | struct | IC50 | Ec5O |
|---|---|---|---|
| 14353 | 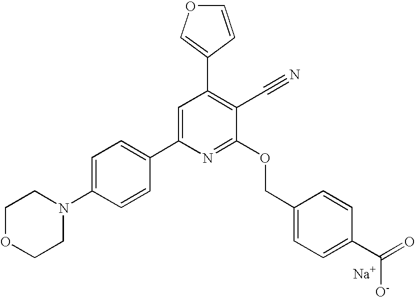 | +++ | |
| 14483 | 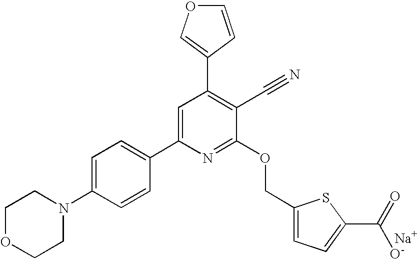 | ++ | +++ |
| 14693 | 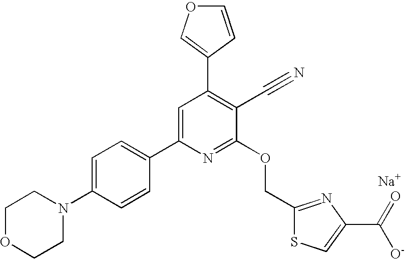 | +++ | |
| 14761 | 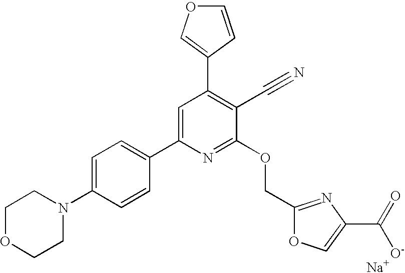 | ++ | |
| 14905 | 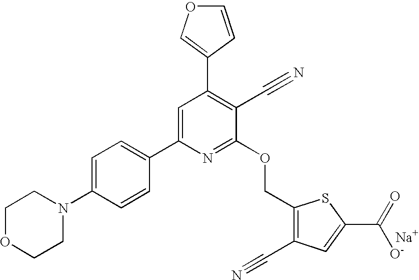 | +++ | |
| 14906 | 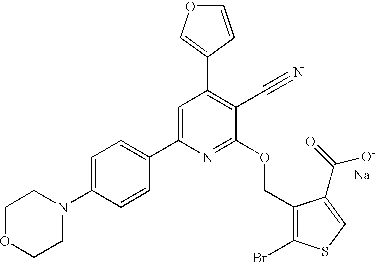 | +++ | |
| 14959 | 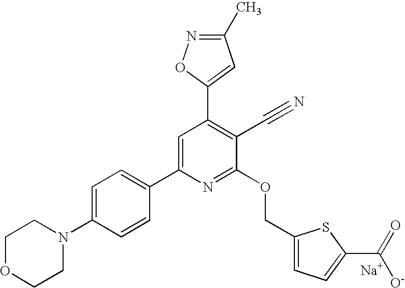 | ++ | |
| 14963 | 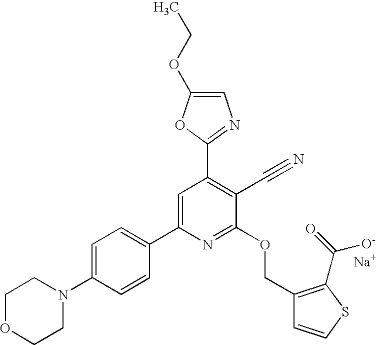 | ++ | |
| 15044 | 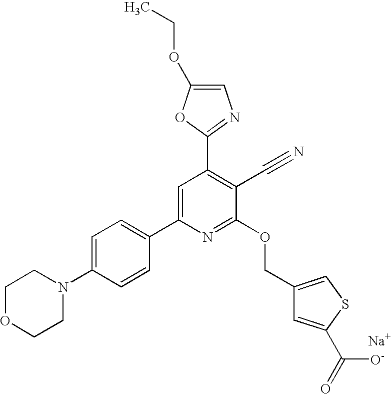 | ++ | |
| 15045 | 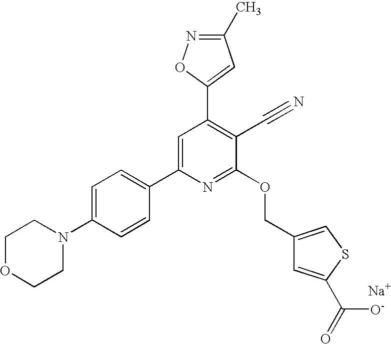 | +++ | |
| 14499 | 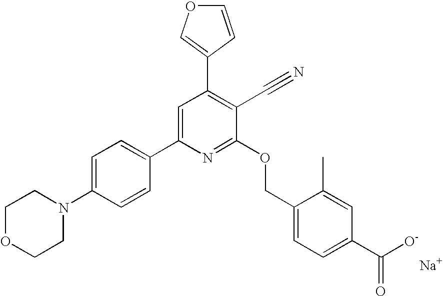 | +++ | |
| 15300 | 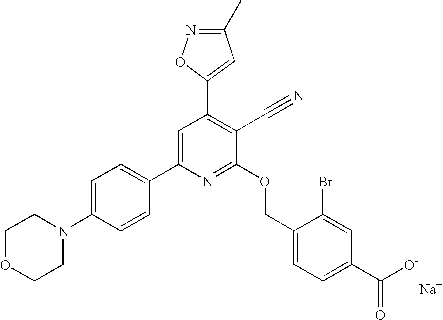 | +++ (St assay) | |
| 15303 | 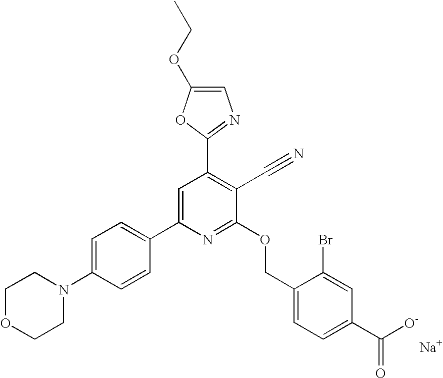 | +++ (ST assay) | |
| 15424 | 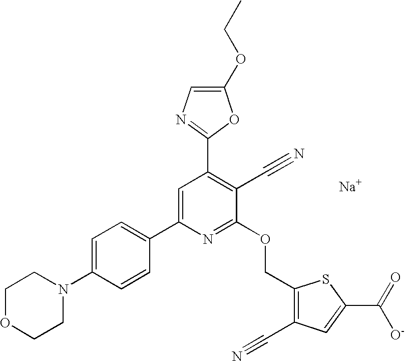 | +++ (ST) | |
| 15435 | 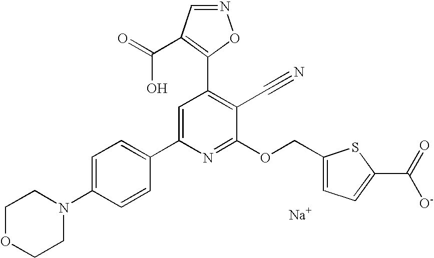 | +++ (ST) | |

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed anywhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of treatment or prophylaxis of a viral infection in a subject comprising administering to said subject an effective amount of a compound of formula I or a pharmaceutically acceptable derivative, salt or prodrug thereof wherein: 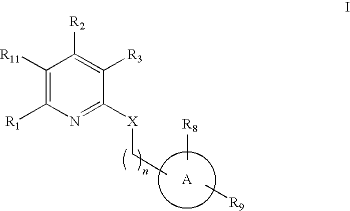
X is selected from —O—, —S—, —S(O)—, —S(O₂)— and NR₄;
R₄ is selected from H and C₁₋₃alkyl;
n is 0 or 1;
A is C₆aryl or heteroaryl;
R₁ is selected from the group consisting of hydrogen, halo, C₆₋₁₀aryl, C₆₋₁₀arylC₁₋₃alkyl, —C₁₋₁₀alkyl-O—C₁₋₁₀alkyl, heterocyclyl, hetereoaryl, C₁₋₁₀alkyl, C₁₋₁₀alkoxy, C₂₋₁₀alkenyl, C₂₋₁₀alkynyl, C₃₋₁₀cycloalkyl, —NR₅R₆, —C₆arylNR₅R₆, —C₆aryl-SO₂—NR₅R₆, —C₆aryl-heterocyclyl, —C₆aryl-SO₂-heterocyclyl; -heteroaryl-R₁₀; -Z-C₁₋₆alkylene-SO₂—R₁₂, -Z-(C₂H₄O)ₚ—R₁₂,
or R₁ and R₁₁ are joined together to form a C₃₋₄alkylene;
R₂ is selected from the group consisting of hydrogen, C₆₋₁₀aryl, C₆₋₁₀arylC₁₋₃alkyl, heterocyclyl, hetereoaryl, C₁₋₁₀alkyl, C₂₋₁₀alkenyl, C₂₋₁₀alkynyl, C₃₋₁₀cycloalkyl and —NR₅R₆, -heteroaryl-C₆₋₁₀aryl, -heteroaryl-heteroaryl;
R₃ is selected from the group consisting of hydrogen, cyano, halo, —NO₂, —C(O)NR₅R₆, —CH₂NR₅R₆, —C(O)R₇ and —CO₂R₇;
Z is absent or is selected from the group consisting of NR₅, O, S, S(O), S(O₂);
p is 1 to 3;
R₅ and R₆ are each independently selected from the group consisting of hydrogen, C₁₋₁₀alkyl, C₃₋₆cycloaklyl, C₆₋₁₀arylC₁₋₃alkyl and C₆₋₁₀aryl;
R₇ is hydrogen or C₁₋₁₀alkyl
R₁₂ is hydrogen or C₁₋₁₀alkyl;
R₈ is zero to two substituents each independently selected from the group consisting of —OH, —SO₂NH₂, —OC(O)R₇, —CO₂R₇, C₁₋₁₀alkyl, C₁₋₁₀alkoxy, halo, —NO₂, and —NR₅R₆;
R₉ is selected from the group consisting of hydrogen, cyano, —SO₂NH₂, —R₁₀, and —C(O)R₁₀;
R₁₀ is selected from OH, —C₁₋₁₀alkyl, —OC₁₋₁₀alkyl, —OC₂₋₁₀alkenyl, and —Y-heteroaryl; and
Y is absent or is selected from —O— and —NR₄—
R₁₁ is selected form the group consisting of hydrogen, C₁₋₁₀alkyl, C₁₋₁₀alkoxy; or R₁ and R₁₁ are joined together to form a C₃₋₄alkylene.

2. A method according to claim 1 wherein R₁ is selected from the group consisting of C₆₋₁₀aryl and heteroaryl.

3. A method according to claim 1 wherein R₂ is selected from the group consisting of C₆₋₁₀aryl and heteroaryl.

4. A method according to claim 1 wherein n is 1.

5. A method according to claim 1 wherein R₁₁ is hydrogen.

6. A method according to claim 1 wherein A is phenyl.

7. A method according to claim 1 wherein A is pyrdinyl.

8. A method according to claim 1 wherein A is heteroaryl selected from the group consisting of pyrrolidinyl, furanyl, and thiophene. Preferably, the heteroaryl is 2,5-substituted.

9. A method according to claim 1 wherein the compound of formula I is selected from the group consisting of:

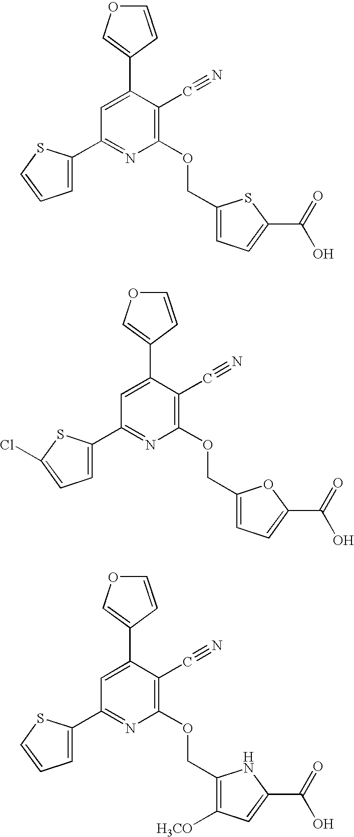

10. A method according to claim 1 wherein the compound of formula I is selected from the group consisting of:

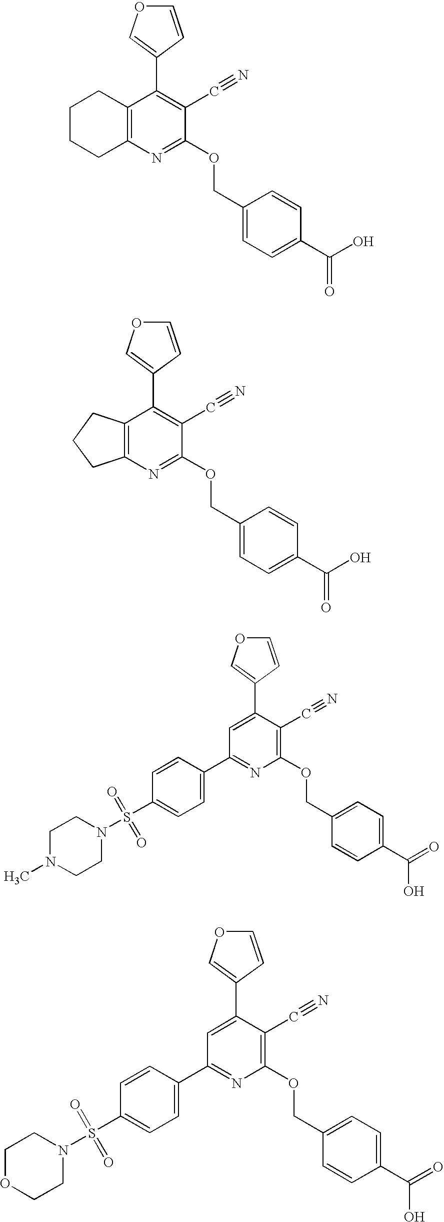

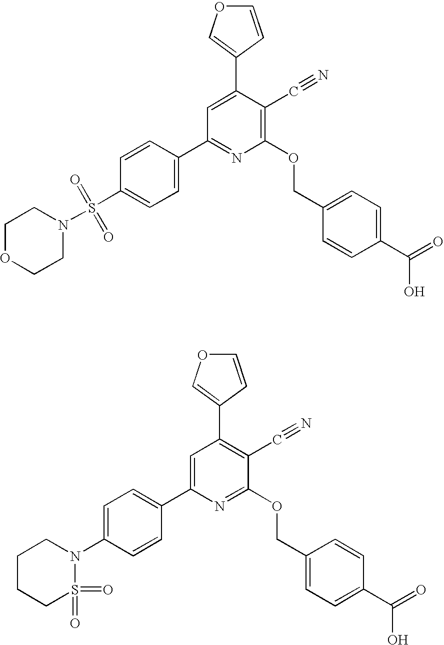

11. A compound of formula I according to claim 1 selected from the group consisting of:

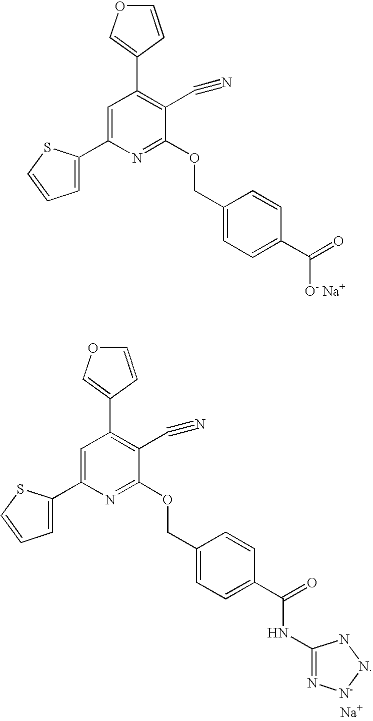

12. A compound of Formula I or a pharmaceutically acceptable derivative, salt or prodrug thereof wherein: 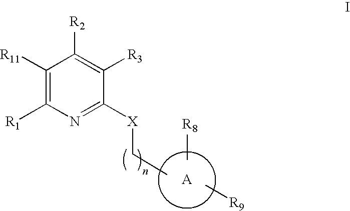
X is selected from —O—, —S—, —S(O)—, —S(O₂)— and NR₄;
R₄ is selected from H and C₁₋₃alkyl;
n is 0 or 1;
A is C₆aryl or heteroaryl;
R₁ is selected from the group consisting of hydrogen, halo, C₆₋₁₀aryl, C₆₋₁₀arylC₁₋₃alkyl, —C₁₋₁₀alkyl-O—C₁₋₁₀alkyl, heterocyclyl, hetereoaryl, C₁₋₁₀alkyl, C₁₋₁₀alkoxy, C₂₋₁₀alkenyl, C₂₋₁₀alkynyl, C₃₋₁₀cycloalkyl, —NR₅R₆, —C₆arylNR₅R₆, —C₆aryl-SO₂—NR₅R₆, —C₆aryl-heterocyclyl, —C₆aryl-SO₂-heterocyclyl; -heteroaryl-R₁₀; -Z-C₁₋₆alkylene-SO₂—R₁₂, -Z-(C₂H₄O)ₚ—R₁₂,
or R₁ and R₁₁ are joined together to form a C₃₋₄alkylene;
R₂ is selected from the group consisting of hydrogen, C₆₋₁₀aryl, C₆₋₁₀arylC₁₋₃alkyl, heterocyclyl, hetereoaryl, C₁₋₁₀alkyl, C₂₋₁₀alkenyl, C₂₋₁₀alkynyl, C₃₋₁₀cycloalkyl and —NR₅R₆, -heteroaryl-C₆₋₁₀aryl, -heteroaryl-heteroaryl;
R₃ is selected from the group consisting of hydrogen, cyano, halo, —NO₂, —C(O)NR₅R₆, —CH₂NR₅R₆, —C(O)R₇ and —CO₂R₇;
Z is absent or is selected from the group consisting of NR₅, O, S, S(O), S(O₂);
p is 1 to 3;
R₅ and R₆ are each independently selected from the group consisting of hydrogen, C₁₋₁₀alkyl, C₃₋₆cycloaklyl, C₆₋₁₀arylC₁₋₃alkyl and C₆₋₁₀aryl;
R₇ is hydrogen or C₁₋₁₀alkyl
R₁₂ is hydrogen or C₁₋₁₀alkyl;
R₈ is zero to two substituents each independently selected from the group consisting of —OH, —SO₂NH₂, —OC(O)R₇, —CO₂R₇, C₁₋₁₀alkyl, C₁₋₁₀alkoxy, halo, —NO₂, and —NR₅R₆;
R₉ is selected from the group consisting of hydrogen, cyano, —SO₂NH₂, —R₁₀, and —C(O)R₁₀;
R₁₀ is selected from OH, —C1-10alkyl, —OC₁₋₁₀alkyl, —OC₂₋₁₀alkenyl, and —Y-heteroaryl; and
Y is absent or is selected from —O— and —NR₄—
R₁₁ is selected form the group consisting of hydrogen, C₁₋₁₀alkyl, C₁₋₁₀alkoxy; or R₁ and R₁₁ are joined together to form a C₃₋₄alkylene.

13. A compound according to claim 12 wherein R₁ is selected from the group consisting of C₆₋₁₀aryl and heteroaryl.

14. A compound according to claim 13 wherein R₂ is selected from the group consisting of C₆₋₁₀aryl and heteroaryl.

15. A compound according to claim 12 wherein n is 1.

16. A compound according to claim 12 wherein R₁₁ is hydrogen.

17. A compound according to claim 12 A is phenyl.

18. A compound according to claim 12 A is pyrdinyl.

19. A compound according to claim 12 wherein A is heteroaryl selected from the group consisting of pyrrolidinyl, furanyl, and thiophene.

20. A compound according to claim 12 selected from the group consisting of:

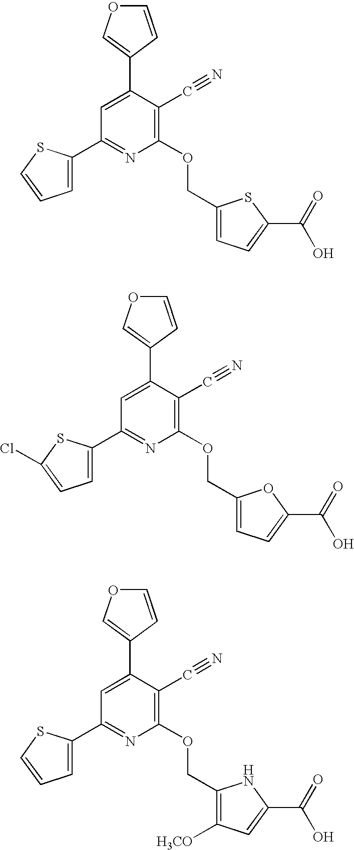

21. A compound according to claim 12 selected from the group consisting of:

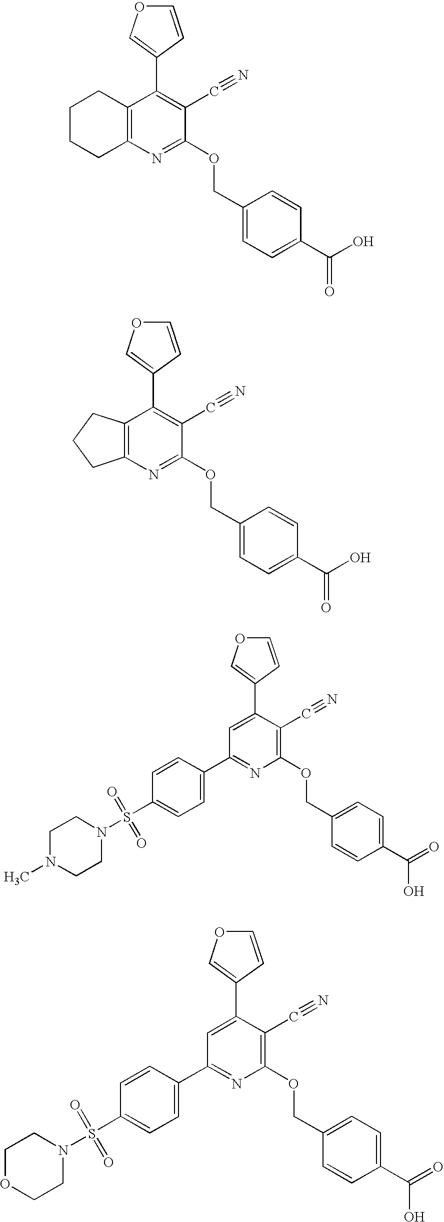

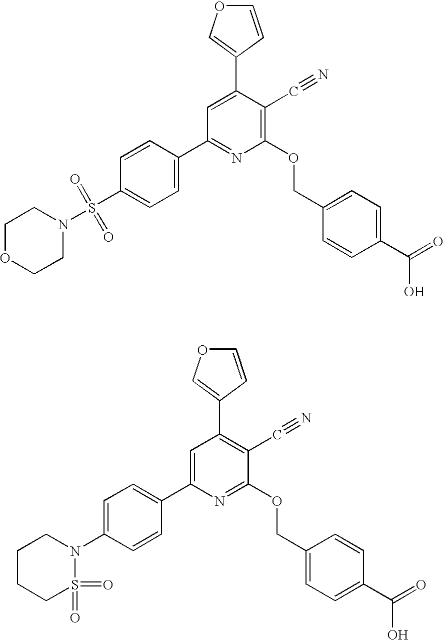

22. A compound according to claim 12 selected from the group consisting of:

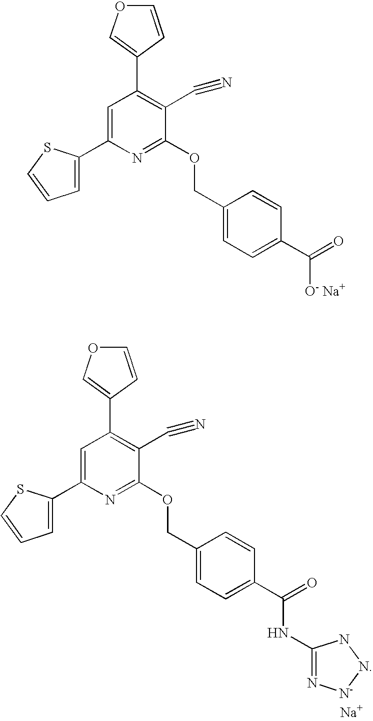

23. A pharmaceutical composition comprising a compound according to claim 12 and a pharmaceutically acceptable carrier, diluent or excipient.
